SEQUENCE LISTING (38 sequences)

SEQ ID NO: 1 - RNA, 25 nt, synthetic construct
  source 1..25: mol_type = other RNA
caccggcgaggtattcggctccgcg

SEQ ID NO: 2 - RNA, 25 nt, synthetic construct
  source 1..25: mol_type = other RNA
aaaccgcggagccgaatacctcgcc

SEQ ID NO: 3 - RNA, 25 nt, synthetic construct
  source 1..25: mol_type = other RNA
caccggcagctcttctgctcaacta

SEQ ID NO: 4 - RNA, 25 nt, synthetic construct
  source 1..25: mol_type = other RNA
aaactagttgagcagaagagctgcc

SEQ ID NO: 5 - RNA, 25 nt, synthetic construct
  source 1..25: mol_type = other RNA
caccggcgctgcgtcgtgcaccggg

SEQ ID NO: 6 - RNA, 25 nt, synthetic construct
  source 1..25: mol_type = other RNA
aaaccccggtgcacgacgcagcgcc

SEQ ID NO: 7 - RNA, 25 nt, synthetic construct
  source 1..25: mol_type = other RNA
caccgggattgggcgcctcccgaag

SEQ ID NO: 8 - RNA, 25 nt, synthetic construct
  source 1..25: mol_type = other RNA
aaaccttcgggaggcgcccaatccc

SEQ ID NO: 9 - RNA, 25 nt, synthetic construct
  source 1..25: mol_type = other RNA
caccggtcgtgcacaggtctggtaa

SEQ ID NO: 10 - RNA, 25 nt, synthetic construct
  source 1..25: mol_type = other RNA
aaacttaccagacctgtgcacgacc

SEQ ID NO: 11 - RNA, 25 nt, synthetic construct
  source 1..25: mol_type = other RNA
caccggccgtgggggtaagtaacag

SEQ ID NO: 12 - RNA, 25 nt, synthetic construct
  source 1..25: mol_type = other RNA
aaacctgttacttacccccacggcc

SEQ ID NO: 13 - RNA, 25 nt, synthetic construct
  source 1..25: mol_type = other RNA
caccgaagaccaacttctataacag

SEQ ID NO: 14 - RNA, 25 nt, synthetic construct
  source 1..25: mol_type = other RNA
aaacctgttatagaagttggtcttc

SEQ ID NO: 15 - RNA, 25 nt, synthetic construct
  source 1..25: mol_type = other RNA
caccgagtatccggttaaaagtgag

SEQ ID NO: 16 - RNA, 25 nt, synthetic construct
  source 1..25: mol_type = other RNA
aaacctcacttttaaccggatactc

SEQ ID NO: 17 - RNA, 25 nt, synthetic construct
  source 1..25: mol_type = other RNA
caccgggcaggacccacttaccccc

SEQ ID NO: 18 - RNA, 25 nt, synthetic construct
  source 1..25: mol_type = other RNA
aaacgggggtaagtgggtcctgccc

SEQ ID NO: 19 - RNA, 25 nt, synthetic construct
  source 1..25: mol_type = other RNA
caccgaggtggacacaattacatcc

SEQ ID NO: 20 - RNA, 25 nt, synthetic construct
  source 1..25: mol_type = other RNA
aaacggatgtaattgtgtccacctc

SEQ ID NO: 21 - RNA, 25 nt, synthetic construct
  source 1..25: mol_type = other RNA
caccggctcaaggagctactgacca

SEQ ID NO: 22 - RNA, 25 nt, synthetic construct
  source 1..25: mol_type = other RNA
aaactggtcagtagctccttgagcc

SEQ ID NO: 23 - RNA, 25 nt, synthetic construct
  source 1..25: mol_type = other RNA
caccgcatggcaagacccttggagg

SEQ ID NO: 24 - RNA, 25 nt, synthetic construct
  source 1..25: mol_type = other RNA
aaaccctccaagggtcttgccatgc

SEQ ID NO: 25 - RNA, 20 nt, synthetic construct
  source 1..20: mol_type = other RNA
gtcgcaggttcttggtcact

SEQ ID NO: 26 - RNA, 21 nt, synthetic construct
  source 1..21: mol_type = other RNA
catgttcacgaaagccagagc

SEQ ID NO: 27 - RNA, 20 nt, synthetic construct
  source 1..20: mol_type = other RNA
cctttcaggacgcggtgtaa

SEQ ID NO: 28 - RNA, 20 nt, synthetic construct
  source 1..20: mol_type = other RNA
aaggtactgactgcacccac

SEQ ID NO: 29 - RNA, 20 nt, synthetic construct
  source 1..20: mol_type = other RNA
tttccaacagcagcctccaa

SEQ ID NO: 30 - RNA, 21 nt, synthetic construct
  source 1..21: mol_type = other RNA
gagtgtaagctttcctgaggc

SEQ ID NO: 31 - RNA, 21 nt, synthetic construct
  source 1..21: mol_type = other RNA
ctcaaggagctactgaccagg

SEQ ID NO: 32 - RNA, 21 nt, synthetic construct
  source 1..21: mol_type = other RNA
attgtccctgttgctgtccaa

SEQ ID NO: 33 - RNA, 20 nt, synthetic construct
  source 1..20: mol_type = other RNA
ccaccctctcctcttgcatc

SEQ ID NO: 34 - RNA, 20 nt, synthetic construct
  source 1..20: mol_type = other RNA
ggaaaaaccccagctgccta

SEQ ID NO: 35 - RNA, 20 nt, synthetic construct
  source 1..20: mol_type = other RNA
tcctattaggcagctggggt

SEQ ID NO: 36 - RNA, 20 nt, synthetic construct
  source 1..20: mol_type = other RNA
cattcttctccctcccagcg

SEQ ID NO: 37 - RNA, 20 nt, synthetic construct
  source 1..20: mol_type = other RNA
ttgaggggtaggctttgcag

SEQ ID NO: 38 - RNA, 20 nt, synthetic construct
  source 1..20: mol_type = other RNA
ccctggggagatcagaggaa

(19) United States
(12) Patent Application Publication
(10) Pub. No.: US 2025/0339520 A1
(43) Pub. Date: Nov. 6, 2025

(54) COMPOSITIONS AND METHODS FOR USE IN KRAS-TARGETED THERAPIES FOR THE TREATMENT OF CANCER

(71) Applicant: RUTGERS, THE STATE UNIVERSITY OF NEW JERSEY, New Brunswick, NJ (US)

(72) Inventors: Pingping Hou, Nutley, NJ (US); Daiyong Deng, Somerset, NJ (US)

(73) Assignee: RUTGERS, THE STATE UNIVERSITY OF NEW JERSEY, New Brunswick, NJ (US)

(21) Appl. No.: 19/196,503

(22) Filed: May 1, 2025

Related U.S. Application Data
(60) Provisional application No. 63/641,226, filed on May 1, 2024.

(51) Int. Cl. A61K 39/395; A61K 31/167; A61K 31/336; A61K 31/4418; A61K 31/4439; A61K 31/444; A61K 31/4709; A61K 31/498; A61K 31/4985; A61K 31/502; A61K 31/517; A61K 31/519; A61K 31/5377; A61K 31/5386; A61K 31/553; A61K 38/13; A61K 45/06; A61P 35/00

(52) U.S. Cl. CPC A61K 39/3955; A61K 31/167; A61K 31/336; A61K 31/4418; A61K 31/4439; A61K 31/444; A61K 31/4709; A61K 31/498; A61K 31/4985; A61K 31/502; A61K 31/517; A61K 31/519; A61K 31/5377; A61K 31/5386; A61K 31/553; A61K 38/13; A61K 45/06; A61P 35/00

(57) ABSTRACT

Methods and compositions for the treatment of cancer are disclosed herein. More specifically, disclosed herein are methods and compositions for the treatment of KRASi resistant cancers using NFAT5 inhibitors.

### CROSS-REFERENCE TO RELATED APPLICATION

This application claims priority to U.S. Provisional application No. 63/641,226 filed May 1, 2024, the entire contents being incorporated herein by reference as though set forth in full.

### GOVERNMENT INTEREST STATEMENT

This invention was made with government support under K22CA251491, awarded by the National Institutes of Health. The government has certain rights in the invention.

### INCORPORATION BY REFERENCE OF MATERIAL SUBMITTED IN ELECTRONIC FORM

The Contents of the electronic sequence listing (RUT-118-US.xml; Size: 34,600 bytes; and Date of Creation: May 1, 2025) is herein incorporated by reference in its entirety.

### FIELD OF THE INVENTION

The invention relates to therapies for the treatment of cancers having resistance to KRAS-targeted therapies. More specifically, the invention relates to methods and compositions for targeting NFAT5 to prevent resistance to KRAS-targeted therapies.

### BACKGROUND OF THE INVENTION

K-ras mutant cancers are associated with genes that selectively drive the maintenance of tumors which are said to be “addicted” to or dependent on mutant K-ras. In particular pancreatic ductal adenocarcinoma (PDAC) exhibits addiction to oncogenic KRAS (Kristen Rat Sarcoma virus, KRAS*)^{6,7}, with the quasi-mesenchymal (QM) subtype demonstrating the shortest overall survival, the highest epithelial-to-mesenchymal transition (EMT) gene signature, and the least dependency on KRAS signaling across classical and exocrine-like subtypes³. Despite the significant tumor growth suppression observed with KRAS inhibitors (KRASi) in pre-clinical models^{8,9}, this anti-tumor effect is transient¹, and EMT frequently emerges as a phenotype in resistant cells^{2,10-13}. Overcoming EMT-associated therapy resistance remains a primary objective.

Transforming growth factor-beta (TGFβ), a master driver of EMT¹⁴, is abundant in the tumor microenvironment (TME), primarily sourced from cancer-associated fibroblasts and macrophages^{5,10}. Chronic pancreatitis, a key risk factor for PDAC, induces fibrosis, recruits macrophages, and elevates TGFβ^{4,5.}

Clearly, a need exists for new agents which reduce or eliminate EMT-associated therapy resistance in cancer treatment using KRAS inhibitors by targeting the TGFβ pathway, particularly for PDAC, a particularly lethal cancer.

### SUMMARY OF THE INVENTION

In one aspect of the invention, provided herein are methods of treating cancer in a patient, the methods comprising administering an effective amount of a KRAS inhibitor and administering an effective amount a TGFβ inhibitor. In certain embodiments, the KRAS inhibitor is KRASG12D-LODER, Anti-KRAS G12D mTCR PBL(NCI), MRTX-1133, ASP 3082, BI-1701963, HRS-4642, RMC-9805, UA022, DCTY-1102, or DN-022150.

In another aspect of the invention, methods of treating cancer in a patient receiving treatment with a KRASi, or having been previously treated with a KRASi, the methods comprising administering an effective amount of a TGFβ inhibitor are provided herein. In certain embodiments, the TGFβ inhibitor is A77-01, A83-01, AX 12799734, D4476, Distertide, Galunisertib, GW 788388, IN 1130, LY 2109761, R 268712, RepSox, SB431542, SB505124, SB525334, SD208 SM16, or a TGFβ antibody. In certain embodiments, the TGFβ inhibitor is an inhibitor of the canonical TGFβ pathway. In certain embodiments, the TGFβ inhibitor is a SMAD inhibitor, an NFAT5 inhibitor, a S100A4 inhibitor, or an inhibitor of a downstream EMT transcription factor of SMAD.

In certain embodiments, the SMAD inhibitor is pirfenidone, SIS3, Halofuginone, asiaticoside, kartogenin, halofuginonoe hydrochloride, trabedersen sodium, nisevokitug, SRI-011381, trimethylamine N-oxide, oxymatrine, Alantolacone, ponsegromab, halofuginone hydrobromide, hydrochlorothiazide, R-268712, luspatercept, disitertide diammonium, 3,3-dimethyl-1-butanol, trimethylamine N-oxide dihydrate, SY-LB-35, Carotuximab, livmoniplimab, trabedersen, (S,R,S)-AHPC-C2-amide-benzofuranylmethyl-pyridine, chebulinic acid, trimethylamine N-oxide-d₉, SJ000063181, CCT365623 hydrochloride, disitertide TFA, isoviolanthin, mongersen, alk5-in-34, elezanumab, IED 2, or Butaprost. In certain embodiments, the NFAT5 inhibitor is KRN2, KRN5, VIVIT, INCA-6, 1IR-VIVIT TFA, PROTAC BTK Degrader-9, KRM-III, NFATc1-IN-1, cyclosporin D, heraclenin, syringaresinol, Q134R, eudebeiolide B, or gomisin E. In certain embodiments, the S100A4 inhibitor is niclosamide, pentamidine, US-10113, CT070909, or RGC-01-05-18.

In another aspect of the invention, methods of treating cancer in a patient, the method comprising administering an effective amount of a KRAS inhibitor and administering an effective amount a NFAT5 inhibitor are provided herein. In another aspect of the invention, method of treating cancer in a patient receiving treatment with a KRASi, or having been previously treated with a KRASi, the method comprising administering an effective amount of a NFAT5 inhibitor are provided. In certain embodiments, the KRAS inhibitor is KRASG12D-LODER, Anti-KRAS G12D mTCR PBL(NCI), MRTX-1133, ASP 3082, BI-1701963, HRS-4642, RMC-9805, UA022, DCTY-1102, or DN-022150. In another embodiment, the NFAT5 inhibitor is KRN2, KRN5, VIVIT, INCA-6, 11R-VIVIT TFA, PROTAC BTK Degrader-9, KRM-III, NFATc1-IN-1, cyclosporin D, heraclenin, syringaresinol, Q134R, eudebeiolide B, or gomisin E.

In certain embodiments of the methods disclosed herein, the cancer is a therapy resistant and/or an aggressive cancer. In certain embodiments, prior to treatment, the cancer is reinitiated after a previous chemotherapy. In certain embodiments, the previous chemotherapy comprises administration of a KRASi. In certain embodiments of the methods disclosed herein, the cancer is selected from pancreatic ductal adenocarcinoma, acute myeloid leukemia, fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastric cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms'tumor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, glioblastoma, and retinoblastoma.

In certain embodiments of the methods disclosed herein, the methods further comprise administering an additional therapeutic agent. In certain embodiments, the additional therapeutic agent is a compound that acts to block macrophage infiltration and/or acts to re-polarize tumor-associated macrophages to stimulate anti-tumor immunity. In certain embodiments, the additional therapeutic agent is a CCR2 inhibitor or a CSF1R inhibitor or antibody. In certain embodiments, the CCR2 inhibitor is an anti-CCR2 antibody, CCX140, CCX872, PF-04136309 (PF-6309), PF-04178903, INCB-8696, CCX-915, MLN-1202, JNJ-17166864; AZD-2423, INCB-003284, BMS-741672, MK-0812; PF-04634817, CNT0888, or 747 (kaempferol 3-(2,4-di-E-p-coumaroylrhamnoside). In certain embodiments, the CSF1R inhibitor or antibody is pexidartinib, emactuzumab, cabiralizumab, ARRY-382, BLZ945, AJUD010, AMG820, IMC-CS4, JNJ-40346527, PLX5622, or FPA008.

In certain embodiments of the methods disclosed herein, the KRASi and the TGFβi or NFAT5i act synergistically. In certain embodiments, the methods further comprise assessing the patient for a reduction in cancer symptoms.

### BRIEF DESCRIPTION OF THE DRAWINGS

FIG. 1A-1G. Pancreatitis drives KRAS* bypass. (FIG. 1A and FIG. 1B) GSEA analysis of RNA-seq data comparing KRAS* on and off tumors. PDAC cells from iKPC mice were orthotopically transplanted into C57BL/6 mice, followed by dox water administration to maintain KRAS* activation. After 1 wk, four mice continued receiving dox water (ON) while four were switched to normal water to deactivate KRAS* (OFF). 5 days later, tumors were collected for RNA-seq analysis. (FIG. 1A) Top deregulated gene sets in KRAS* OFF or ON tumors by GSEA analysis. (FIG. 1B) Enrichment plots of IFNα response and TGFβ signaling gene sets. (FIG. 1C) Experimental design for inducing chronic pancreatitis in spontaneous PDAC models using CAE at 100 μg/kg. (FIG. 1D) Kaplan-Meier survival analysis comparing mouse groups with KRAS* expression (on dox), KRAS* depletion (off dox), and KRAS* depletion plus CAE treatment (off dox+CAE). (FIG. 1E) Representative histological images illustrating the time-course analysis of malignant lesions and tumors during pancreatitis-induced tumor relapse. H&E, Mason's Trichrome staining, and immunohistochemistry (TIC) were performed. (FIG. 1F) Quantification of IHC signal-positive cells using ImageJ. The percentage of relative area was calculated as 100*(positive cell area)/(total cell area). (FIG. 1G) Quantification of TGFβ IHC signal intensity using ImageJ. The optical density was calculated as log(max intensity/mean intensity), where the max intensity is 255 for 8-bit images. Log-rank (Mantel-Cox) test was used for FIG. 1D. Statistical analysis was performed using one-way ANOVA for FIGS. 1F and 1G. The P values: ns, not significant; *, P<0.05; **, P<0.01, ***, P<0.001; ****, P<0.0001. Error bars represent the median±SEM. All experimental data was verified in at least two independent experiments.

FIG. 2A-2H. Pancreatitis promotes KRASi resistance through TGFβ.(FIG. 2A) Experimental design for inducing pancreatitis in orthotopically transplanted PDAC models. (FIG. 2B) Bioluminescence imaging (BLI) monitoring of tumor formation across different treatment groups: vehicle control+saline+IgG (V), vehicle control+caerulein (CAE, 100 μg/kg)+IgG (C), vehicle control+saline+α-TGFβ neutralizing antibody (250 g per dose, T), vehicle control+CAE+α-TGFβ neutralizing antibody (CT), MRTX1133 (10 mg/kg, BID)+saline+IgG (M), MRTX1133+CAE+IgG (MC), MRTX1133+saline+α-TGFβ neutralizing antibody (MT), and MRTX1133+CAE+α-TGFβ neutralizing antibody (MCT). (FIG. 2C) Images of collected tumors at humane endpoints. (FIG. 2D) Comparison of tumor weights.(FIG. 2E) Comparison of tumor volumes. (FIGS. 2F and 2G) Histological analysis using H&E (FIG. 2F) and IHC (FIG. 2G) staining to characterize tumor morphology and TAMs.(FIG. 2H) Quantification of IHC signal-positive cells from FIG. 2G using ImageJ. Statistical analysis was performed using one-way ANOVA for FIGS. 2D, 2E, and 2H. The P values: ns, not significant; *, P<0.05; **, P<0.01, ***, P<0.001; ****, P<0.0001. Error bars represent the median±SEM. All experimental data was verified in at least two independent experiments.

FIG. 3A-3I. TGFβ pathway activation is required for pancreatitis-induced KRASi resistance. (FIG. 3A) Experimental design for inducing pancreatitis in orthotopically transplanted PDAC models. (FIG. 3B) BLI monitoring tumor formation in comparison groups: MRTX1133 (10 mg/kg, BID)+saline+IgG (M), MRTX1133+CAE (100 μg/kg)+IgG (MC), and MRTX1133+CAE+α-TGFβ neutralizing antibody (250 g per dose, MCT). Collected tumors are shown below. (FIG. 3C-3D) Statistical comparison of tumor weight (FIG. 3C) and tumor volume (FIG. 3D) among the three experimental arms. (FIG. 3E) Western blot analysis of canonical TGFβ pathway activation status in PDAC tissues under different treatments. (FIG. 3F) H&E staining of mouse pancreas following various treatments. (FIG. 3G) IHC staining of TGFB1 in KRAS*-expressing tumors and KRAS*-depleted tumors for 5 days from iKPC mice. (FIG. 3H) Quantification of TGFβ signal intensity in G using ImageJ. OD, optical density. (FIG. 3I) Quantification of relative TGFβ-positive area in G using ImageJ. Statistical analysis for FIGS. 3C, 3D, 3H, and 3I involved one-way ANOVA. The P values: ns, not significant; *, P<0.05; **, P<0.01, ***, P<0.001; ****, P<0.0001. Error bars represent the median±SEM. All experimental data was verified in at least two independent experiments.

FIG. 4A-4L. TGFβ signaling determines PDAC sensitivity to KRAS* targeted therapy. (FIG. 4A) Genetic mutation rates of KRAS, TP53,andSMAD4 in PDAC subtypes, including well, moderately, and poorly differentiated (diff'd), and undifferentiated (undiff'd) subtypes. The QCMG PDAC dataset from cBioPortal was used for the study. (FIG. 4B) Comparison of cancer spheroid formation using three distinct iKPC PDAC cells. (FIG. 4C) The inability of TGFβ to induce KRAS*-independent cancer spheroid formation in some iKPC cell lines. (FIG. 4D) Assessment of Cdkn2a and Cdkn2b expression in various iKPC cell lines. (FIG. 4E) Determination of knockdown efficiency for Cdkn2a and Cdkn2b via qRT-PCR. (FIG. 4F-4G) Comparative analysis of TGFβ-driven, KRAS*-independent cancer spheroid formation following the knockdown of Cdkn2a or Cdkn2b in two distinct KRAS* bypass-deficient iKPC cell lines. (FIG. 4H) Comparison of cancer spheroid formation from KPC PDAC cells upon treatment with G12Di MRTX1133 (0.3 μM), murine recombinant TGFβ (0.5 ng/ml), and TGFβRi SB505124 (3 μM). (FIG. 4I) Cancer spheroid formation from human PDAC MIA PaCa-2 cells upon treatment with KRASG12C inhibitor (G12Ci) ARS-1620 (7.5 μM), human recombinant TGFβ (0.5 ng/ml), and TGFβRi SB505124 (3 μM). (FIG. 4J) Cancer spheroid formation from human PDAC Panc 04.03 upon treatment with G12Di MRTX1133 (0.2 μM), human recombinant TGFβ (0.5 ng/ml), and TGFβRi SB505124 (3 μM). (FIG. 4K) Cancer spheroid formation from human PDAC AsPC-1 upon treatment with G12Di MRTX1133 (0.3 M), human recombinant TGFβ (0.5 ng/ml), and TGFβRi SB505124 (1 μM). (FIG. 4L) Representative images of spheroids from KRAS*-independent escaper tumor cell lines under treatment with TGFβRi SB505124 (3 μM). The control E5 images in FIG. 4L and FIG. 8F were from the same experiment. Statistical analysis was performed using one-way ANOVA for FIG. 4B and FIG. 4E-4K. The P values: ns, not significant; *, P<0.05; **, P<0.01, ***, P<0.001; ****, P<0.0001. Error bars represent the median±SEM. All experimental data was verified in at least three independent experiments.

FIG. 5A-5K. NFAT5 interacts with SMAD3 and SMAD4. (FIG. 5A and FIG. 5B) Western blot analysis to determine the knockdown efficacy of Smad2, Smad3,and Smad4. (FIG. 5C) Examination of TGFβ-driven, KRAS*-independent iKPC cancer spheroid formation after SMADs knockdown compared to the scramble control. (FIG. 5D) Venn diagram illustrating the IP/MS results. Endogenous SMAD2, SMAD3, and SMAD4 were used as baits to pull down proteins in iKPC PDAC cells, with an IgG antibody serving as the negative control. Positive hits are defined as those with an abundance ratio >10 compared with IgG. (FIG. 5E-5F) Validation of protein interactions through co-IP/western blot analysis in mouse iKPC PDAC cells using SMADs and NFAT5 as baits. (FIG. 5G-5I) Validation of NFAT5-SMADs protein interactions through co-IP/western blot analysis in human MIA PaCa-2 PDAC cells. (FIG. 5J) Cell fractionation followed by pulldown of NFAT5 using α-IgG or α-SMAD4 antibody. WCL: whole cell lysate; Cyt: cytosol fraction; Nuc: nuclear fraction. (FIG. 5K) Analysis of NFAT5 and SMADs interaction under different treatments by co-IP/western blots. All experimental data was verified in at least two independent experiments.

FIG. 6A-6K. NFAT5 is upregulated in PDAC. (FIG. 6A) Human tissue microarray (TMA) analysis of NFAT5 during pancreatic disease progression. (FIG. 6B) Quantification of histological scores in chronic pancreatitis (CP), PanIN, and PDAC. (FIG. 6C-6E) Kaplan-Meier survival analysis of PDAC patients with high or low NFAT5 expression in the TCGA PAAD dataset, including overall survival (OS) analysis in the entire cohort (FIG. 6C), the SMAD4 wildtype (wt) cohort (FIG. 6D), and the SMAD4 mutation or deletion (mut/del) cohort (FIG. 6E). (FIG. 6F) IHC staining of NFAT5 in spontaneous tumors from iKPC mice. (FIG. 6G) Quantification of nuclear NFAT5 staining signal intensity in F using ImageJ. (FIG. 6H) IHC staining of NFAT5 in tumor tissues collected from iKPC mice during pancreatitis-induced tumor relapse. (FIG. 6I) Quantification of nuclear NFAT5 staining signal intensity in 6H using ImageJ. (FIG. 6J) IHC staining of NFAT5 in transplanted tumors under treatments with KRASi, CAE, and α-TGFβ neutralizing antibody. (FIG. 6K) Quantification of nuclear NFAT5 staining signal intensity in J using ImageJ. One-way ANOVA was used for statistical analysis for FIGS. 6G, 6I, and 6K; the Chi-square test was performed for B; the Log-rank (Mantel-Cox) test was used for FIG. 6C-6E. The P values: ns, not significant; *, P<0.05; **, P<0.01, ***, P<0.001; ****, P<0.0001. Error bars represent the median±SEM. All experimental data was verified in at least two independent experiments.

FIG. 7A-7J. NFAT5 is essential for TGFβ-driven KRAS* bypass. (FIG. 7A) Nfat5 expression in primary KRAS*-expressing PDAC tumors (iKPC), KRAS*-reactivated escaper tumors (KRAS+E), and KRAS*-independent escaper tumors (KRAS-E) from iKPC mice (left). The same dataset was reanalyzed to indicate Nfat5 expression in different subtypes of escaper tumors from iKPC mice (right), including classical, hybrid, and QM escapers. (FIGS. 7B and 7C) Knockdown efficiency of Nfat5 in iKPC cells assessed by RT-PCR (FIG. 7B) and western blot (FIG. 7C) analysis. (FIG. 7D-7E) Cancer spheroid formation assay comparing Nfat5 knockdown to the vehicle control in KRAS*-expressing iKPC PDAC cells and TGFβ-driven KRAS* bypass. Three different iKPC cell lines were used for the study. (FIG. 7F) Experimental design to assess the anti-tumor effect of Nfat5 knockdown in combination with G12Di in vivo. (FIG. 7G) Comparison of tumor growth between the scramble control and Nfat5 knockdown under treatment with G12Di MRTX1133 (10 mg/kg, QD) or vehicle control. Tumors were collected on day 21. (FIG. 7H) Tumor characterization by H&E staining. (FIG. 7I) Characterization of tumors from G by IHC staining. (FIG. 7J) Quantification of Ki67+ cell number per 10× view from I by ImageJ. One-way ANOVA was used for statistical analysis for FIGS. 7A, 7B, 7D, 7E, and 7J; the unpaired, two-tailed t test was used for FIG. 7G at the time point of tumor collection. The P values: ns, not significant; *, P<0.05; **, P<0.01, ***, P<0.001; ****, P<0.0001. Error bars represent the median±SEM. All experimental data was verified in at least two independent experiments.

FIG. 8A-8S. NFAT5 inhibition mitigates KRAS* targeted therapy resistance. (FIG. 8A) Western blot analysis to determine the dose-dependent inhibition of NFAT5 expression by chemical compound KRN2. (FIG. 8B) Comparison of cancer spheroid formation under the treatment of different combinations of dox, TGFβ (0.5 ng/ml), and KRN2 (1 μM) in three distinct iKPC PDAC cell lines. (FIG. 8C) Cancer spheroid formation assay to assess the combination effect of G12Di MRTX1133 (0.03 μM) and KRN2 (1 μM) in KPC PDAC cells. (FIG. 8D) Cancer spheroid formation assay to determine the combination effect of G12Ci ARS-1620 (5 μM) and KRN2 (0.3 μM) in human PDAC MIA PaCa-2 cells. (FIG. 8E) Cancer spheroid formation assay to determine the combination effect of G12Di MRTX1133 (0.3 μM) and KRN2 (0.3 μM) in human PDAC AsPC-1 cells. (FIG. 8F) Comparison of cancer spheroid formation under treatment of DMSO control, TGFβRi SB505124 (3 μM), and KRN2 (1 μM) in three KRAS*-independent escaper tumor cell lines from iKPC mice without CAE treatment. The control E5 images in FIG. 4L and FIG. 8F were from the same experiment. (FIG. 8G) Comparison of cancer spheroid formation under treatment of DMSO control, TGFβRi SB505124 (3 μM), and KRN2 (1 μM) in three KRAS*-independent escaper tumor cell lines from CAE-treated iKPC mice. (FIG. 8H) Experimental design to evaluate the anti-tumor effect of KRN2 (3 mg/kg, QD) monotherapy and its combination with G12Di MRTX1133 (10 mg/kg, QD) in vivo. (FIG. 8I) BLI imaging to monitor tumor formation. (FIG. 8J) Kaplan-Meier survival analysis. OS, overall survival. (FIG. 8K) Measurement of mouse body weight along treatments. (FIG. 8L) Tumor characterization by H&E staining. (FIG. 8M) Schematic of the experimental design to assess the combined inhibition of KRAS and NFAT5 in the MIA PaCa-2 orthotopic xenograft model. Tumor-bearing mice were under treatment of vehicle control, G12Ci MRTX849 (100 mg/kg, QD), KRN2 (3 mg/kg, QD), and the combination (combo). (FIG. 8N) Measurement of mouse body weight with and without treatments. (FIG. 8O) Comparison of tumor weight and size on day 28. (FIG. 8P) Analysis of escaper tumor growth comparing treatment of vehicle control and KRN2 (3 mg/kg, QD). KRAS*-independent escaper tumor cells E725 were transplanted into nude mice subcutaneously. Tumors were collected and imaged on day 28.(FIG. 8Q) Comparison of tumor growth by BLI under treatments: MRTX1133 (10 mg/kg, BID)+saline+vehicle (M), MRTX1133+CAE (100 μg/kg)+vehicle (MC), and MRTX1133+CAE+KRN2 (3 mg/kg, QD, MCK). The KPC PDAC cells (1860) were orthotopically transplanted in immunocompetent mice. Tumors were collected on day 21. (FIG. 8R-8S) Statistical comparison of tumor volume (FIG. 8R) and tumor weight (FIG. 8S) among the three experimental arms. One-way ANOVA was used for statistical analysis for FIGS. 8B-8G, 8R, and 8S; the unpaired, two-tailed t test was used for FIG. 8O-8P at the time point of tumor collection. The P values: ns, not significant; *, P<0.05; **, P<0.01, ***, P<0.001; ****, P<0.0001. Error bars represent the median±SEM. All experimental data was verified in at least two independent experiments.

FIG. 9A-9U. S100A4 is a direct target of the NFAT5-SMADs complex. (FIG. 9A) Summary of RNA-seq analysis to identify candidate targets of the NFAT5-SMADs complex. (FIG. 9B) GSEA analysis to identify the loss of the EMT gene signature after the inhibition or knockdown of NFAT5. (FIG. 9C) Intersection of RNA-seq datasets to identify 99 candidate genes potentially activated by the NFAT5-SMADs complex. (FIG. 9D) Expression profile of gene candidates in primary and escaper PDAC tumor cells from iKPC mice. (FIG. 9E) Comparison of S100a4 expression in primary and escaper PDAC tumors based on KRAS reactivation status (left) and tumor subtypes (right). (FIG. 9F) Summary of ChIP-seq data revealing genes with proximal promoters bound by NFAT5 and SMADs. (FIG. 9G) Schematic representation of the NFAT5-SMADs interaction. (FIG. 9H) Overlapping genes between the 99 candidates from RNA-seq and 2,582 genes from ChIP-seq. (FIG. 9I) IHC staining of S100A4 in tumors during pancreatitis-driven KRAS* bypass and escaper tumors. (FIG. 9J) Quantification of relative S100A4 signal-positive area in I using ImageJ. (FIG. 9K) IHC staining of S100A4 in transplanted tumors under treatments with KRASi, CAE, and α-TGFβ neutralizing antibody. (FIG. 9L) Quantification of relative S100A4 signal-positive area in K using ImageJ. (FIG. 9M) Kaplan-Meier survival analysis of PDAC patients based on high or low S100A4 expression in TCGA PAAD dataset.(FIG. 9N) Expression changes of S100a4 after treatments with dox or TGFβ, following knockdown of Nfat5 or Smad2/3/4. (FIG. 9O) Binding of NFAT5 and SMADs at the S100a4 promoter. (FIG. 9P) NFAT5-SMADs binding comparison at the S100a4 promoter in Nfat5 wildtype and knockdown iKPC cells. (FIG. 9Q-9R) Comparison of luciferase activity driven by full length (FL) of or truncated S100a4 promoter. (FIG. 9S) Comparison of S100a4 activation under treatment of NFAT5i KRN2 (1 μM) or TGFβRi SB505124 (3 μM) by luciferase reporter assay under the control of the S100a4 promoter (FL). (FIG. 9T) Western blot analysis of S100A4 expression regulated by TGFβ in iKPC spheroids.(FIG. 9U) Western blot analysis of EMT TF expression after Nfat5 or S100a4 knockdown. Statistical analysis for FIGS. 9E, 9J, 9L, 9N, 9P, 9R, and 9S involved one-way ANOVA; the Log-rank (Mantel-Cox) test was used for FIG. 9M. The P values: ns, not significant; *, P<0.05; **, P<0.01, ***, P<0.001; ****, P<0.0001. Error bars represent the median±SEM. All experimental data was verified in at least two independent experiments.

FIG. 10A-10E. Discovery of S100a4 as a direct target of the NFAT5-SMADs complex. (FIG. 10A-10B) GSEA to unveil deregulated gene sets by Smad3 and Nfat5 knockdown (FIG. 10A) and KRN2 (FIG. 10B). (FIG. 10C) GSEA to show the shared deregulated genes following Smad3 and Nfat5 knockdown. (FIG. 10D) Overview of DNA binding regions for NFAT5, SMAD2, SMAD3, and SMAD4 in the mouse genome as determined by ChIP-seq. (FIG. 10E) GSEA to indicate genes bound by the NFAT5-SMADs complex (left) and genes exclusively bound by SMADs (right).

FIG. 11A-11C. The NFAT5-SMADs complex regulates canonical TGFβ pathway targets. (FIG. 11A) Predicted DNA binding motifs for NFAT5 and SMADs, with predicted binding sites on the S100a4 (S100A4) gene region in the mouse (human) genome. (FIG. 11B) Expression levels of EMT TFs following Nfat5 knockdown or inhibition, and the binding of NFAT5 and SMADs to the DNA regions of EMT TFs. (FIG. 11C) The binding of NFAT5 and SMADs at the Nfat5 promoter.

FIG. 12A-12G. S100A4 is required for KRAS* bypass driven by the TGFβ-NFAT5 axis. (FIG. 12A) Knockdown efficiency of S100A4 in iKPC cells by western blot. (FIG. 12B) Examination of pathway activation after knockdown of S100a4 in iKPC cells by western blot. (FIG. 12C) TGFβ-driven, KRAS*-independent cancer spheroid formation comparison between S100a4 wildtype and knockdown in three distinct iKPC cell lines. (FIG. 12D) Tumor growth analysis of subcutaneously transplanted S100a4 wildtype and knockdown (KD) iKPC cells under treatment of vehicle control or G12Di MRTX1133 (10 mg/kg, QD). (FIG. 12E) Tumor characterization by H&E staining. (FIGS. 12F and 12G) Rescue of TGFβ-driven, KRAS*-independent cancer spheroid formation by S100A4 after NFAT5 inhibition (KRN2, 1 μM) (FIG. 12F) and after knockdown of S100a4 and Nfat5 (FIG. 12G). Statistical analysis for FIGS. 12C and 12F involved one-way ANOVA; the unpaired, two-tailed t test was used for FIG. 12G and for 12D at the time point of tumor collection. The P values: ns, not significant; *, P<0.05; **, P<0.01, ***, P<0.001; ****, P<0.0001. Error bars represent the median±SEM. All experimental data was verified in at least two independent experiments.

FIG. 13A-13Q. Macrophages promote KRAS* bypass by providing paracrine S100A4. (FIG. 13A) Single-cell RNA-seq analysis to reveal S100a4 expression in tumors collected from KPC and iKPC mice, treated with MRTX1133 (Ki, 10 mg/kg, BID) or with KRAS off for 5 days. (FIG. 13B) Differential expression of Tgfb1 and S100a4 in bone marrow-derived macrophages (mBMDMs) compared to iKPC cells. (FIG. 13C) Assessment of Tgfb1 and S100a4 expression in mBMDMs post-treatment with M0 inducer (M-CSF), M1 inducer (LPS+IFNγ), M2 inducer (IL-4), tumor-conditioned medium collected from KPC cells (CM), and tumor-conditioned medium collected from KRAS-inhibited KPC cells+KRASi CM). (FIG. 13D) S100a4 expression in mBMDMs under treatment with TGFβ (0.5 ng/ml), NFAT5i KRN2 (1 μM), or TGFβRi SB505124 (3 μM). (FIG. 13E) IHC staining of F4/80 and S100A4 in transplanted tumors with wildtype or Nfat5 knockdown after MRTX1133 treatment. (FIG. 13F) Quantification of relative F4/80 signal-positive area in FIG. 13E using ImageJ. (FIG. 13G) Quantification of S100A4 high stroma cell number in FIG. 13E using ImageJ. (FIG. 13H) IHC staining of F4/80 in transplanted tumors post MRTX1133 and KRN2 treatment. (FIG. 13I) Quantification of relative F4/80 signal-positive area in FIG. 13H using ImageJ. (FIG. 13J) TGFβ-driven, KRAS*-independent cancer spheroid formation with or without co-culture of mBMDMs (Mφs, 30,000 cells/well) after S100a4 knockout. (FIG. 13K) KRAS*-independent cancer spheroid for-mation in co-culture with mBMDMs. (FIG. 13L) KRAS*-independent, Nfat5-knockdown cancer spheroid formation in co-culture with mBMDMs. (FIG. 13M) KRAS*-independent cancer spheroid formation in co-culture with mBMDMs under treatment of S100A4 and TGFβ neutralizing antibodies. The concentrations for IgG isotype control, α-S100A4 antibody and α-TGFβ antibody were 10, 5, and g/ml, respectively. (FIG. 13N) Overlapping genes between RNA-seq datasets and secretome database. (FIG. 13O) Expression changes of Ccl2 in iKPC cells post TGFβ treatment, after Smad2/3/4 knockdown, and Nfat5 knockdown. (FIG. 13P) NFAT5 and SMADs binding at the Cc12 promoter. (FIG. 13Q) Ccl2 expression in primary and escaper PDAC tumors based on KRAS reactivation status (left) and tumor subtypes (right). Statistical analysis for FIGS. 13C, 13D, 13F, 13G, 13I, 13K-13M, 130, and 13Q involved one-way ANOVA; the unpaired, two-tailed t test was used for FIG. 13B and FIG. 13J. The P values: ns, not significant; *, P<0.05; **, P<0.01, ***, P<0.001; ****, P<0.0001. Error bars represent the median±SEM. All experimental data was verified in at least two independent experiments.

FIG. 14. Schematic representation of intercellular crosstalk promoting KRAS* bypass.

### DETAILED DESCRIPTION OF THE INVENTION

Oncogenic KRAS is now considered a druggable target; however, multiple mechanisms contribute to the development of resistance to KRAS-targeted therapy¹. A significant factor in therapy resistance is the alteration in cell state or cellular plasticity, exemplified by the epithelial-to-mesenchymal transition (EMT) phenotype². In pancreatic ductal adenocarcinoma (PDAC), the negative correlation between addiction to oncogenic KRAS signaling and EMT has been observed³, yet the role of cell plasticity and its underlying mechanisms in governing resistance remain unclear. Chronic pancreatitis is a recognized risk factor for pancreatic tumorigenesis, inducing inflammation and fibrosis while elevating TGFβ levels in the tumor microenvironment (TME)^{4,5.}

In this study, we demonstrate that the experimental induction of chronic pancreatitis promotes resistance to KRAS-targeted therapy in a TGFβ-dependent manner. Our findings reveal that the pivotal EMT driver, TGFβ, facilitates KRAS bypass in PDAC through the nuclear factor NFAT5. NFAT5 interacts with canonical TGFβ factors SMAD3 and SMAD4, inducing EMT and therapy resistance via the transcriptional activation of a chaperone protein and an extracellular matrix regulator, S100A4. Despite the direct DNA binding of SMAD3 and SMAD4, their binding strength is weak, necessitating co-factors for the activation of various gene targets¹⁵. Notably, the nuclear factor of activated T cells 5 (NFAT5) is identified as an interactor of SMAD3 and SMAD4 and a critical mediator of TGFβ-driven KRAS* independency in PDAC.

Furthermore, our investigation indicates that TGFβ stimulates PDAC cells to secrete the chemokine CCL2, recruiting circulating macrophages. These macrophages, in turn, support PDAC cells to bypass KRAS through paracrine TGFβ and S100A4. Overall, our results elucidate the regulatory role of canonical TGFβ signaling in EMT-associated KRAS-targeted therapy resistance and identify NFAT5 as a chemically druggable target. Targeting NFAT5 could disrupt this regulatory network, offering a potential avenue for preventing the resistance process in PDAC.

Belonging to the Rel family, NFAT5 possesses a Rel-homology domain (RHD) for DNA binding¹⁶. Functional and mechanistic studies reveal that the NFAT5-SMAD3/4 complex binds to the promoter of S100 Calcium Binding Protein A4 (S100A4) to activate its transcription, thereby supporting KRAS* bypass. Additionally, TGFβ pathway activation recruits S100A4-positive macrophages. Inhibition of NFAT5 suppresses S100A4 expression in both tumor cells and macrophages, preventing EMT-associated KRASi resistance and impairing escaper tumor maintenance.

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

The terms “about” or “approximately” in the context of numerical values and ranges refers to values or ranges that approximate or are close to the recited values or ranges such that the embodiment may perform as intended, such as having a desired amount of nucleic acids or polypeptides in a reaction mixture, as is apparent to the skilled person from the teachings contained herein. In some embodiments, about means plus or minus 10% of a numerical amount.

Furthermore, a compound “selected from the group consisting of” refers to one or more of the compounds in the list that follows, including mixtures (i.e., combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

### Therapeutic Compounds

The inhibitors described herein may be used, alone or in combination, in methods for treating cancer.

The terms “inhibition” or “inhibit” refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, “reduce” or “inhibit” refers to the ability to cause an overall decrease of 20% or greater. In another embodiment, “reduce” or “inhibit” refers to the ability to cause an overall decrease of 50% or greater. In yet another embodiment, “reduce” or “inhibit” refers to the ability to cause an overall decrease of 75%, 85%, 90%, 95%, or greater. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”.

The term “inhibitor” refers to an agent that slows down or prevents a particular chemical reaction, signaling pathway or other process, or that reduces the activity of a particular reactant, catalyst, or enzyme.

In certain embodiments, the compounds described herein act to inhibit KRAS and are useful as therapeutic or prophylactic therapy when such inhibition is desired, e.g., for the treatment of cancer. Unless otherwise indicated, when uses of the compounds of the present disclosure are described herein, it is to be understood that such compounds may be in the form of a composition (e.g., a pharmaceutical composition).

The term “KRAS” or “Kristen RAt Sarcoma virus” refers to a gene that makes a protein that is involved in cell signaling pathways that control cell growth, cell maturation, and cell death. The natural, unchanged form of the gene is called wild-type KRAS. Mutated (changed) forms of the KRAS gene have been found in some types of cancer, including non-small cell lung cancer, colorectal cancer, and pancreatic cancer. These changes may cause cancer cells to grow and spread in the body.

The term “KRAS inhibitor” or “KRASi” refers to any compound which decreases expression of KRAS or levels of a KRAS proteins in a subject, or any compound which binds to a KRAS protein or KRAS receptor and disrupts the interaction of ligand with any of the receptors. Exemplary KRAS inhibitors include, without limitation, an anti-KRAS antibody, KRASG12D-LODER, Anti-KRAS G12D mTCR PBL(NCI), MRTX-1133, ASP 3082, BI-1701963, HRS-4642, RMC-9805, UA022, DCTY-1102, DN-022150.

In certain embodiments, the compounds described herein act to inhibit the TGFβ pathway and are useful as therapeutic or prophylactic therapy when such inhibition is desired, e.g., for the treatment of cancer. Unless otherwise indicated, when uses of the compounds of the present disclosure are described herein, it is to be understood that such compounds may be in the form of a composition (e.g., a pharmaceutical composition).

The term “TGFβ inhibitor” or “TGFβi” refers to any compound which decreases expression of TGFβ or levels of TGFβ proteins in a subject, or any compound which binds to TGFβ or TGFβ receptor and disrupts the interaction of ligand (TGFb) with any of the TGFβ receptors (Type I, Type II and/or Type III). Exemplary TGFβ inhibitors include, without limitation, anti-TGFβ antibodies, A77-01, A83-01, AX 12799734, D4476, Distertide, Galunisertib, GW 788388, IN 1130, LY 2109761, R 268712, RepSox, SB431542, SB505124, SB525334, SD208 SM16, and TGFβ antibodies.

In certain embodiments, the TGFβi is an inhibitor of the canonical TGFβ pathway. The canonical TGFβ pathway refers to modulating the TGFβ pathway using a SMAD-dependent mechanism.

In certain embodiments, the compounds described herein act to inhibit the SMAD and are useful as therapeutic or prophylactic therapy when such inhibition is desired, e.g., for the treatment of cancer. Unless otherwise indicated, when uses of the compounds of the present disclosure are described herein, it is to be understood that such compounds may be in the form of a composition (e.g., a pharmaceutical composition).

The term “SMAD inhibitor” or “SMADi” refers to any compound which decreases expression of a SMAD protein or levels of a SMAD protein in a subject, or any compound which binds to a SMAD protein or SMAD receptor and disrupts the interaction of ligand with any of the receptors. Exemplary SMAD inhibitor include without limitation inhibitors of any SMAD protein including SMAD1, SMAD2, SMAD3, SMAD4, SMAD5, and/or SMAD8/9.

In certain embodiments, the compounds described herein act to inhibit the canonical SMAD pathway and are useful as therapeutic or prophylactic therapy when such inhibition is desired, e.g., for the treatment of cancer. Unless otherwise indicated, when uses of the compounds of the present disclosure are described herein, it is to be understood that such compounds may be in the form of a composition (e.g., a pharmaceutical composition).

In certain embodiments the canonical SMAD inhibitors inhibits SMAD3 and/or SMAD4. Exemplary SMAD3 and SMAD4 inhibitors include, without limitation, pirfenidone, SIS3, Halofuginone, asiaticoside, kartogenin, halofuginonoe hydrochloride, trabedersen sodium, nisevokitug, SRI-011381, trimethylamine N-oxide, oxymatrine, Alantolacone, ponsegromab, halofuginone hydrobromide, hydrochlorothiazide, R-268712, luspatercept, disitertide diammonium, 3,3-dimethyl-1-butanol, trimethylamine N-oxide dihydrate, SY-LB-35, Carotuximab, livmoniplimab, trabedersen, (S,R,S)-AHPC-C2-amide-benzofuranylmethyl-pyridine, chebulinic acid, trimethylamine N-oxide-d₉, SJ000063181, CCT365623 hydrochloride, disitertide TFA, isoviolanthin, mongersen, alk5-in-34, elezanumab, IED 2, and Butaprost.

In certain embodiments, the compounds described herein act to inhibit the downstream EMT transcription factors (TF) of SMAD and are useful as therapeutic or prophylactic therapy when such inhibition is desired, e.g., for the treatment of cancer. Unless otherwise indicated, when uses of the compounds of the present disclosure are described herein, it is to be understood that such compounds may be in the form of a composition (e.g., a pharmaceutical composition).

Downstream EMT TF of SMAD include without limitation, NFAT5, SNAI1, SNAI2, ZEB1, ZEB2, TWIST1, TWIST2. Inhibitors directed to any one of these TF may be used in the methods discussed below.

In certain embodiments, the compounds described herein act to inhibit NFAT5 and are useful as therapeutic or prophylactic therapy when such inhibition is desired, e.g., for the treatment of cancer. Unless otherwise indicated, when uses of the compounds of the present disclosure are described herein, it is to be understood that such compounds may be in the form of a composition (e.g., a pharmaceutical composition).

The term “NFAT5 inhibitor” or “NFAT5i” refers to any compound which decreases expression of NFAT5 or levels of a NFAT5 protein in a subject, or any compound which binds to a NFAT5 protein or NFAT5 receptor and disrupts the interaction of ligand with any of the receptors. Exemplary NFAT5 inhibitors include, without limitation, anti-NFAT5 antibodies KRN2, KRN5, VIVIT, INCA-6, 11R-VIVIT TFA, PROTAC BTK Degrader-9, KRM-III, NFATc1-IN-1, cyclosporin D, heraclenin, syringaresinol, Q134R, eudebeiolide B, and gomisin E.

In certain embodiments, the compounds described herein act to inhibit S100A4 and are useful as therapeutic or prophylactic therapy when such inhibition is desired, e.g., for the treatment of cancer. Unless otherwise indicated, when uses of the compounds of the present disclosure are described herein, it is to be understood that such compounds may be in the form of a composition (e.g., a pharmaceutical composition).

The term “S100A4 inhibitor” or “S100A4i” refers to any compound which decreases expression of S100A4 or levels of a S100A4 protein in a subject, or any compound which binds to a S100A4 protein or S100A4 receptor and disrupts the interaction of ligand with any of the receptors. Exemplary S100A4 inhibitors include, without limitation, niclosamide, pentamidine, US-10113, CT070909, RGC-01-05-18, and S100A4 neutralizing antibodies such as clone 6B12 from Arxx Therapeutics.

In certain embodiments, the compounds described herein act to block macrophage infiltration and are useful as therapeutic or prophylactic therapy when such inhibition is desired, e.g., for the treatment of cancer. Unless otherwise indicated, when uses of the compounds of the present disclosure are described herein, it is to be understood that such compounds may be in the form of a composition (e.g., a pharmaceutical composition).

In certain embodiments, the compound that acts to block macrophage infiltration is a CCR2 inhibitor. The term “CCR2 inhibitor” or “CCR2i” refers to any compound which decreases expression of CCR2 or levels of a CCR2 protein in a subject, or any compound which binds to a CCR2 protein or CCR2 receptor and disrupts the interaction of ligand with any of the receptors. Exemplary CCR2 inhibitors include, without limitation, anti-CCR2 antibodies CCX140, CCX872, PF-04136309 (PF-6309), PF-04178903, INCB-8696, CCX-915, MLN-1202, JNJ-17166864; AZD-2423, INCB-003284, BMS-741672, MK-0812; PF-04634817, CNT0888, and 747 (kaempferol 3-(2,4-di-E-p-coumaroylrhamnoside).

In certain embodiments, the compounds described herein act to re-polarize tumor-associated macrophages (TAMs) to stimulate anti-tumor immunity and are useful as therapeutic or prophylactic therapy when such inhibition is desired, e.g., for the treatment of cancer. Unless otherwise indicated, when uses of the compounds of the present disclosure are described herein, it is to be understood that such compounds may be in the form of a composition (e.g., a pharmaceutical composition).

In certain embodiments, the compound that acts to re-polarize TAMs to stimulate anti-tumor immunity is a CSF1R inhibitor or antibody. The term “CSF1R inhibitor” or “CSF1Ri” refers to any compound which decreases expression of CSF1R or levels of a CSF1R protein in a subject, or any compound which binds to a CSF1R protein or CSF1R receptor and disrupts the interaction of ligand with any of the receptors. Exemplary CSF1R inhibitors include, without limitation, pexidartinib, emactuzumab, cabiralizumab, ARRY-382, BLZ945, AJUDO10, AMG820, IMC-CS4, JNJ-40346527, PLX5622, and FPA008.

### Methods of Treatment and Administration

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, or stabilize, a pathological condition or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. It is understood that treatment, while intended to cure, ameliorate, or stabilize, a disease, pathological condition, or disorder, need not actually result in the cure, ameliorization, or stabilization. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

As used herein, the terms “tumor”, “tumor growth” or “tumor tissue” can be used interchangeably, and refer to an abnormal growth of tissue resulting from uncontrolled progressive multiplication of cells and serving no physiological function. In certain embodiments, the tumor is a pancreatic ductal adenocarcinoma (PDAC). A solid tumor can be malignant, e.g. tending to metastasize and being life threatening, or benign. Examples of solid tumors that can be treated or prevented according to a method of the present invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastic cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' mor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, glioblastoma, and retinoblastoma.

The phrase “KRASi resistant cancer” refers to a cancer or tumor in a patient that is able to survive a KRASi that is able to kill or weaken cancers that are not resistant. KRASi resistance may be present before treatment is given or may occur during or after treatment with the drug. In cancer treatment, there are many things that may cause resistance to anticancer drugs, such as KRAS inhibitors. For example, DNA changes or other genetic changes may change the way the KRASi gets into the cancer cells or the way the KRASi is broken down within the cancer cells. KRASi resistance can lead to cancer treatment not working or to the cancer coming back. KRASi resistance can be determined by comparing the effect of a KRASi on a resistant cancer and on a control cancer.

The term “drug response” as used herein, means any biological response in an organism that is the result of exposure to the drug. Drug responses can be favorable, such as when a patient's disease is eradicated by treatment with the drug, or unfavorable, such as when a patient enters a coma upon treatment with a drug.

The term “synergy” or “synergistic” refers to the interaction or cooperation of two or more substances, or other agents to produce a combined effect greater than the sum of their separate effects.

In certain embodiments, treatment using two or more substances improves the therapy using either one of the substances alone, by maximizing efficacy, reducing toxicity, and addressing interpatient variability, as well as delaying and/or overcoming innate or acquired resistance.

Provided herein are methods of treatment of cancer including Pancreatic ductal adenocarcinoma (PDAC). In certain embodiments, the cancer is a resistant to treatment with a chemotherapeutic agent. In certain embodiments, the cancer is resistant to treatment with a KRAS inhibitor. The methods include administration of an effective amount of at least one KRAS inhibitor and at least one agent that inhibits the TGFβ pathway to a subject in need thereof. In certain embodiments the agent that inhibits the TGFβ pathway is selected from a TGFβ inhibitor, a SMAD inhibitor, a SMAD3 or SMAD4 inhibitor, a downstream inhibitor of a SMAD EMT transcription factor, a NFAT5 inhibitor, a S100A4 inhibitor. In certain embodiments, more than one inhibitor of the TGFβ pathway are administered. For example, in certain embodiments, a NFAT5 inhibitor and a S100A4 inhibitor are administered. In some embodiments, the symptoms of the cancer are reduced, as compared to a control.

Also provided herein are methods of treating cancer comprising administering a KRAS inhibitor and a NFAT5 inhibitor. In some embodiments, the symptoms of the cancer are reduced, as compared to a control.

Also provided herein are methods of treating cancer comprising administering a KRAS inhibitor and a S100A4 inhibitor. In some embodiments, the symptoms of the cancer are reduced, as compared to a control.

In certain embodiments, the methods include administration of at least one additional chemotherapeutic agent or chemotherapy. In certain embodiments, the additional chemotherapeutic agent or chemotherapy is a compound that blocks macrophage infiltration or a compound that acts to re-polarize TAMs to stimulate anti-tumor immunity. In certain embodiments, the compound that blocks macrophage infiltration is a CCR2 inhibitor. In certain embodiments, the compound that acts to re-polarize TAMs to stimulate anti-tumor immunity is a CSF1R inhibitor or antibody.

Other cancer treatments which may be used in combination with the compositions provided herein include, without limitation, surgery, chemotherapy, radiation therapy, targeted therapy, immunotherapy, and/or hormone therapy. Known chemotherapeutic agents include, without limitation alkylating agent, anti-metabolic antineoplastic agent, anti-tumor antibiotic, anti-tumor botanical, platinum compound antineoplastic agent, hormonal balance antineoplastic agent, and miscellaneous antineoplastic agent, wherein therapeutical agent used in said targeted therapy is selected from the group consisting of rituximab, bevacizumab, trastuzumab, imatinib, dinoxetine, cetuximab, nilotinib, and sorafenib, wherein therapeutical agent used in said immunotherapy is selected from the group consisting of PD-1 inhibitor, PD-L1 inhibitor and CTLA4 inhibitor; more preferably, said alkylating agent is selected from the group consisting of cyclophosphamide, ifosfamide and thiotepa, said anti-metabolic antineoplastic agent is selected from the group consisting of methotrexate, mercaptopurine, fluorouracil and cytarabine, said anti-tumor antibiotic is selected from the group consisting of bleomycin, daunorubicin, actinomycin D, mitomycin, doxorubicin and mitoxantrone, said anti-tumor botanical is selected from the group consisting of vincristine, etoposide, teniposide, paclitaxel and docetaxel, said platinum compound antineoplastic agent is selected from the group consisting of cisplatin, carboplatin and oxaliplatin, said hormone balance antineoplastic agent is selected from the group consisting of leuprolide, tamoxifen, flutamide and formestane, said miscellaneous antineoplastic agent is arsenic trioxide.

In certain embodiments, another additional chemotherapeutic agent is administered. The additional therapy is an immune checkpoint blockade therapy, such as anti-PD-L1 antibodies. In certain embodiment the additional therapy is surgery, chemotherapy, radiation therapy, targeted therapy, immunotherapy, and/or hormone therapy. In certain embodiments, the chemotherapeutic agents is alkylating agent, anti-metabolic antineoplastic agent, anti-tumor antibiotic, anti-tumor botanical, platinum compound antineoplastic agent, hormonal balance antineoplastic agent, and miscellaneous antineoplastic agent, wherein therapeutical agent used in said targeted therapy is selected from the group consisting of rituximab, bevacizumab, trastuzumab, imatinib, dinoxetine, cetuximab, nilotinib, and sorafenib, wherein therapeutical agent used in said immunotherapy is selected from the group consisting of PD-1 inhibitor, PD-L1 inhibitor and CTLA4 inhibitor; more preferably, said alkylating agent is selected from the group consisting of cyclophosphamide, ifosfamide and thiotepa, said anti-metabolic antineoplastic agent is selected from the group consisting of methotrexate, mercaptopurine, fluorouracil and cytarabine, said anti-tumor antibiotic is selected from the group consisting of bleomycin, daunorubicin, actinomycin D, mitomycin, doxorubicin and mitoxantrone, said anti-tumor botanical is selected from the group consisting of vincristine, etoposide, teniposide, paclitaxel and docetaxel, said platinum compound antineoplastic agent is selected from the group consisting of cisplatin, carboplatin and oxaliplatin, said hormone balance antineoplastic agent is selected from the group consisting of leuprolide, tamoxifen, flutamide and formestane, said miscellaneous antineoplastic agent is arsenic trioxide.

In certain embodiments, the method of treatment effectively suppresses symptoms associated with cancer. Symptoms of vary according to the location and type of cancer being treated. In certain embodiments, symptoms of cancer include, fatigue, weight loss, lumps, pain coughing, wheezing, new or unusual growth, discoloration, and no symptoms at all. In certain embodiments, the treatment reduces the risk of relapse. In the context of a cancer, treatment or inhibition may be assessed by inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS, as used herein means the time from treatment onset until death from any cause. TTP, as used herein refers to the time from treatment onset until tumor progression; TTP does not include deaths. Time to Remission (TTR) as used herein means the time from treatment onset until remission, for example, complete or partial remission. As used herein, PFS means the time from treatment onset until tumor progression or death. In one embodiment, PFS rates will be computed using the Kaplan-Meier estimates. Event-free survival (EFS) means the time from study entry until any treatment failure, including disease progression, treatment discontinuation for any reason, or death. Relapse-free survival (RFS) means the length of time after the treatment ends that the patient survives without any signs or symptoms of that cancer. Overall response rate (ORR) means the sum of the percentage of patients who achieve complete and partial responses. Complete remission rate (CRR) refers to the percentage of patients achieving complete remission (CR). Duration of response (DoR) is the time from achieving a response until relapse or disease progression. Duration of remission is the time from achieving remission, for example, complete or partial remission, until relapse. In the extreme, “complete inhibition”, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

The compounds described herein can be formulated for enteral, parenteral, topical, or systemic administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. Typical carriers and conventional methods of preparing pharmaceutical compositions that can be used in conjunction with the preparation of formulations of the compounds are known by those skilled in the art. These compositions can take the form of solutions, suspensions, emulsion, tablets, pills, capsules, powders, sustained-release formulations and the like.

The compounds described herein can be formulated for parenteral administration. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

For intravenous administration, the compositions may be packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above.

The compounds described herein can be administered in an effective amount to a subject that is in need of alleviation or amelioration from one or more symptoms associated with tumor growth.

The exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation. The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The compositions are administered in an effective amount and for a period of time effect to reduce one or more symptoms associated with the disease to be treated. It should be understood that the “effective amount” for a composition having anti-cancer cell proliferation properties may vary. In one embodiment an effective amount includes without limitation about 0.001 to about 25 mg/kg subject body weight. In one embodiment, the range of effective amount is 0.001 to 0.01 mg/kg body weight. In another embodiment, the range of effective amount is 0.001 to 0.1 mg/kg body weight. In another embodiment, the range of effective amount is 0.001 to 1 mg/kg body weight. In another embodiment, the range of effective amount is 0.001 to 10 mg/kg body weight. In another embodiment, the range of effective amount is 0.001 to 20 mg/kg body weight. In another embodiment, the range of effective amount is 0.01 to 25 mg/kg body weight. In another embodiment, the range of effective amount is 0.01 to 0.1 mg/kg body weight. In another embodiment, the range of effective amount is 0.01 to 1 mg/kg body weight. In another embodiment, the range of effective amount is 0.01 to 10 mg/kg body weight. In another embodiment, the range of effective amount is 0.01 to 20 mg/kg body weight. In another embodiment, the range of effective amount is 0.1 to 25 mg/kg body weight. In another embodiment, the range of effective amount is 0.1 to 1 mg/kg body weight. In another embodiment, the range of effective amount is 0.1 to 10 mg/kg body weight. In another embodiment, the range of effective amount is 0.1 to 20 mg/kg body weight. In another embodiment, the range of effective amount is 1 to 25 mg/kg body weight. In another embodiment, the range of effective amount is 1 to 5 mg/kg body weight. In another embodiment, the range of effective amount is 1 to 10 mg/kg body weight. In another embodiment, the range of effective amount is 10 to 20 mg/kg body weight. In another embodiment, the range of effective amount is 20 to 30 mg/kg body weight. In another embodiment, the range of effective amount is 30 to 40 mg/kg body weight. In another embodiment, the range of effective amount is 40 to 50 mg/kg body weight. In another embodiment, the range of effective amount is 1 to 50 mg/kg body weight. Still other doses falling within these ranges are expected to be useful.

In another embodiment, the range of effective amount is 0.001 mg to 10 g. In another embodiment, the range of effective amount is 0.01 mg to 1 g. In another embodiment, the range of effective amount is 0.01 mg to 100 mg. In another embodiment, the range of effective amount is 0.1 mg to 100 mg. In another embodiment, the range of effective amount is 0.1 mg to 500 mg.

In another embodiment, the range of effective amount is 1 mg to 100 mg. In another embodiment, the range of effective amount is 10 mg to 500 mg. In another embodiment, the range of effective amount is 10 mg to 750 mg. In another embodiment, the range of effective amount is 0.01 mg to 100 mg. In another embodiment, the range of effective amount is 1 mg to 500 mg.

In certain embodiments, the compositions described herein is administered via intertumoral administration. In these embodiments, the effective amount of the compositions described herein may be between 0-200 nM, 0-150 nM, 0-100 nM, 0-50 nM, 0-25 nM, 25 nm-200 nM, 25-150 nM, 25-100 nM, 25-50 nM, 50 nM-200 nM, 50-150 nM, 50-100 nM, 100-200 nM, 100-150 nM, 150 nM-200 nM, about 25 nM, about 50 nM, about 100 nM, about 150 nM or about 200 nM.

In certain embodiments, the compositions described herein is administered via intraperitoneal administration. In these embodiments, the effective amount of the compositions described herein may be between 2-20 mg/kg, 0-20 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg.

In certain embodiments, the compositions described herein is administered via systemic or intravenous administration. In these embodiments, the effective amount of the compositions described herein may be between 2-10 mg/kg, 2-20 mg/kg, 0-20 mg/kg, 14-50 mg/kg, 12.5-100 mg/kg, or at least about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In certain embodiments, the compositions described herein are administered via oral administration. In these embodiments, the effective amount of the compositions described herein may be between 2-10 mg/kg, 2-20 mg/kg, 0-20 mg/kg, 0-40 mg/kg, 2-40 mg/kg, 10-40 mg/kg, 10-20 mg/kg, 20-40 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, or about 40 mg/kg.

In certain embodiments, the combination therapies above are effective to reduce the effective amount of at least one of the KRAS inhibitor, and the second therapy, such as an NFAT5 inhibitor. In certain embodiments, the effective amount of the KRAS inhibitor is reduced by 75%, 85%, 90%, 95%, or greater when compared to solo treatment. The effective amount can be reduced by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

In certain embodiments, the effective amount of the secondary therapy, such as a NFAT5 inhibitor, is reduced by 75%, 85%, 90%, 95%, or greater when compared to solo treatment. The effective amount can be reduced by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

### Kits and Articles of Manufacture

Any of the aforementioned products can be incorporated into a kit which may contain at least one of the inhibitors described herein, a pharmaceutically acceptable carrier, instructions for use, a container, a vessel for administration, or any combination thereof.

Any patent, patent application publication, or scientific publication, cited herein, is incorporated by reference herein in its entirety.

The examples are presented in order to more fully illustrate embodiments of the invention. They should in no way be construed, however, as limiting the broad scope of the invention.

### Materials and Methods

### Study Design

This experiment was performed to identify and validate NFAT5 as a therapeutic target to prevent or reduce TGFβ/EMT-driven KRAS* targeted therapy resistance in pancreatic cancer. PDAC mouse models were employed to demonstrate the sufficiency and necessity of TGFβ/EMT in regulating tumor responses to KRAS* targeting. By co-IP/MS studies, NFAT5 was identified to interact with SMAD3 and SMAD4 proteins. Subsequent spheroid assays and mouse studies revealed that the NFAT5-SMADs complex mediated TGFβ induced KRAS* targeted therapy resistance, and inhibition or depletion of NFAT5 prevented EMT and resistance. Following RNA profiling and ChIP sequencing, S100A4 was discovered as a downstream gene bound and transcriptionally activated by the NFAT5-SMADs complex. Functional assays in vitro and in vivo validated that S100A4 is essential for the TGFβ-NFAT5 axis to drive KRAS* bypass, maybe through reactivation of major KRAS*downstream pathways-MAPK and AKT. Single-cell RNA sequencing analysis revealed that tumor-associated macrophages ex-pressed high S100a4. Co-culture assays indicated that macrophages supported KRAS* bypass by providing paracrine S100A4. At least five mice were randomly allocated to different treatment groups. Both mouse survival and tumor growth were analyzed depending on downstream applications. In vitro studies were performed and repeated at least three times in two or three distinct cell lines. At least three biological replicates were used.

### PDAC Mouse Models

The iKPC and KPC PDAC mouse models were established and described previously^{7,19}. They were bred in pure C57BL/6 background. Doxycycline (dox) water (2 mg/mL, ad libitum) was administrated at 4-6 weeks of mouse age to activate transgenic KRAS^{G12D} expression in iKPC mice. The C57BL/6 mice and nude mice of both sexes were purchased from the Jackson Laboratory and employed to ensure matching sexes of the cell lines.

### Mouse Experiments

All animal experiments were approved by Rutgers' Institutional Animal Care and Use Committee (IACUC). All mice were maintained in pathogen-free conditions and received care in compliance with the regulations and certification of the Association for Assessment and Accreditation of Laboratory Animal Care International (AAALAC International).

For the induction of spontaneous tumor relapse in iKPC mice, the withdrawal of dox water was performed to cease KRAS* expression when the tumor diameter reached approximately 1 cm. Chronic pancreatitis was induced by intraperitoneal injection of caerulein starting on day 7 after KRAS* ablation, administered at a dose of 100 μg/kg, eight times a day (every hour) over two consecutive days every month. A saline solution (0.9% NaCl) was used to dissolve caerulein and served as the vehicle control.

For allograft or xenograft tumor models, human or mouse PDAC cells were transplanted orthotopically or subcutaneously into recipient C57BL/6 or nude mice at a concentration of 500,000 cells per injection, as specified in the figures. The cells were resuspended in Opti-MEM and combined with growth factor-reduced Matrigel (Corning) at a 1:1 ratio. Chronic pancreatitis induction in the transplanted tumor model involved intraperitoneal injection of caerulein starting from day 7 after tumor cell inoculation. The caerulein was administered at a dose of 100 μg/kg, eight times a day (every hour) over two consecutive days for the first week, followed by 100 g/kg, three times a day (every hour) over three consecutive days for the subsequent weeks.

For bioluminescence imaging, each mouse received a 100 L injection of D-Luciferin (15 mg/mL, i.p., Perkin Elmer). After a 10-minute interval, mice were imaged using the IVIS Spectrum Imaging System, and images were acquired and analyzed using Living Image 4.3 software.

The following reagents were employed for in vivo studies: MRTX1133 (WuXi AppTec, 10 mg/kg, i.p., BID), KRN2 (MCE, 3 mg/kg, i.p., QD), MRTX849 (MCE, 100 mg/kg, oral, QD), and α-TGFβ neutralizing antibody (BioXCell, Clone: 1D11.16.8, 250 ag per mouse, i.p., twice per week) or IgG isotype control (BioXCell, Clone: MOPC-21).

### PDAC Cell Lines and Cancer Spheroid Culture

Human cell lines MIA PaCa-2 and Panc 04.03 were purchased from the American Type Culture Collection (ATCC) and cultured in DMEM supplemented with 10% FBS and RPMI supplemented with 15% FBS and 20 Units/ml human recombinant insulin, respectively. Mouse PDAC cells were isolated from spontaneous tumors developed in iKPC or KPC mice. KPC cell lines were sustained in RPMI (Gibco) with 10% FBS (Gibco), while iKPC cell lines were cultured in RPMI with 10% Tet-approved FBS (Gibco) and doxycycline (VWR, 1 g/mL). Escaper tumor cell lines were maintained in RPMI with 10% Tet-approved FBS (Gibco). For cancer spheroid culture, cells were mixed with 50 L growth factor-reduced Matrigel (Corning) and plated in 24-well low-attachment cell culture plates (Nunc). Medium was added on top of the solidified Matrigel. We monitored both spheroid size and number for spheroid growth assays. For counting spheroid numbers, we used a cut-off of 100 m in diameter or specified, depending on the time and cell type. For measuring spheroid size, ImageJ was used. Monthly mycoplasma detection (Lonza) was performed to ensure the absence of contamination.

Reagents used for in vitro cell culture included ARS-1620 (MCE), MRTX849 (MCE), caerulein (MCE), MRTX1133 (WuXi AppTec), KRN2 (MCE), SB505124 (MCE), recombinant human and mouse TGFβ1 (Peprotech), recombinant mouse S100A4 (R&D), recombinant mouse M-CSF (Peprotech), α-TGFβ neutralizing antibody (BioXCell), α-S100A4 neutralizing antibody (R&D), and IgG isotype control (BioXCell). Dosage information is provided in the figure legends.

### Plasmid Information

All shRNAs targeting Smad2, Smad3, and Smad4 were purchased from Sigma. The sgRNAs targeting Cdkn2a, Cdkn2b, Nfat5, and S100a4 were cloned into the CRISPR/Cas9 All-in-One vector, and viruses were packaged using a second-generation lentiviral system. Puromycin (2-6 g/mL) was employed to select guide RNA- or shRNA-infected cells. For BLI imaging, PDAC cells were infected with the luciferase-mCherry reporter vector. The primer sequences for cloning are provided in Table 1.

TABLE 1

| sgRNA | | SEQ |
|---|---|---|
| cloning primers | Sequence (5′ to 3′) | ID NO |
| sgScrmb1-F | CACCGGCGAGGTATTCGGCTCCGCG | 1 |
| sgScrmb1-R | AAACCGCGGAGCCGAATACCTCGCC | 2 |
| sgCdkn2a-1F | CACCGGCAGCTCTTCTGCTCAACTA | 3 |
| sgCdkn2a-1R | AAACTAGTTGAGCAGAAGAGCTGCC | 4 |
| sgCdkn2a-2F | CACCGGCGCTGCGTCGTGCACCGGG | 5 |
| sgCdkn2a-2R | AAACCCCGGTGCACGACGCAGCGCC | 6 |
| sgCdkn2b-1F | CACCGGGATTGGGCGCCTCCCGAAG | 7 |
| sgCdkn2b-1R | AAACCTTCGGGAGGCGCCCAATCCC | 8 |
| sgCdkn2b-2F | CACCGGTCGTGCACAGGTCTGGTAA | 9 |
| sgCdkn2b-2R | AAACTTACCAGACCTGTGCACGACC | 10 |
| sgNfat5-1F | CACCGGCCGTGGGGGTAAGTAACAG | 11 |
| sgNfat5-1R | AAACCTGTTACTTACCCCCACGGCC | 12 |
| sgNfat5-2F | CACCGAAGACCAACTTCTATAACAG | 13 |
| sgNfat5-2R | AAACCTGTTATAGAAGTTGGTCTTC | 14 |
| sgNfat5-3F | CACCGAGTATCCGGTTAAAAGTGAG | 15 |
| sgNfat5-3R | AAACCTCACTTTTAACCGGATACTC | 16 |
| gS100a4-1F | CACCGGGCAGGACCCACTTACCCCC | 17 |
| gS100a4-1R | AAACGGGGGTAAGTGGGTCCTGCCC | 18 |
| gS100a4-2F | CACCGAGGTGGACACAATTACATCC | 19 |
| gS100a4-2R | AAA GGATGTAATTGTGTCCACCTC | 20 |
| gS100a4-4F | CACCGGCTCAAGGAGCTACTGACCA | 21 |
| gS100a4-4R | AAACTGGTCAGTAGCTCCTTGAGCC | 22 |
| gS100a4-5F | CACCGCATGGCAAGACCCTTGGAGG | 23 |
| gS100a4-5R | AAACCCTCCAAGGGTCTTGCCATGC | 24 |
| | | SEQ |
| rt-PCR primers | Sequence (5′ to 3′) | ID NO |
| Cdkn2a-F | GTCGCAGGTTCTTGGTCACT | 25 |
| Cdkn2a-R | CATGTTCACGAAAGCCAGAGC | 26 |
| Cdkn2b-F | CCTTTCAGGACGCGGTGTAA | 27 |
| Cdkn2b-R | AAGGTACTGACTGCACCCAC | 28 |
| Nfat5-F | TTTCCAACAGCAGCCTCCAA | 29 |
| Nfat5-R | GAGTGTAAGCTTTCCTGAGGC | 30 |
| S100a4-F | CTCAAGGAGCTACTGACCAGG | 31 |
| S100a4-R | ATTGTCCCTGTTGCTGTCCAA | 32 |
| | | SEQ |
| ChIP-qPCR primers | Sequence (5′ to 3′) | ID NO |
| S100a4-chIP-1F | CCACCCTCTCCTCTTGCATC | 33 |
| S100a4-chIP-1R | GGAAAAACCCCAGCTGCCTA | 34 |
| S100a4-chIP-2F | TCCTATTAGGCAGCTGGGGT | 35 |
| S100a4-chIP-2R | CATTCTTCTCCCTCCCAGCG | 36 |
| S100a4-chIP-3F | TTGAGGGGTAGGCTTTGCAG | 37 |
| S100a4-chIP-3R | CCCTGGGGAGATCAGAGGAA | 38 |

### Luciferase Reporter Assay

Mouse S100a4 promoter was cloned into pGL4.12[iluc2CP] vector (Promega). Renilla luciferase vector (Promega) was used as the internal control. pGL4.12 vector and Renilla luciferase vector were co-transfected into iKPC PDAC cells using Lipofectamine 2000 (Invitrogen). After 24 hours, the medium was replaced, and cells were subjected to various treatments for an additional 24 hours. Subsequently, cells were collected for luciferase reporter assay using the Dual-Luciferase Reporter Assay Kit (Promega) according to the manufacturer's instructions. The firefly luciferase signal was normalized to Renilla luciferase prior to comparison among different treatment groups.

RNA Extraction, qRT-PCR, mRNA Sequencing, and GSEA

RNA extraction from 2-D or 3-D cultured cell samples was conducted using the RNA Extraction Kit from Qiagen. Matrigel-based 3-D cells were isolated using Cell Recovery Solution from Corning. RNA concentration was assessed using NanoDrop 2000. The RNA samples were either sent for RNA-seq analysis to the Genomic Center at Rutgers or Genewiz (Azenta), or reverse transcribed for qRT-PCR analysis. The preparation of cDNA utilized 5× All-In-One RT MasterMix from Applied Biological Materials, and the PCR reactions were prepared with SYBR Green PCR Master Mix from Applied Biosystems or Bio-Rad. qRT-PCR was executed on the CFX Opus 96 (Bio-Rad), with statistical analysis conducted using GraphPad Prism. For mRNA sequencing, the parameters were NGS-75 nt Paired End, utilizing the Illumina Next Generation Sequencing instrument.

In the case of scRNA-seq, tumors were dissociated into single cells using the Tumor Dissociation Kit from Miltenyi Biotec, followed by the removal of dead cells using the Dead Cell Removal Kit (Miltenyi Biotec). Cells were resuspended in PBS+0.05% BSA at a concentration of 500-600 cells/L for library preparation (10× Genomics). At least 100,000 cells were recovered per sample, ensuring a minimum of 20,000 reads per cell for NGS sequencing. NGS sequencing and bioinformatic analysis were conducted at the Genomic Center at Rutgers New Jersey Medical School. Raw reads were first subjected to barcode deconvolution and aligned to the mm10 reference genome using cellranger (v7.1.0). Subsequent data processing was conducted with the Seurat package (v4.3) in R. Cells deemed of low quality—defined by a percentage of reads of mitochondrial origin exceeding 10%, a percentage of reads of ribosomal origin surpassing 45%, less than 1000 feature counts, or exceeding 7000 feature counts-were filtered from the dataset. Correction for ambient nucleotides was executed utilizing SoupX (v1.6.2), and read counts underwent normalization employing the scTransform method as previously described (45). Sample integration was accomplished using the Seurat integrate function (46), and subsequent clustering via UMAP was performed based on nearest neighbors, using 40 principal components.

GSEA was performed using GSEA software (4.3.2). The qRT-PCR primer sequences are listed in Table 1.

### Antibodies, Western Blot, IP, Co-IP/MS, IHC, and Trichrome Stain

Antibody information can be found in Table 2. Western blot analysis, IP, co-IP, and IHC staining were conducted following standard protocols. Human TMA slides were purchased from Biomax. Cell fractionation and Masson's Trichrome Staining were carried out using commercial kits, adhering to manufacturers' protocols. MS analysis of proteins pulled down by endogenous SMAD2, SMAD3, SMAD4, NFAT5, and IgG was performed by the Proteomics Core Facility at Rutgers New Jersey Medical School. Specifically, IP samples were subjected to SDS-PAGE separation. Each sample's gel lane was excised for in-gel trypsin digestion, and the resulting peptides were analyzed using LC-MS/MS on an Orbitrap Fusion Lumos Tribrid mass spectrometer coupled with the Ultimate 3000 nano-LC system (Thermo Scientific). MS/MS spectra were searched against the UniProt mouse database (55,336 sequences, downloaded 8/30/2021) using the Sequest search engine through the Proteome Discoverer platform (version 2.4) with a false discovery rate less than 1% for both proteins and peptides. Protein abundance ratios were determined using the Label-Free Quantitation (LFQ) method.

Human tumor tissues were evaluated using two criteria: the percentage of stained area (0% for no staining, 1-10% scored as 1, 11-50% as 2, 51-80% as 3, and 81-100% as 4) and the intensity of staining in the nuclei or cytoplasm (no staining as 0, weak staining as 1, moderate staining as 2, and strong staining as 3). The overall scores were determined by multiplying the assigned scores for the percentage of stained area and staining intensity.

TABLE 2

| Antibody | Application | Vendor | Cat. # |
|---|---|---|---|
| ACTIN | WB | Sigma | A2228 |
| VINCULIN | WB | Sigma | V4505 |
| Anti-mouse IgG, | WB | CST | 7076 |
| HRP-linked | | | |
| Anti-rabbit IgG, | WB | CST | 7074 |
| HRP-linked | | | |
| Anti-rabbit IgG, | WB | CST | 5127 |
| conformation | | | |
| specific HRP-linked | | | |
| ARG1 | IHC | CST | 93668 |
| ERK | WB | CST | 4695 |
| pERK | IHC, WB | CST | 4370 |
| F4/80 | IHC | CST | 70076 |
| Ki67 | IHC | Thermo | RM-9106-S1 |
| pS6 (S235/236) | IHC, WB | CST | 4858 |
| pS6 (S240/244) | WB | CST | 5364 |
| SMAD2 | WB, ChIP, co-IP | CST | 3103 |
| pSMAD3 (S423/425) | WB | Abcam | ab52903 |
| SMAD3 | WB, ChIP, co-IP | CST | 9523 |
| SMAD4 | WB, ChIP, co-IP | CST | 38454 |
| S100A4 | WB, IHC | Proteintech | 16105-1-AP |
| NFAT5 | WB, IHC, ChIP, | Novus | NB120-3446 |
| | co-IP | | |
| TGFB1 | IHC | Proteintech | 21898-1-AP |
| SNAI1 | WB | CST | 3879 |
| SNAI2 | WB | CST | 9585 |
| ZEB1 | WB | CST | 70512 |
| total RAS (H + K) | WB | Abcam | ab191595 |
| Cyclophilin A | WB | CST | 2175 |
| Lamin A/C | WB | CST | 4777 |

### Isolation and Culture of Bone Marrow-Derived Macrophages

Mouse bone marrow-derived macrophages were isolated as previously described¹⁰. Immature macrophages (M0) were induced by recombinant mouse M-CSF (20 ng/mL, BioLegend) for 7 days. Murine IFNγ (10 ng/mL, Peprotech) and LPS (100 ng/mL, Peprotech) were employed for M1 polarization, while murine IL-4 (20 ng/mL, Peprotech) was utilized for M2 polarization. Tumor conditional media were generated by adding fresh complete cell culture medium when tumor cells reached 70-80% confluence. After 24 hours, the medium was collected, filtered through a 0.45 m filter, and stored at −80° C. The working solution consisted of a 1:1 ratio of the collected medium mixed with fresh medium.

### ChIP-seq and ChIP-qPCR

ChIP was conducted following the protocol of the SimpleChIP® Plus Enzymatic Chromatin IP Kit (Cell Signaling Tech., Inc, #9005). Briefly, cells were cross-linked with 1% paraformaldehyde and then quenched with 0.125 mol/L glycine. Subsequently, cells were lysed on ice for 30 minutes using lysis buffer. Chromatin DNA was fragmented to around 200-500 bp through Micrococcal Nuclease digestion, followed by sonication using a sonicator with a 102C probe (Branson Sonifier 450) for 5 cycles of 20 seconds on and 20 seconds off at an output of 15%. The lysate was then incubated overnight with anti-SMAD2, SMAD3, SMAD4, NFAT5, or IgG antibodies at 4° C. Immune complexes were washed and separated using ChIP-Grade Protein G Magnetic Beads. Chromatin was eluted from the antibody/protein G magnetic beads at 65° C. for 30 minutes with gentle vortexing (1,200 rpm). DNA was subsequently reverse-crosslinked at 65° C. for 4 hours, followed by DNA purification through column elution. The purified DNA samples were sequenced at Azenta Life Sci using the Illumina NovaSeq™ 6000 Sequencing System (2×150 bp). For ChIP-qPCR validation, DNA samples were prepared as described above, and primers were designed based on S100a4-binding peaks from the ChIP-seq data, detailed in Table 1.

### Statistical Analysis

Statistical analysis was conducted using the unpaired two-tailed Student t test or one-way ANOVA to generate P values. Kaplan-Meier survival curves were generated using GraphPad Prism, and the Log-rank (Mantel-Cox) test was employed for statistical analysis in survival analysis. The P values: ns, not significant; *, P<0.05; **, P<0.01, ***, P<0.001; ****, P<0.0001.

### Example I: Chronic Pancreatitis-Induced Resistance to Krasi Hinges on Tgfβ Signaling

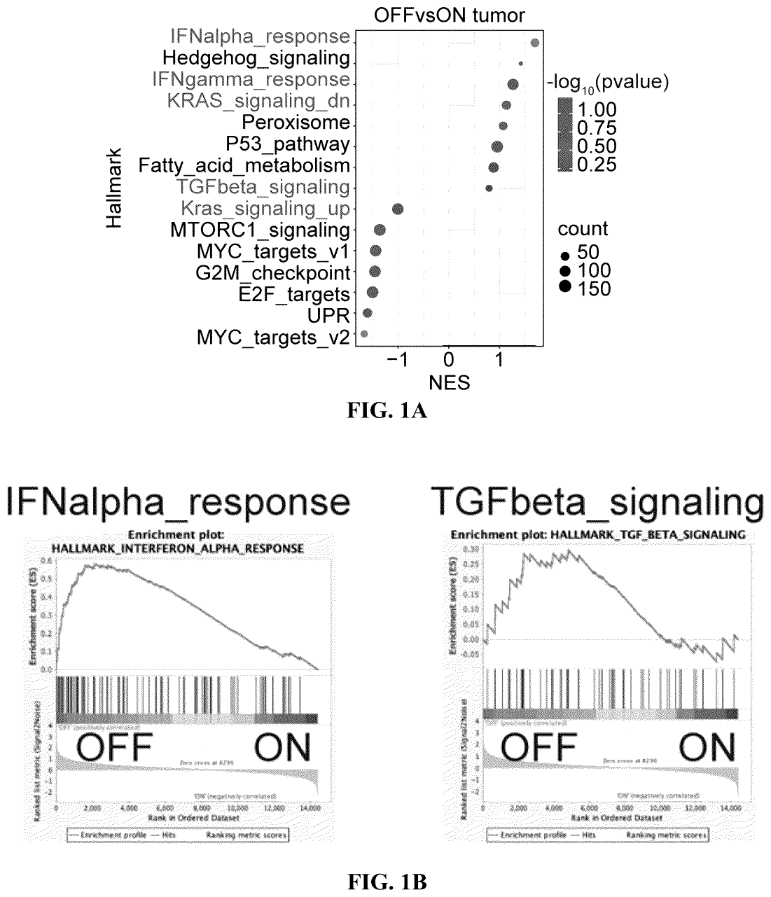

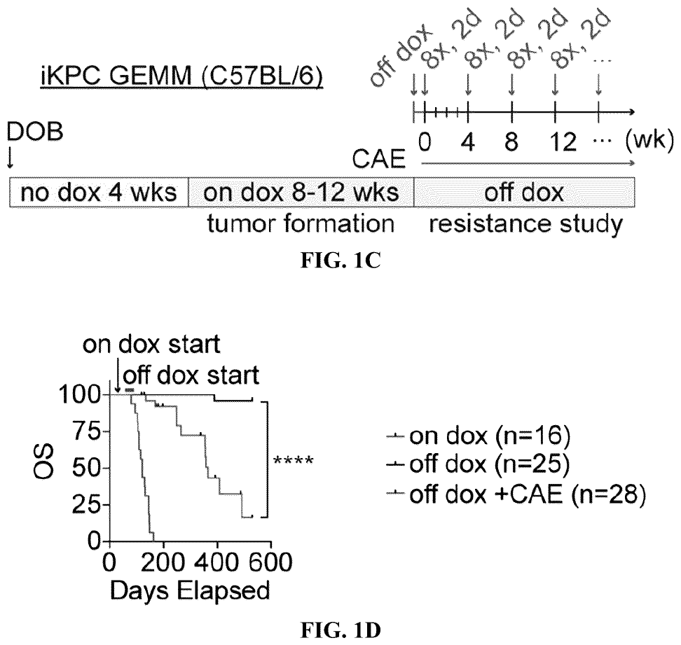

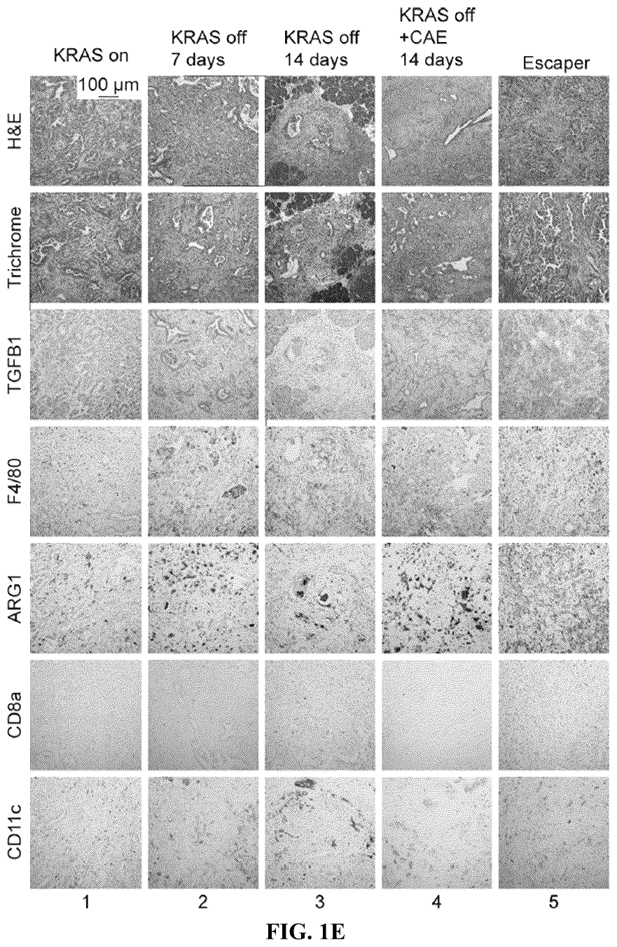

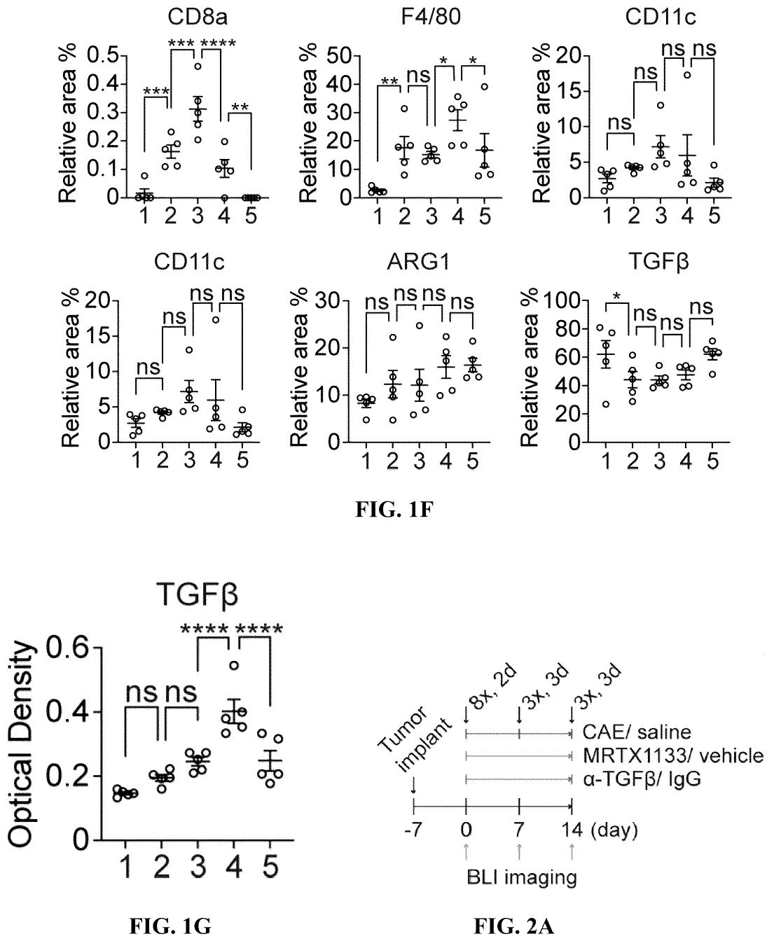

Pancreatitis is a key risk factor for PDAC (Gandhi et al., 2022; Kirkegard et al., 2018), resulting in TGFβ elevation in the microenvironment (Glaubitz et al., 2023; Ishihara et al., 1998). This prompted us to investigate whether chronic pancreatitis fosters the bypass of KRAS* dependency in PDAC. iKPC (p48-Cre, tetO_LKras^{G12D} ROSA_rtTA, Trp53^{Lox/+}) genetically engineered mice on a C57BL/6 back-ground, in which Kras^{G12D} expression was regulated by the tet-ON promoter and induced by doxycycline (dox) treatment were utilized (Ying et al., 2012). RNA profiling data comparing KRAS*-expressing (KRAS* on) and KRAS*-depleted (KRAS* off) tumors from iKPC mice revealed an elevation of inflammatory pathways and TGFβ signaling in KRAS* off tumors versus KRAS* on tumors (FIGS. 1A and 1B), indicating that these factors play a crucial role in KRAS* therapy resistance.

To investigate the role of pancreatitis in regulating KRAS* bypass, a well-established method for inducing acute or chronic pancreatitis through repetitive injections of caerulein (CAE) was utilized (Ferreira et al., 2017; Komar et al., 2017). These mice were administered doxy water starting at 4 weeks of age to initiate tumorigenesis. Upon reaching a pancreatic tumor size of approximately 1 cm in diameter, dox was discontinued to halt KRAS* expression. One week after dox withdrawal, mice received injections of either vehicle or caerulein (CAE) to induce chronic pancreatitis^{17,18} (FIG. 1C). While control mice remained tumor-free, at least 36% (10 escapers out of 28 confirmed, 2 undetermined) of the CAE-treated mice developed escaper tumors less than or around a year after treatment (FIG. 1D). These escaper tumors were undifferentiated, expressed high levels of TGFβ, and contained a significant number of M2-like macrophages as reflected by robust F4/80 and ARG1 expression (FIGS. 1E and 1F).

Using histological analysis, increased fibrosis in residual tumor lesions was observed when compared with KRAS*-expressing tumors at the onset of treatment regimens (FIG. 1E). These lesions continued to decrease in size by day 14 after dox withdrawal. In contrast, the CAE-treated group displayed pancreatitis characterized by pancreatic damage with increased fibrosis, elevated TGFB1 expression, and enhanced infiltration of F4/80+macro-phages by day 14 (FIGS. 1E and 1F). Although an increase in the number of TGFβ+ cells was not observed, pancreatitis induction significantly enhanced the TGFβ signal intensity, as reflected by the optical density analysis of IHC staining (FIG. 1G). Moreover, despite low infiltration levels, a significant increase in CD8+ T cells was observed following KRAS* depletion (FIGS. 1E and 1F). However, the induction of pancreatitis reduced CD8+ T cell infiltration, and escaper tumors were found to be deprived of CD8+ T cells, similar to KRAS*-expressing primary tumors. In contrast, significant changes in immune-suppressive myeloid cells and dendritic cells were not observed, as indicated by ARG1 and CD11c staining, respectively (FIGS. 1E and 1F).

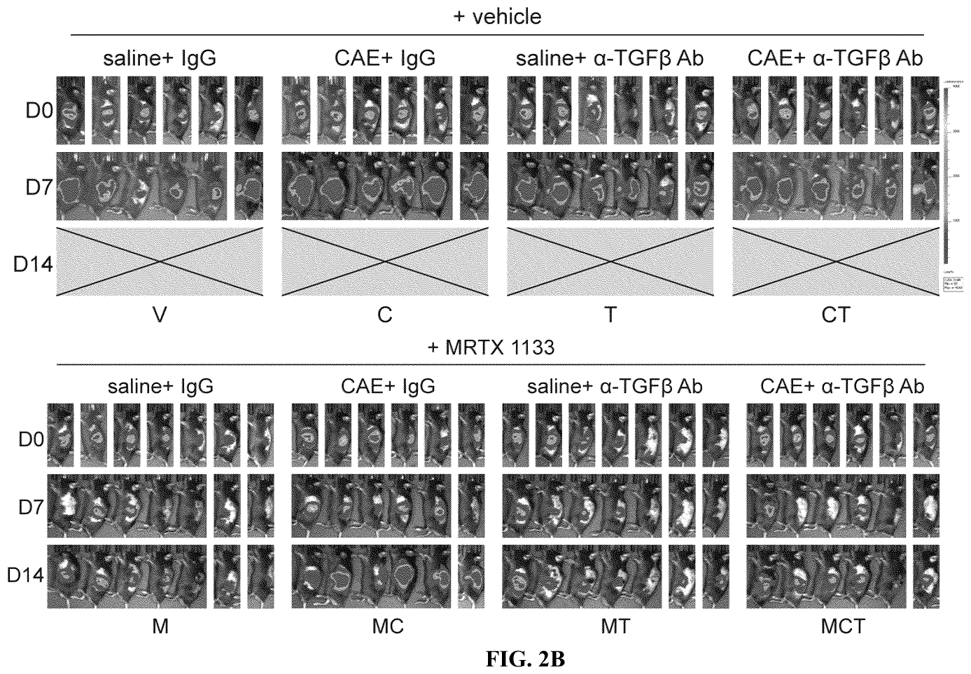

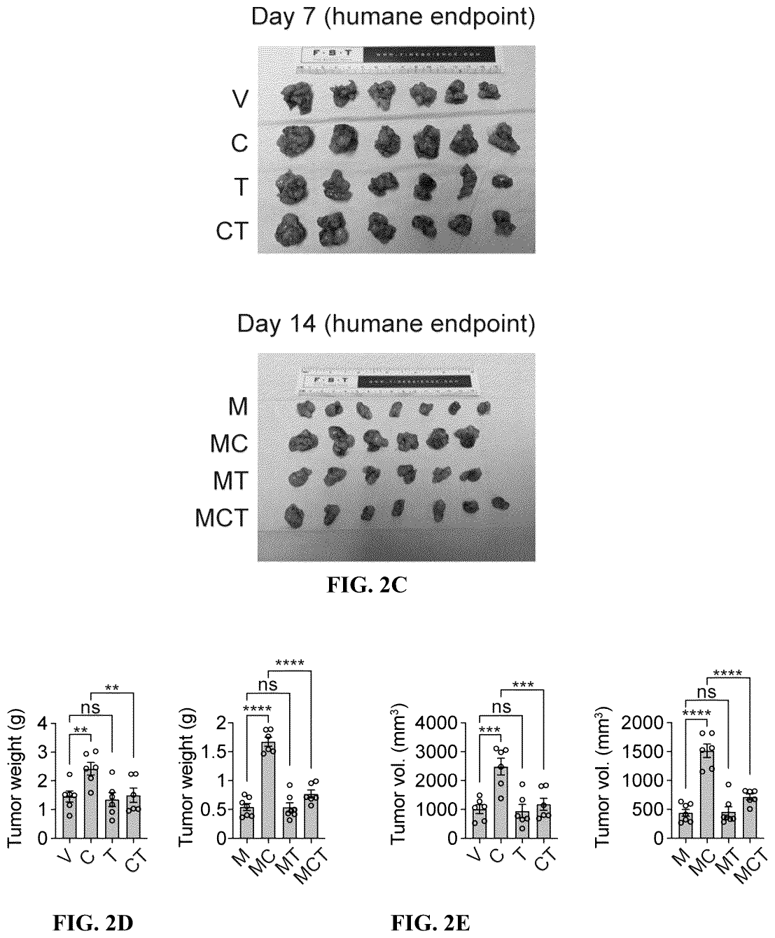

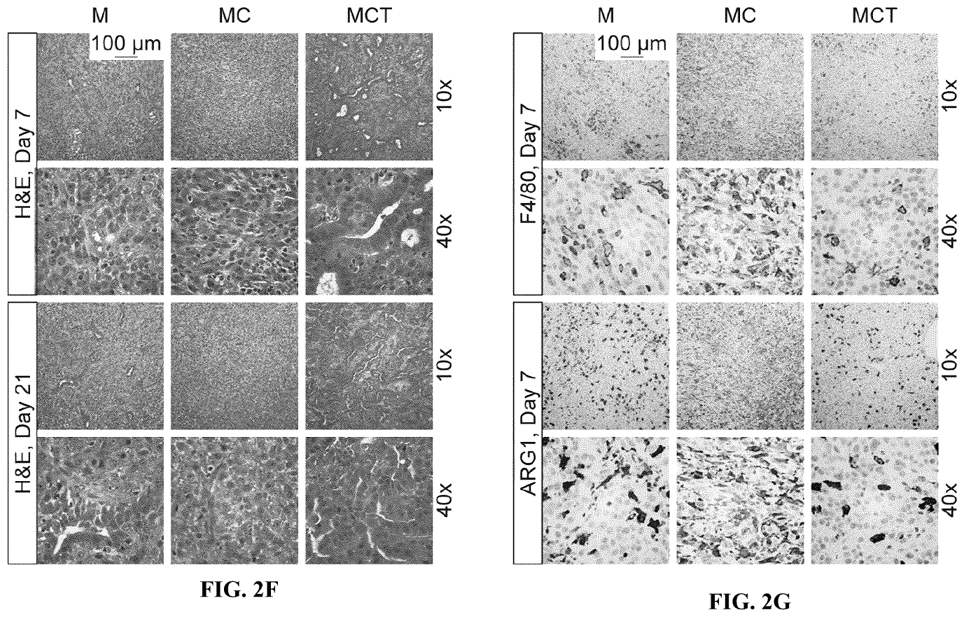

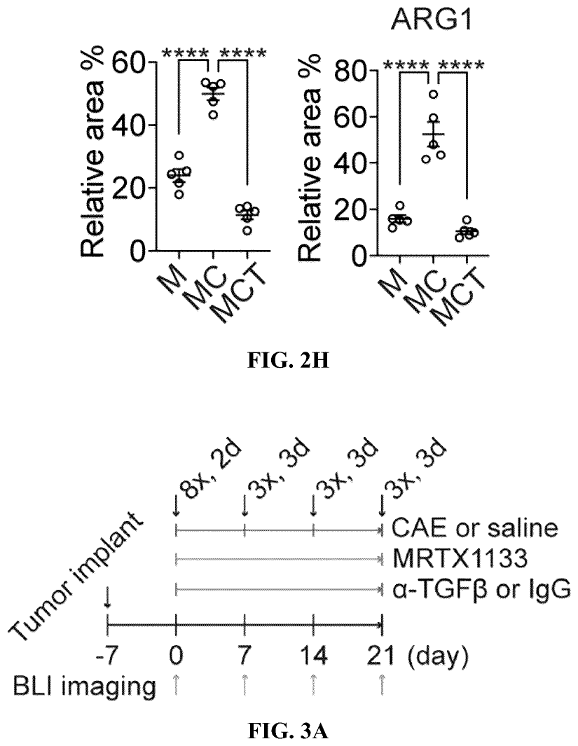

To further corroborate these findings in an alternative genetic model, parallel pharmacological studies were conducted using orthotopically transplanted luciferase-expressing KPC (p48-Cre, lox-stop-lox Kras^{G12D}, Trp53^{lox/+})¹⁹ PDAC cells in C57BL/6 mice. One week post-implantation, tumor-bearing mice received treatment with the KRAS^{G12D} inhibitor (G12Di) MRTX1133, CAE, and/or an anti-TGFβ neutralizing antibody (FIG. 2A). The data reveals that inducing pancreatitis with CAE promotes KPC PDAC tumor growth compared with the vehicle control while neutralizing TGFβ suppresses this effect (FIG. 2B-2E). Although G12Di significantly reduces tumor growth, pancreatitis induction leads to resistance. However, the acceleration of tumor growth by CAE is mitigated by TGFβ blockade (FIG. 2B-2E). Notably, TGFβ neutralization alone has minimal impact on tumor growth, and no additive or synergistic anti-tumor effects between KRAS inhibition and TGFβ neutralization was observed in this KPC tumor model.

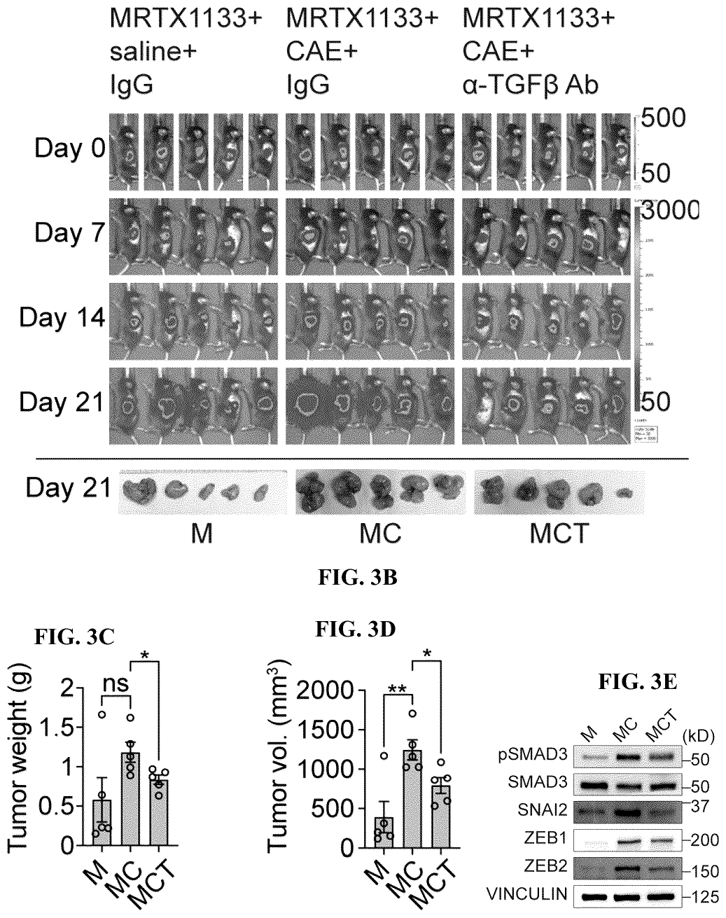

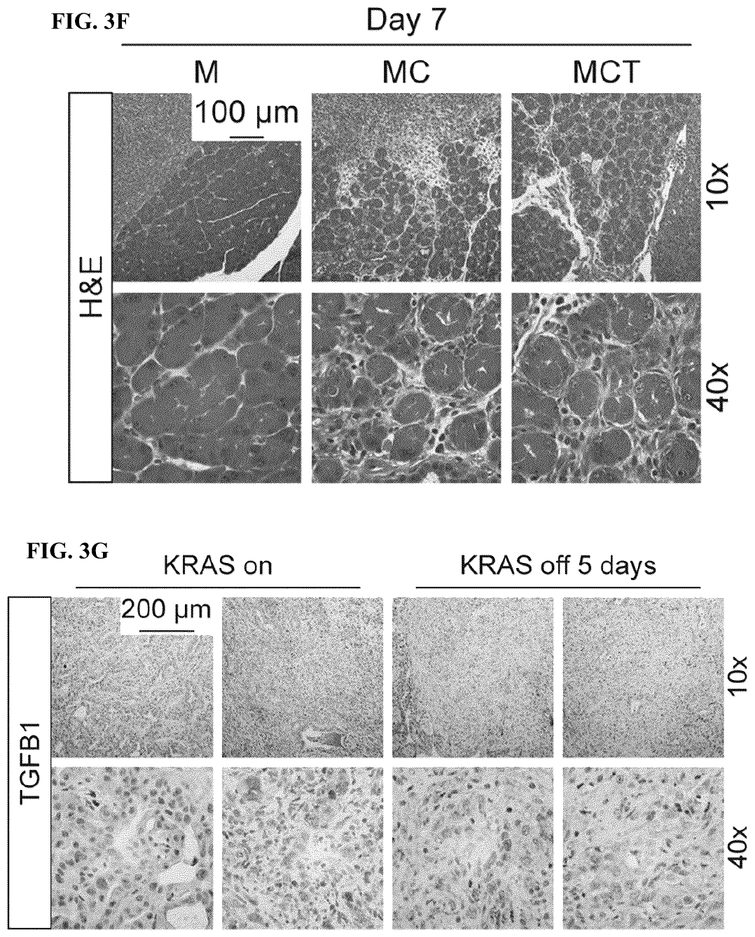

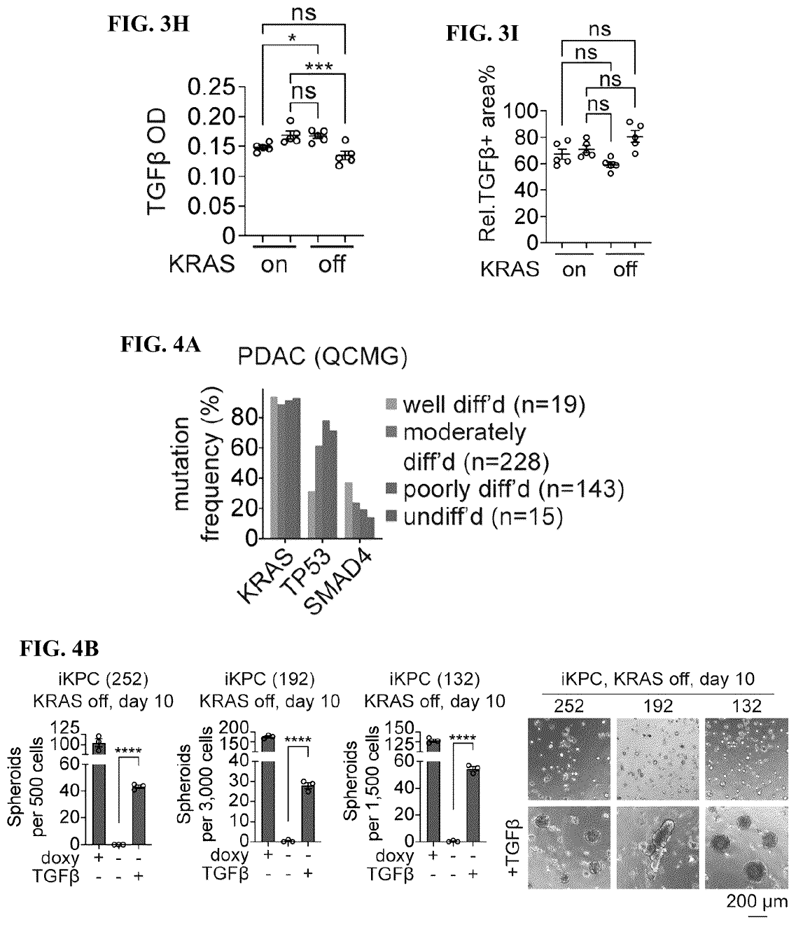

In another independent study, similar results were observed showing that chronic pancreatitis induction in the CAE;G12Di-treated group was associated with significantly faster tumor progression compared with vehicle, G12Di-treated controls. This CAE-driven tumor progression was partially attenuated with TGFβ blockade (FIG. 3A-3D). Western blot analysis of collected tumors revealed that CAE treatment induced SMAD3 phosphorylation and the upregulation of EMT transcription factors (TFs) such as SNAI2, ZEB1, and ZEB2 (FIG. 3E), indicating TGFβ signaling activation. TGFβ blockade downregulated these markers (FIG. 3E). In addition, by histological examination on days 7 and 21, the data showed that CAE treatment induced inflammation in the normal pancreas, with minimal impact on immune cell infiltration upon TGFβ blockage (FIG. 3F). CAE;G12Di-treated tumors lost the ductal-like phenotype and were more undifferentiated relative to vehicle;G12Di controls (FIG. 2F). TGFβ blockage restored a differentiated ductal-like phenotype and reduced the number of M2-like tumor-associated macro-phages (TAMs) in the CAE;G12Di-treated tumors (FIG. 2F-2H). In summary, this data indicates that pancreatitis induction accelerates pancreatic cancer growth and contributes to KRAS* targeting resistance in a TGFβ-dependent manner.

### Example 2: Tgfb Drives Kras* Bypass Via Nfat5

The TGFβ signaling pathway exhibits multifunctionality²⁰, capable of activating target genes through both SMADs-dependent (canonical) and -independent (non-canonical) mechanisms²¹. R-SMADs, such as SMAD2 and SMAD3, undergo phosphorylation by TGFβ receptors (TGFβR) upon ligand binding, forming heterotrimers with SMAD4 to regulate gene expression. The non-canonical TGFβ pathway involves the activation of other signaling pathways, including MAPK, JNK/p38 μMAPK, PI3K/Akt cascades, and Rho-like GTPases²².

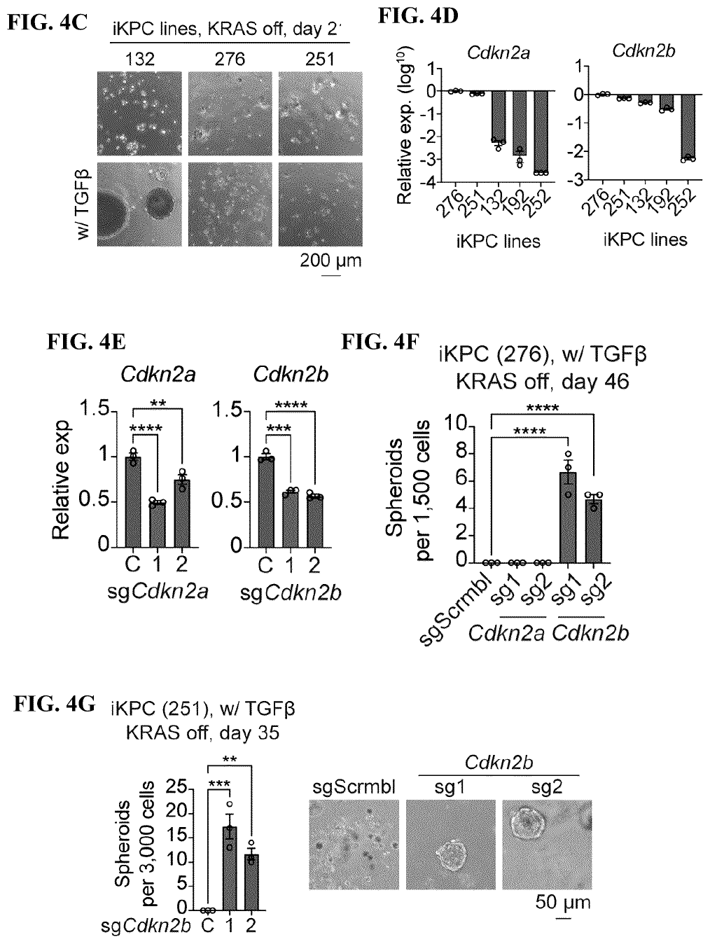

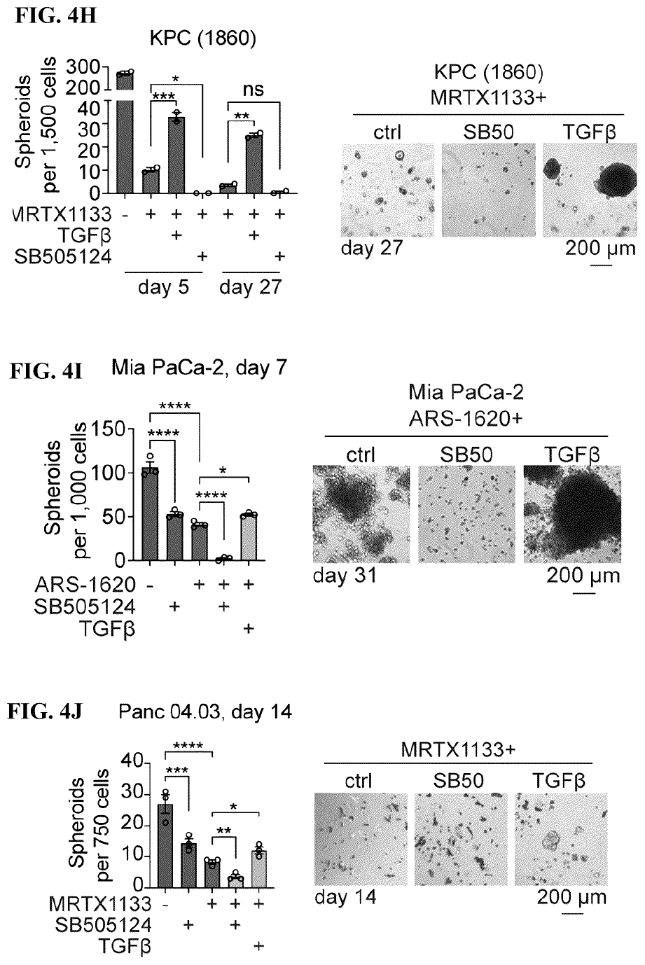

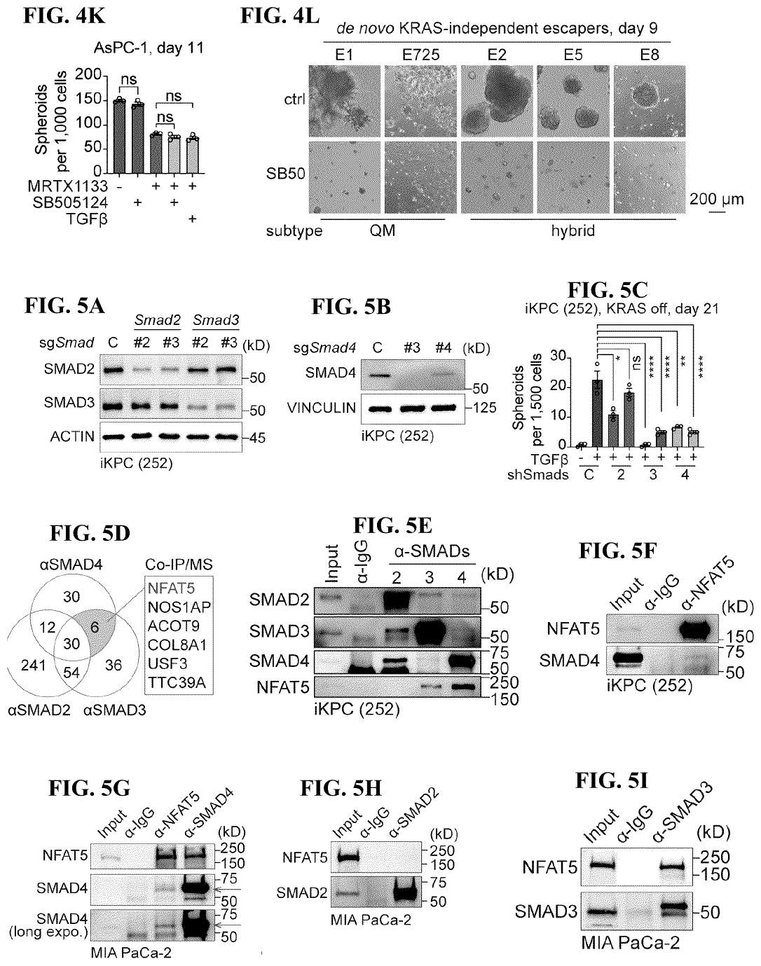

Despite the genetic inactivation of SMAD4 in approximately one-third of PDAC cases, analysis of the TCGA PDAC dataset indicates a decreased frequency of genetic alterations of SMAD4 in poorly differentiated and undifferentiated tumor subtypes (FIG. 4A), indicating that an active canonical TGFβ pathway is present in these EMT-associated aggressive PDAC subtypes.

TGFβ serves as a major inducer of EMT, a process often positively associated with resistance to targeted, chemo-, and immunotherapies in various cancers²³⁻²⁵. TGFβ is highly expressed in PDAC tissues, regardless of KRAS targeting (FIG. 1E, 3G-3I). In line with our previous findings¹⁰, we observed that TGFβ1 efficiently promoted several iKPC PDAC cell lines to bypass KRAS* in spheroid assays (FIG. 4B). Notably, some iKPC cell lines (251, 276) were unable to bypass KRAS* under TGFβ1 treatment (FIG. 4C). Gene expression analysis revealed higher expression of tumor suppressors cyclin-dependent kinase inhibitor 2a and 2b (Cdkn2a and Cdkn2b) in iKPC cell lines that failed to bypass KRAS* compared to those that could (FIG. 4D). Consistent with previous reports indicating that TGFβ can stimulate CDKN2B expression via the SMAD4-SMAD2/3-FOXO complex to induce cell cycle arrest^{26,27}, knockdown of Cdkn2b, rather than Cdkn2a, prevented TGFβ1-induced cell death and enabled PDAC cells to become KRAS*-independent (FIG. 4E-4G). Thus, we conclude that CDKN2B serves as a key barrier to TGFβ-driven KRAS* bypass.

To investigate the impact of TGFβ on the development of resistance to KRASi, we utilized a KPC PDAC cell line and two human KRAS* PDAC cell lines for spheroid formation analysis. TGFβ1 actively promoted the formation of cancer spheroids resistant to KRASi from both mouse and human PDAC cells with the intact TGFβ pathway (KPC [1860], Mia PaCa-2, and Panc 04.03, respectively, FIG. 4H-4J). Conversely, the combined inhibition of the TGFβ receptor (TGFβRi) with KRASi led to the eradication of cancer spheroids (FIG. 4H-4J). However, SMAD4-deficient human PDAC cells AsPC-1 did not exhibit a response to TGFβ1 or TGFβRi (FIG. 4K), indicating the necessity of canonical TGFβ pathway activation for KRASi resistance. Furthermore, de novo generated KRAS*-independent escaper tumors from iKPC mice, which exhibit a hybrid or QM-like phenotype²⁸, displayed hypersensitivity to TGFβRi (FIG. 4L). These findings illustrate the essential role of TGFβ pathway activation for the survival of KRASi-resistant cells.

NFAT5 Interacts with SMAD3 and SMAD4

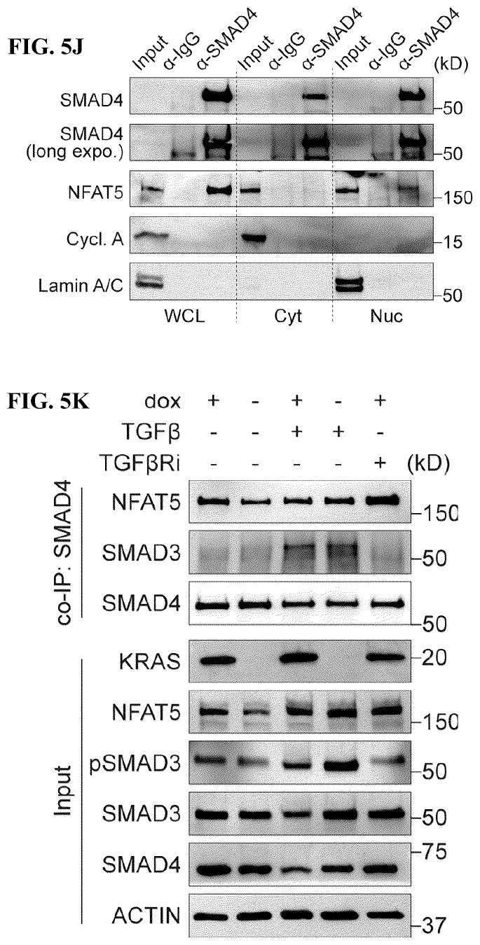

To determine whether the canonical or non-canonical TGF3 pathway is crucial for the development of KRAS* independency, we conducted knockdown experiments targeting SMADs to block the canonical pathway. (FIG. 5A-5B) The results indicated that canonical TGFβ pathway factors, Smad4 and Smad3, but not Smad2, were essential for KRAS* bypass in PDAC (FIG. 5C). Notably, SMAD3 and SMAD4, along with their downstream EMT transcription factors (TFs), have been considered chemically undruggable. Attempts to target upstream elements of the TGFβ signaling cascade, such as TGFβRi, have led to cardiovascular toxicities and chronic inflammation²⁹. To address this challenge, specific interacting factor(s) of SMAD3 and SMAD4 essential for KRAS* bypass were targeted to enhance KRASi efficacy while minimizing adverse effects.

SMAD3 and SMAD4 exhibit weak DNA binding affinity through the MH1 domain¹⁵. Instead, other TFs or transcriptional regulators cooperatively bind with them or act as pioneer factors, facilitating chromatin opening and enabling SMADs to access their binding sites¹⁵. This distinctive feature prompted us to explore chemically druggable SMAD3 and SMAD4 interactors that mediate TGFβ-driven KRAS* bypass. Through optimized co-immunoprecipitation (co-IP)/mass spectrometry (MS) analysis using endogenous SMAD proteins as baits, we successfully identified several TFs or transcriptional regulators. Among them, nuclear factor NFAT5 emerged as the sole interactor that was bound with both SMAD3 and SMAD4, excluding IgG and SMAD2 (FIG. 5D). We validated protein interactions of endogenous SMAD3, SMAD4, and NFAT5 through co-IP/western blot, demonstrating the conservation of complex formation in both human and mouse PDAC cells (FIG. 5E-5I). Moreover, this protein interaction was observed exclusively in the nucleus, not in the cytoplasm (FIG. 5J), indicating that DNA is involved in the formation of the NFAT5-SMADs complex.

To demonstrate whether the formation of NFAT5-SMADs depends on KRAS* and TGFβ signaling pathways, co-IP and western blot analysis was performed to examine the interaction be-tween NFAT5 and SMAD4 in iKPC cells following modulation of KRAS* or TGFβ signaling. The input control data indicate that both KRAS* and TGFβ signaling pathways upregulate NFAT5 expression, with KRAS* having a dominant regulatory effect compared with TGFβ (FIG. 5K). The co-IP data reveal that SMAD4 and NFAT5 interact constitutively, regardless of KRAS* or TGFβ signaling activation (FIG. 5K). However, the interaction between SMAD3 and SMAD4 is TGFβ-dependent, and SMAD3 is required for TGFβ-driven KRAS* bypass (FIGS. 5C and 5K). In KRAS*-depleted cells, the enhanced interaction between NFAT5 and SMAD4 induced by TGFβ may be attributed to increased NFAT5 expression.

Therefore, the formation of the NFAT5-SMADs complex is TGFβ-dependent, supporting the biochemical changes observed with CAE treatment.

### NFAT5 is Upregulated in PDAC

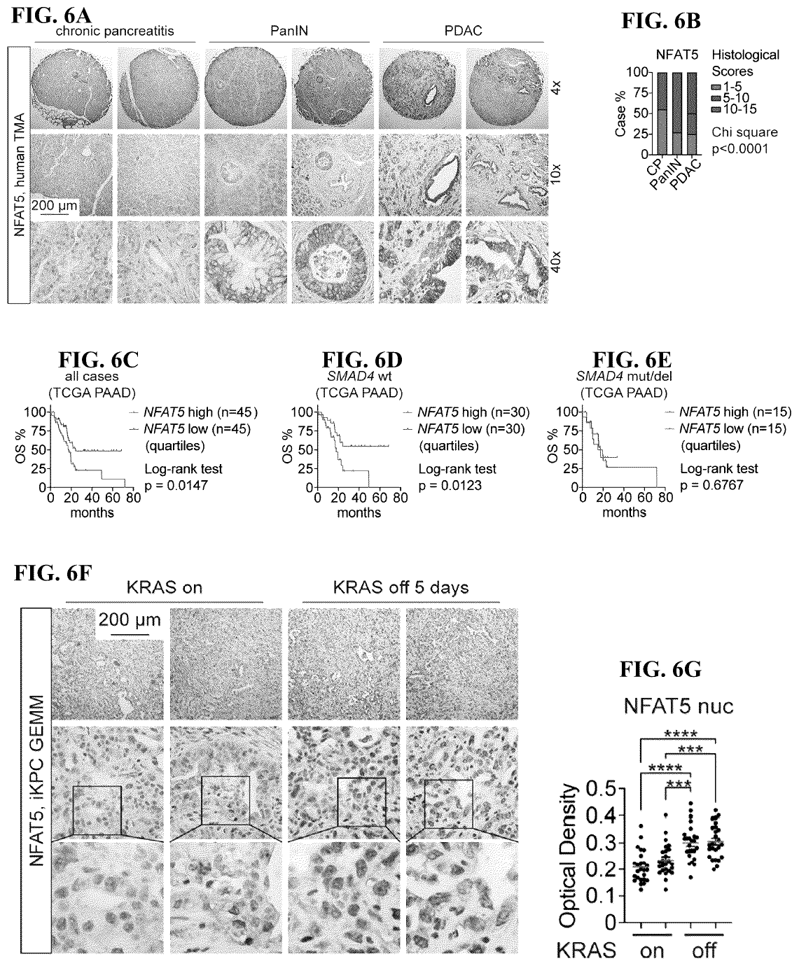

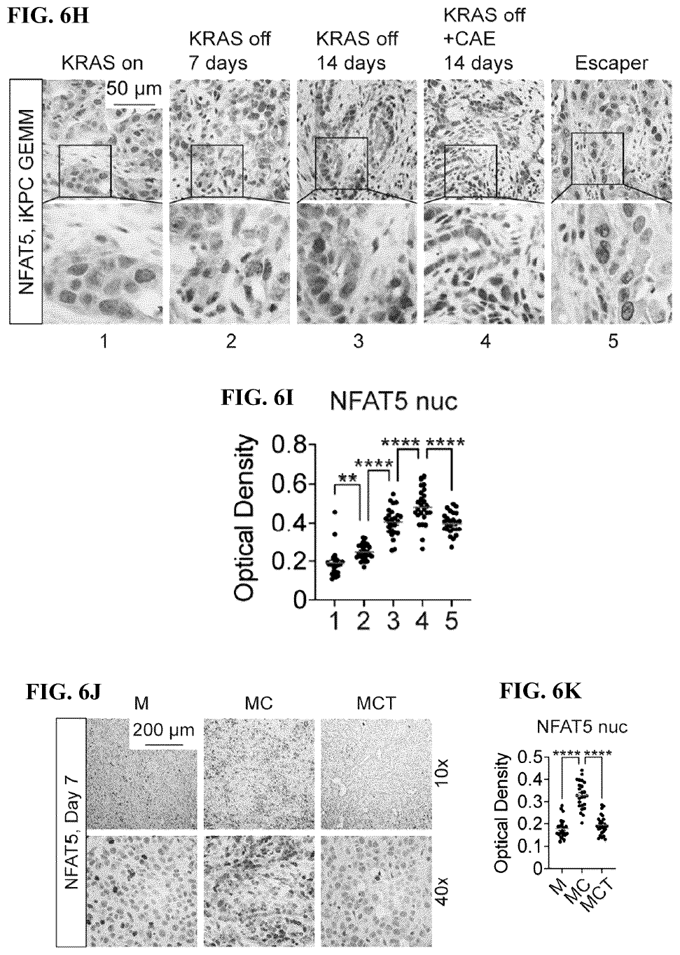

In contrast to other members of the Rel family, NFAT5 is insensitive to calcium/calcineurin signaling, typically exists in a dimerized state, and does not synergize with FOS or JUN³⁰⁻³². NFAT5 plays a regulatory role in ambient hypertonicity³³ and is involved in the development and activation of immune cells¹⁶. A tissue microarray (TMA) study revealed a positive correlation between NFAT5 expression and pancreatic tumorigenesis (FIG. 6A-6B). Correspondingly, elevated NFAT5 expression is associated with poor overall survival in the TCGA PDAC dataset (FIG. 6C). Patient stratification indicates that patients with high NFAT5 expression have significantly shorter overall survival compared with those with low NFAT5 expression in the SMAD4 wildtype (SMAD4 wt) cohort (FIG. 5D). However, in patients with SMAD4 mutations or deletions (SMAD4 mut/del), overall survival is similar regardless of NFAT5 expression levels (FIG. 6E). These findings indicate that NFAT5 plays a key role in patients with intact SMAD4 by interacting with the canonical TGFβ pathway.

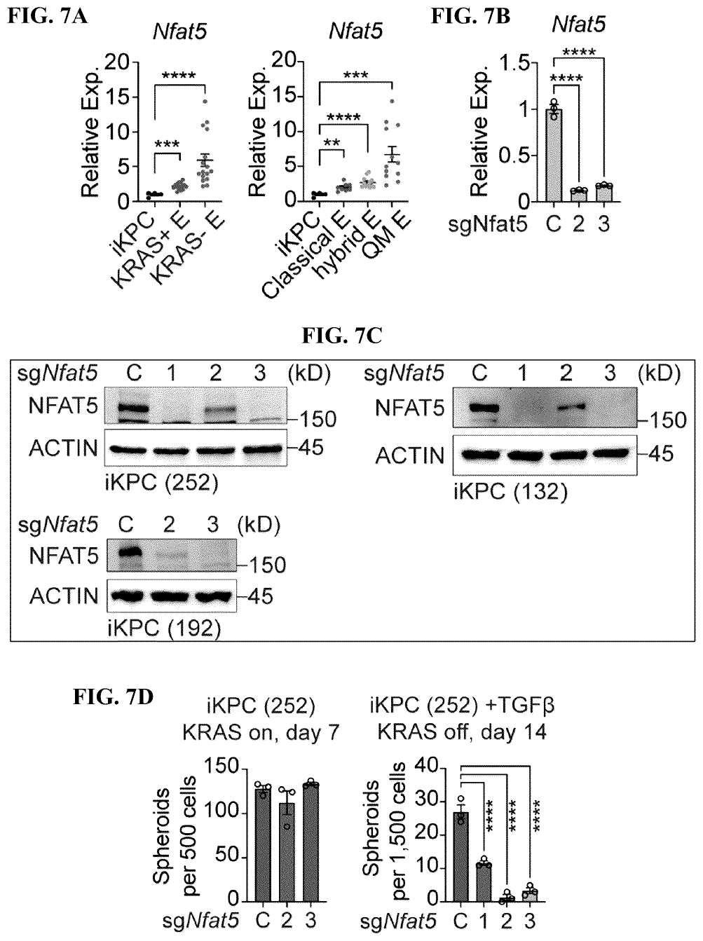

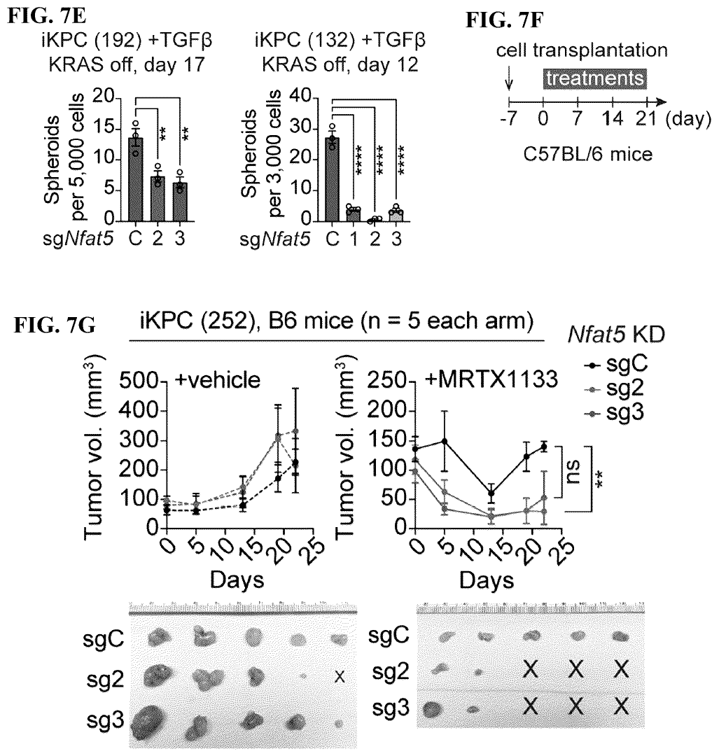

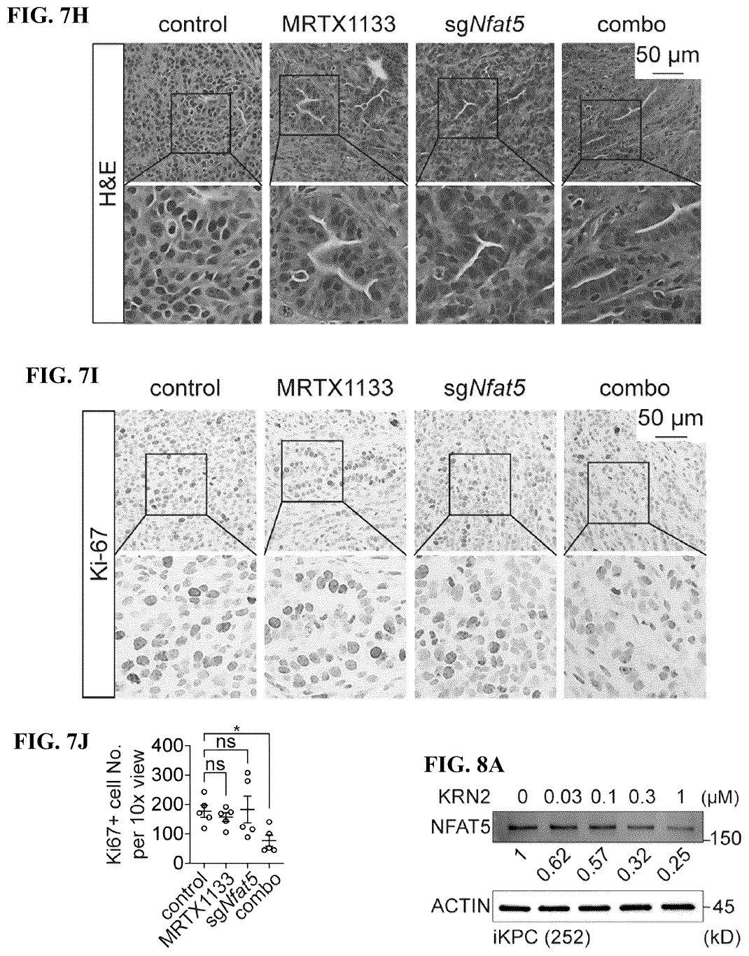

Histological analysis of mouse tumors revealed a significant increase of nuclear NFAT5 following KRAS* depletion (FIGS. 6F and 6G), particularly after chronic pancreatitis induction and in escaper tumors(FIG. 6H-6I). Neutralization of TGFβ su-pressed nuclear NFAT5 expression (FIG. 6J-6K), indicating that NFAT5 has potential as a downstream target of TGFβ. Additionally, de novo-generated iKPC escaper tumors, especially KRAS*-independent ones, exhibited a significant upregulation of Nfat5 expression compared with primary KRAS*-expressing tumors (FIG. 7A). Subtype analysis indicated that Nfat5 expression is highest in QM-like escapers, followed by hybrid ones (FIG. 7A). Collectively, these data point to Nfat5 as a potential regulator of EMT-associated KRAS* bypass.

### NFAT5 is Essential for TGFβ Driven KRAS* Targeting Resistance

To elucidate the role of NFAT5 in regulating tumor responses to KRAS* targeted therapy, we conducted Nfat5 knockdown in iKPC PDAC cells (FIG. 7B-7C). While proving dispensable for the growth of KRAS*-expressing cancer spheroids, Nfat5 emerged as an essential factor for TGFβ-driven, KRAS*-independent spheroid formation (FIG. 7D-7E). Consistently, the knockdown of Nfat5 attenuated tumor growth in vivo under treatment with G12Di, in contrast to the vehicle control (FIG. 7F-7G). Compared to untreated tumors, Nfat5 knockdown induced tumor differentiation and a decrease in Ki67-positive cancer cells (FIG. 7H-7J), signifying the suppression of EMT and cancer cell proliferation.

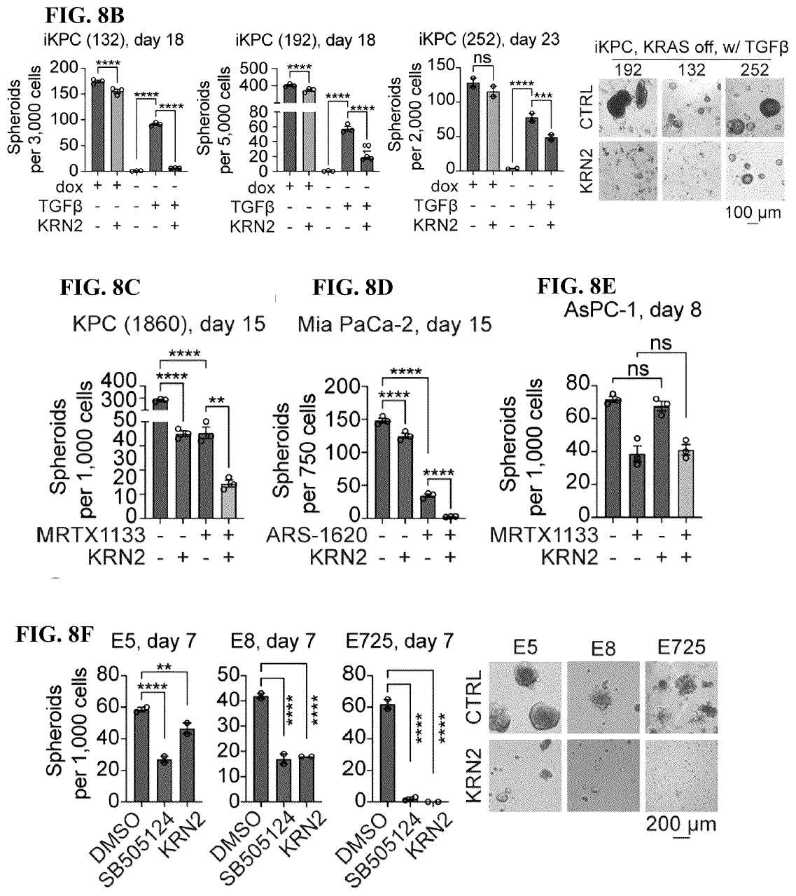

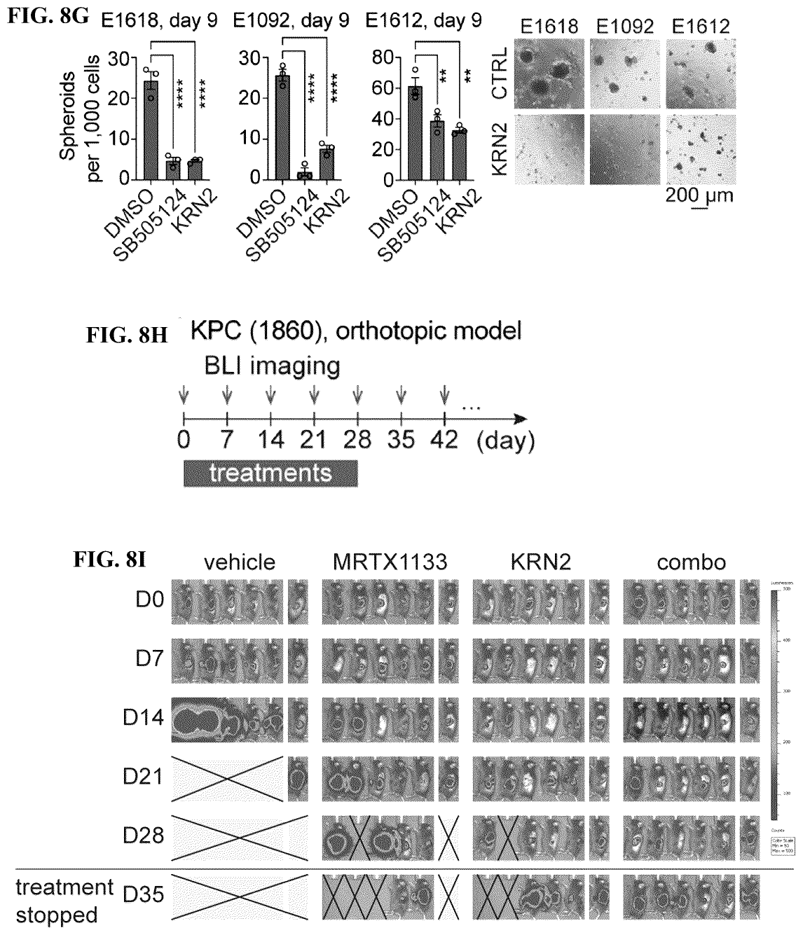

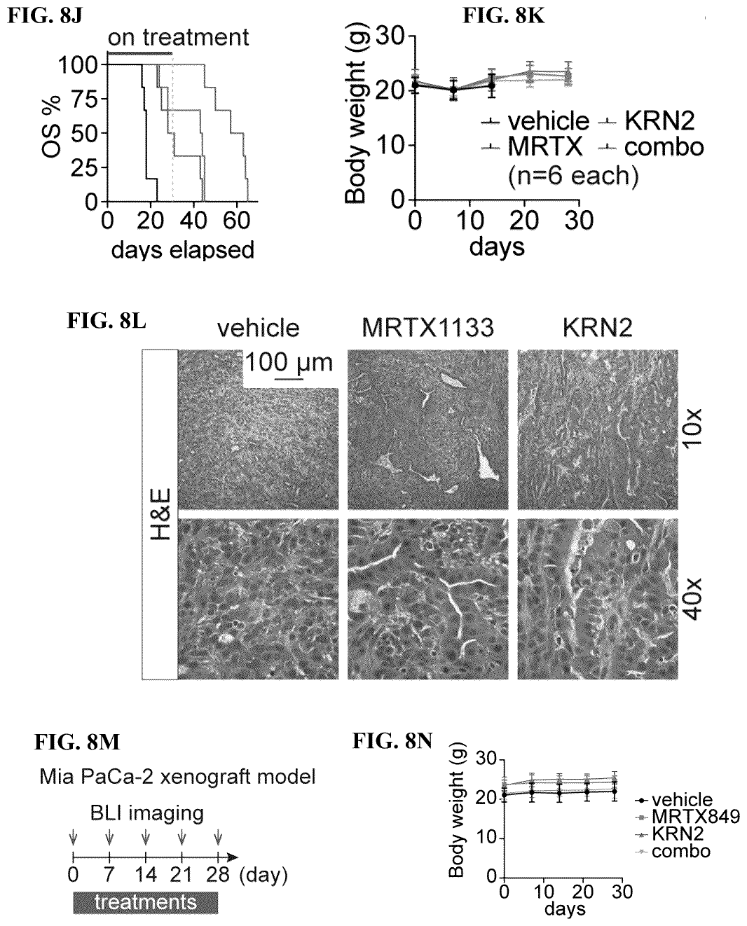

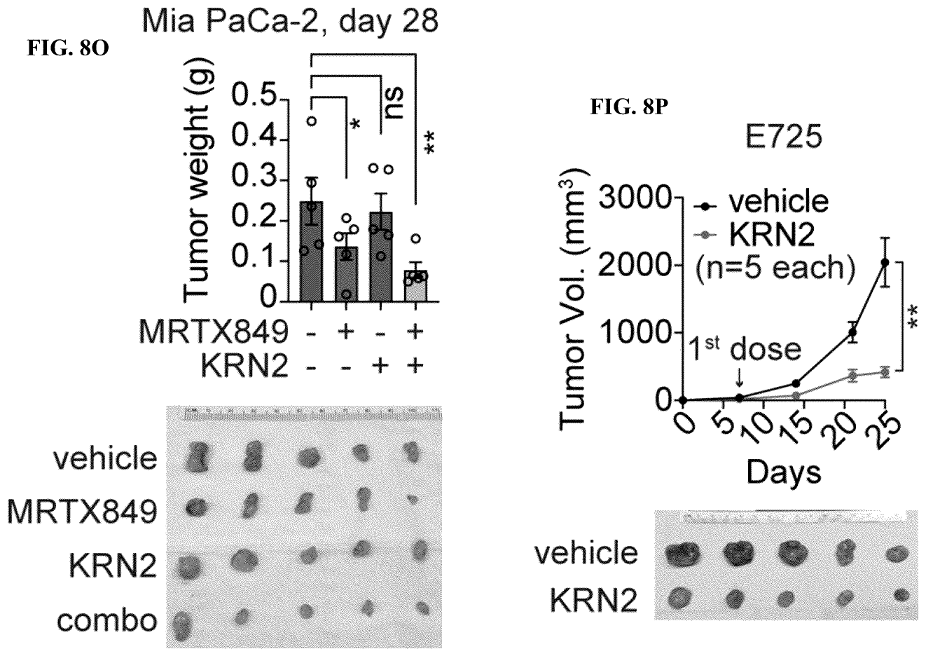

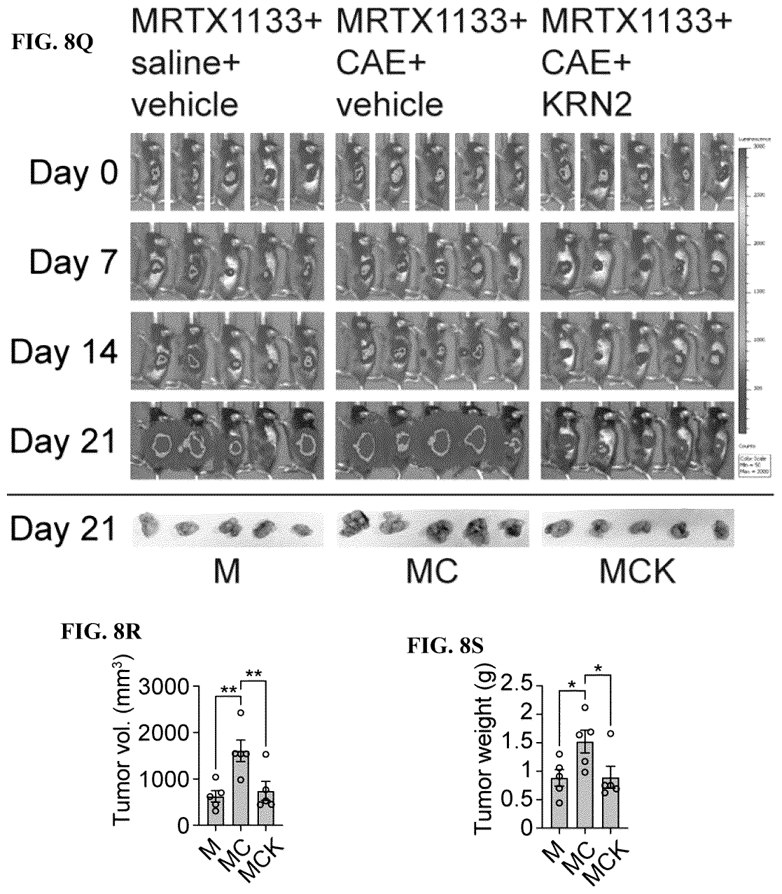

NFAT5 can be inhibited by a small molecule compound, KRN2, which disrupts the binding of NF-κB p65 to the NFAT5 promoter region³⁴. We confirmed that KRN2 suppressed Nfat5 expression in PDAC cells at concentrations higher than 0.3 μM (FIG. 8A). Consistent with genetic results, KRN2 demonstrated the following inhibitory effects: (1) impairment of TGFβ-driven KRAS* bypass (FIG. 8B), (2) attenuation of KRASi persistent cancer spheroid formation, excluding SMAD4-deficient cancer spheroid formation (FIG. 8C-8E), (3) suppression of QM-like or hybrid KRAS*-independent escaper spheroid growth (FIG. 8F-8G), (4) inhibition of mouse allograft tumor growth in combination with KRASi (FIG. 8H-8L), (5) reduction of human xenograft tumor growth cooperatively with KRASi (FIG. 8M-80), (6) restriction of escaper tumor growth in vivo (FIG. 8P), and (7) blockage of chronic pancreatitis-induced KRASi resistance (FIG. 8Q-8S). KRN2 exhibited minimal systemic toxicities, reflected by stable mouse weights during treatment in both allograft and xenograft models (FIGS. 8K and 8N). Additionally, KRN2 induced tumor differentiation compared to the vehicle control in the KPC transplanted model (FIG. 8L). In contrast to Nfat5 knockdown (FIG. 7G), KRN2 monotherapy prolonged mouse survival in immune-competent mice (FIG. 8J), indicating that NFAT5 has non-cancer cell autonomous functions in regulating tumor maintenance. Thus, NFAT5 forms a transcriptional regulatory complex with SMAD3 and SMAD4 to mediate TGFβ-driven KRAS* bypass and sustain EMT-associated escaper tumor growth.

### Example 3: Nfat5-Smads Complex Transcriptionally Activates S100A4

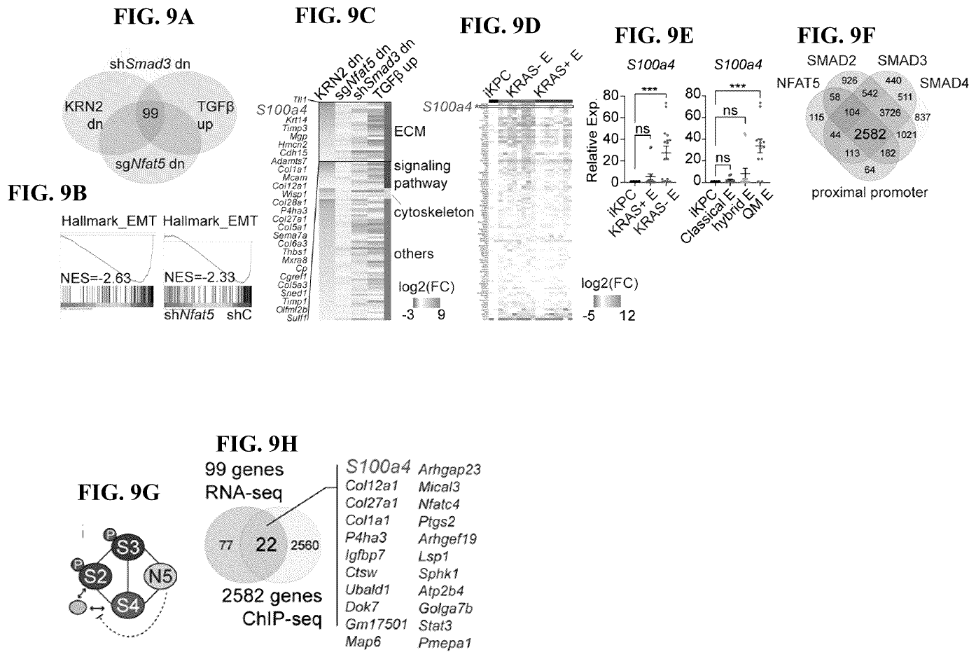

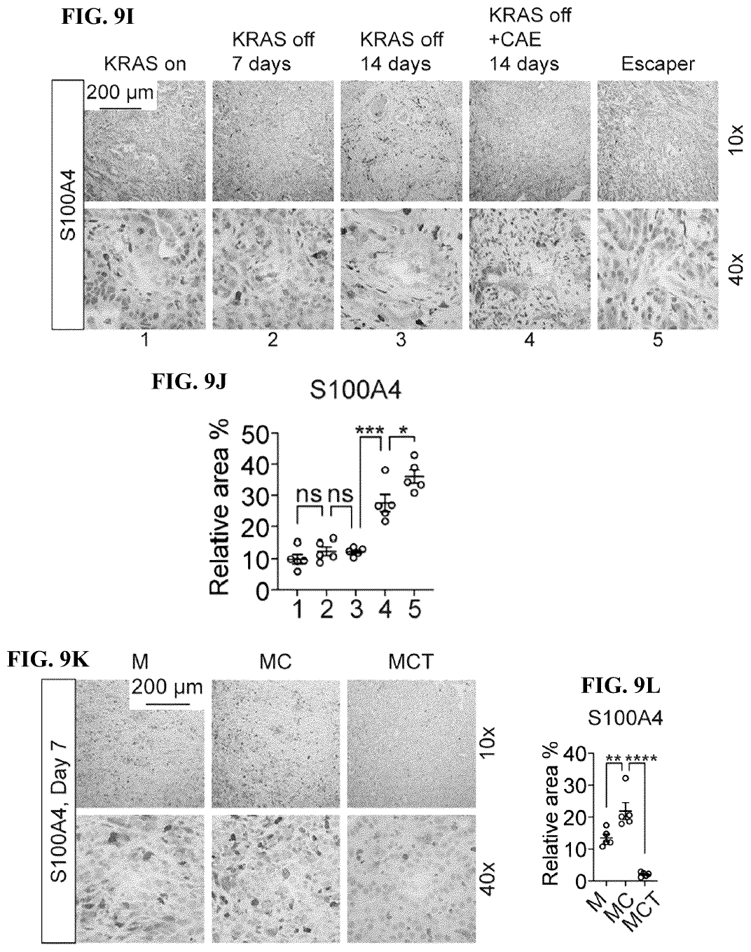

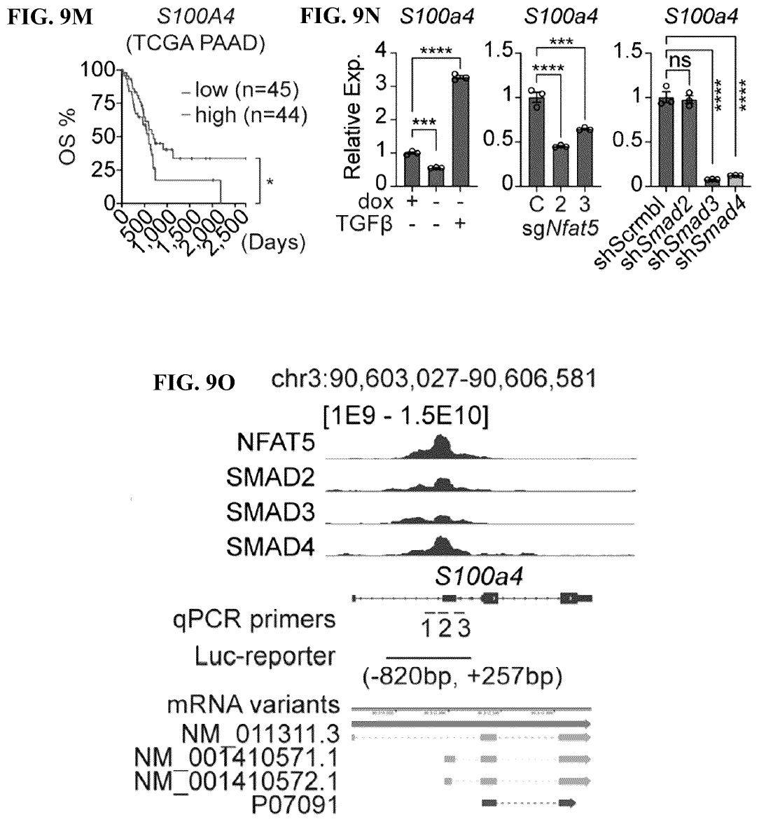

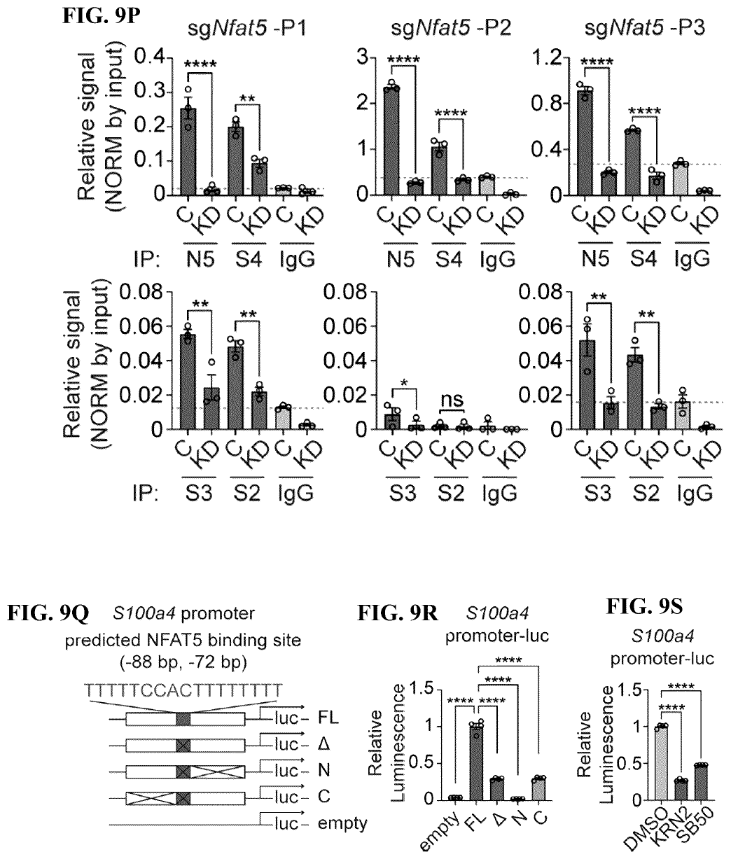

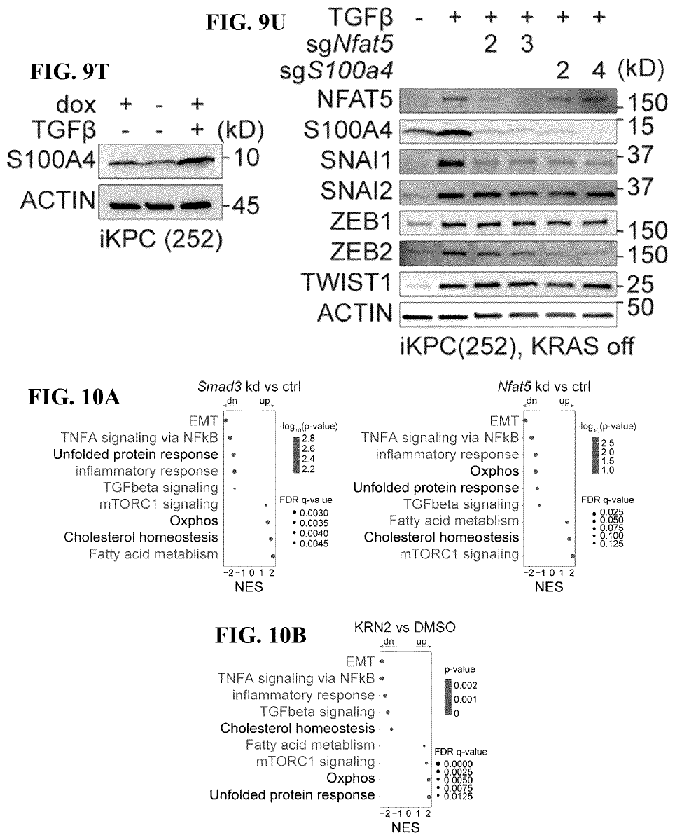

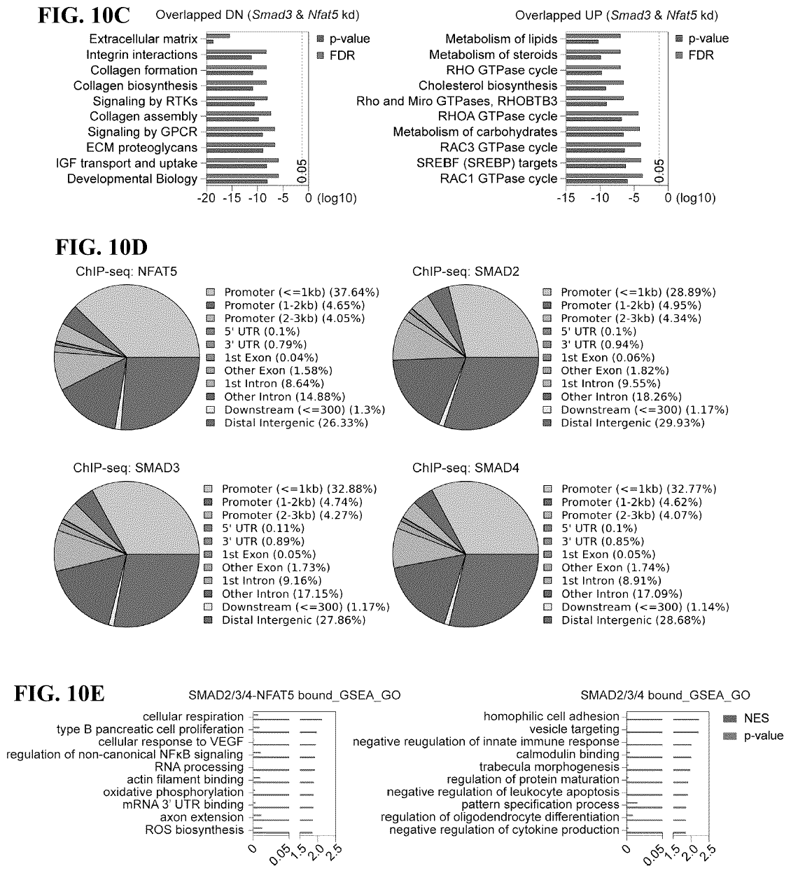

To unravel the regulatory role of the NFAT5-SMADs complex in KRAS* bypass, we conducted transcriptomic analysis in TGFβ-treated, KRAS*-depleted iKPC spheroids (FIG. 9A). Notably, the knockdown of Nfat5 and Smad3, as well as the inhibition of NFAT5 by KRN2, resulted in the top downregulation of gene sets related to EMT and NFκB, while upregulating genes associated with fatty acid metabolism and mTORC1 signaling (FIG. 9A). The inhibition of NFAT5 by KRN2 similarly suppressed EMT and NFxB gene signatures (FIG. 9B and FIG. 10A-10B). Specifically, the overlapping genes downregulated by Nfat5 and Smad3 knockdown were enriched in extracellular matrix (ECM) and collagen biosynthesis pathways, while upregulated genes were enriched mainly in GTPase regulation and lipid metabolism (FIG. 10C).

Further analysis, intersecting the three datasets with TGFβ-upregulated genes in KRAS^{G12D}-depleted iKPC cells, revealed 99 overlapping genes, with approximately one-third of them associated with the ECM (FIGS. 9A and 9C). S100 calcium-binding protein A4 (S100a4), a key regulator of ECM and EMT³⁵, emerged as one of the top genes upregulated by TGFβ but downregulated following interference with Nfat5 and Smad3 (FIG. 9C). Comparative analysis with other candidate genes highlighted elevated expression of S100a4 in KRAS*-independent escaper tumors, particularly in the QM subtype (FIG. 9D-9E).

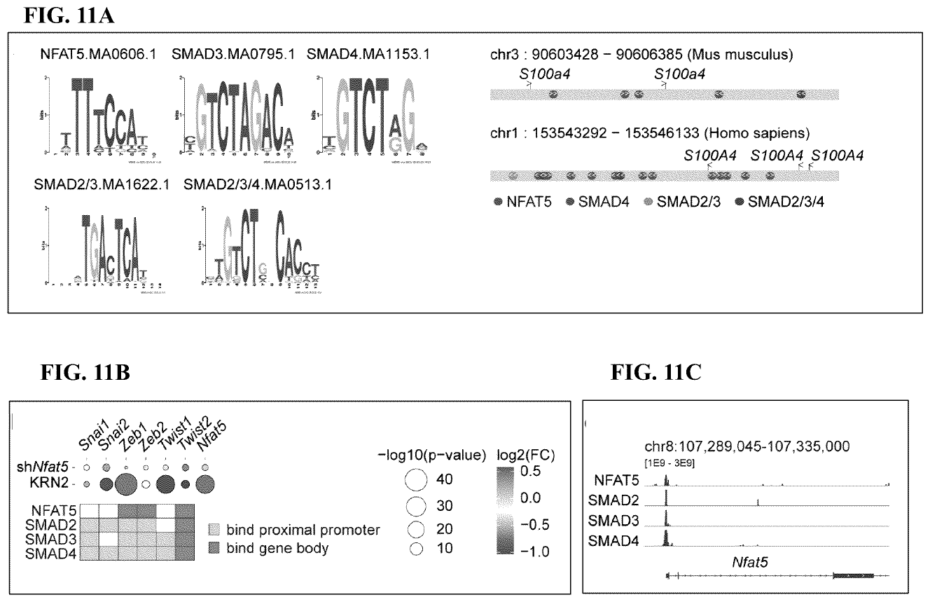

To identify DNA bound by the NFAT5-SMADs complex, we conducted Chromatin IP followed by next-generation sequencing (ChIP-seq) using antibodies binding to NFAT5, SMAD2, SMAD3, and SMAD4 (FIG. 9F, FIG. 10D). Analysis revealed that 2582 genes were bound by NFAT5 and SMAD2/3/4 at the proximal promoter (±1 kb, p<0.01), while only 113 genes were bound by NFAT5 and SMAD3/4 (FIG. 9F). Additionally, 3726 genes were still bound by the SMAD2/3/4 complex. This data indicates that NFAT5 does not interfere with the formation of heterotrimers of SMADs. Instead, the NFAT5-containing complex blocks the interaction between SMAD2 and its partners, regulating a unique group of genes (FIG. 9G). Accordingly, genes bound by the SMAD2/3/4-NFAT5 complex were enriched in oxidative phosphorylation and non-canonical NF-κB signaling, while genes bound by the SMAD2/3/4 complex were enriched in cell adhesion and development (FIG. 10E). Overlapping the 2582 genes bound by the NFAT5-SMADs complex with the 99 candidate genes upregulated by the NFAT5-SMADs complex identified 22 genes, many of which are ECM-related genes, including S100a4 (FIG. 9H). Additionally, we predicted TF binding motifs using TFmotifView³⁶ and identified several NFAT5 and SMADs binding sites in the human S100A4 and mouse S100a4 gene loci (FIG. 11A).

S100A4 belongs to the S100 protein family and is localized in the cytoplasm, nuclei, and the ECM³⁵. Upon calcium binding, it undergoes a conformational change to recognize its target proteins. S100A4 plays a multifaceted role in cancers, particularly in PDAC, where it is reported to regulate tumor growth, metastasis, and angiogenesis via activating Src and focal adhesion kinase signaling pathways³⁷. Our observations indicate that S100A4 is expressed in both cancer cells and stromal cells in PDAC, with elevated expression noted after pancreatitis induction and in escaper tumors compared to primary tumors (FIG. 9I-9J). The neutralization of TGFβ suppressed pancreatitis-induced S100A4 expression in the TME (FIG. 9K-9L), indicating the TGFβ-NFAT5 axis as the major upstream regulator of S100a4. Moreover, S100A4 emerges as a potential prognostic marker for human PDAC³⁸, with high expression positively correlated with poor patient survival (FIG. 9M).

NFAT5 was previously identified as a downstream effector of integrin a604 signaling, activating S100A4 expression in breast and colon cancer cells³⁹-4⁰. We revealed that S100a4 was upregulated by TGFβ in KRAS*-depleted iKPC spheroids, a process dependent on NFAT5 and SMAD3/4 but not SMAD2 (FIG. 9N). Interestingly, KRAS* signaling also upregulated S100a4 expression (FIG. 9N), which relates to its function in driving EMT. ChIP-seq data revealed enriched binding peaks of NFAT5 and SMADs around the second exon of S100a4 (FIG. 9O), where the transcription start site (TSS) of mRNA variants NM_001410571 and NM_001410572 is located. Through ChIP-qPCR analysis comparing Nfat5 knockdown to wildtype control, we demonstrated that the binding of the NFAT5-SMADs complex to the promoter region of S100a4 depended on NFAT5 (FIG. 9P). The signal intensity of NFAT5 and SMAD4 was notably higher than that of SMAD3 and SMAD2, indicating that the complex binds DNA via NFAT5 and SMAD4.

Additionally, we utilized a luciferase reporter under the control of the S100a4 promoter to demonstrate that TGFβ activated S100a expression in KRAS*-depleted iKPC PDAC cells, while depletion of the predicted NFAT5 binding site in the S100a4 promoter completely diminished the activity of the luciferase reporter (FIG. 9Q-9R). Furthermore, chemical inhibition of NFAT5 or TGFβR suppressed S100a4 activation (FIG. 9S). We also validated the upregulation of S100A4 by TGFβ at the protein level in cancer spheroids (FIG. 9T). Together, these data support the role of NFAT5 as a pioneer factor in priming SMADs to bind to the S100a4 promoter and activate its expression.

The canonical TGFβ signaling pathway activates EMT TFs at both transcriptional and post-translational levels⁴¹. Despite not observing consistent transcriptional regulation of EMT TFs by Nfat5 or direct binding of NFAT5 onto their promoters (FIG. 11B), we identified that knockdown or inhibition of NFAT5 led to a decrease in the protein levels of SNAI1 and ZEB2 (FIG. 9U). This indicates that NFAT5 regulates their post-translational modification or degradation. Furthermore, S100A4 protein expression was also downregulated after depleting Nfat5 (FIG. 9U). Consistently, SNAI1 and ZEB2 among EMT TFs were down-regulated after the knockdown of S100a4 (FIG. 9U). These findings indicate that NFAT5 serves as a master regulator of EMT, while S100A4 functions as a downstream effector. Additionally, TGFβ upregulates NFAT5 (FIG. 9U), and SMADs bind to the NFAT5 promoter (FIG. 11C), providing further support for NFAT5 as a downstream target of canonical TGFβ signaling.

### S1004 Mediates Therapy Resistance Driven by the NFAT5-SMADs Complex

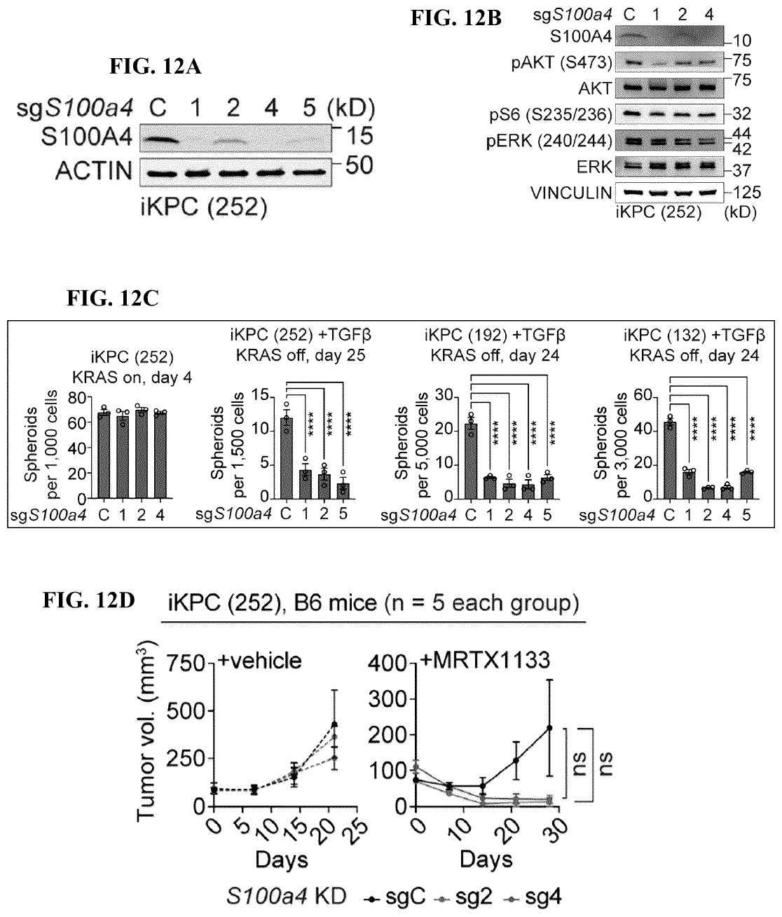

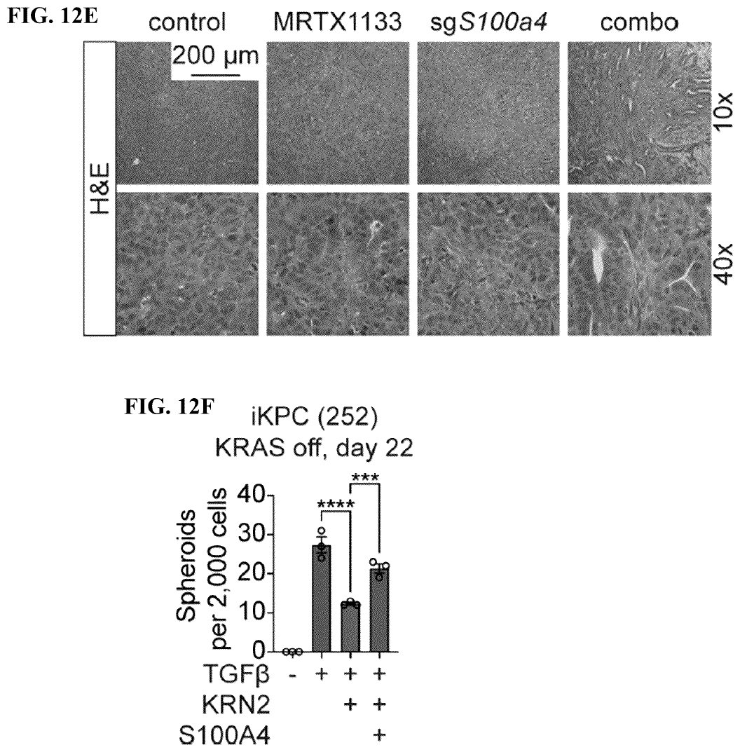

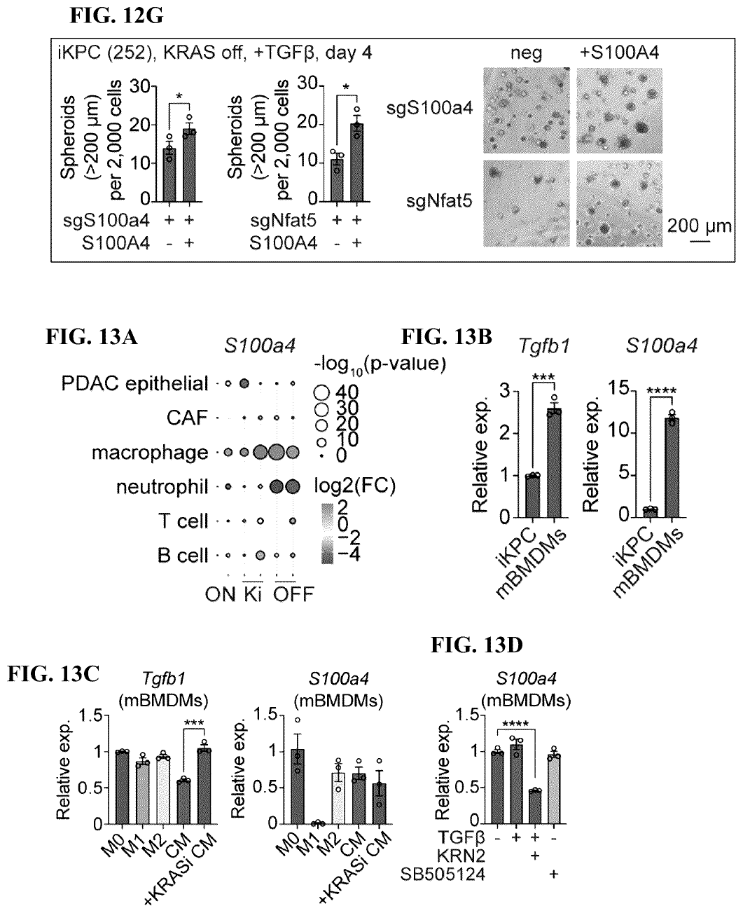

To assess the essential role of S100A4 in TGFβ-driven KRAS* bypass, conducted S100a4 knockdown was analyzed in iKPC cells (FIG. 12A). The suppression of the MAPK and AKT pathways after S100a4 depletion was observed (FIG. 12B). These pathways are the main downstream signaling of KRAS and critical for cancer cell proliferation and survival (Hou and Wang, 2022; Punekar et al., 2022), indicating a potential mechanism by the TGFβ-NFAT5-S100A4 axis to induce KRAS* independency. However, the modest changes in these pathways indicate that other regulators of the MAPK and AKT pathways are present, as well as additional effectors of S100A4 in PDAC that may contribute to therapy resistance.

The results of functional assays demonstrated significant impairment in TGFβ-driven, KRAS*-independent cancer spheroid formation after S100a4 knockdown from three independent iKPC cell lines, while no suppression was observed in KRAS*-expressing cancer spheroids (FIG. 12C). Furthermore, in vivo studies indicated that the loss of S100a4 significantly attenuated tumor growth and led to tumor differentiation in combination with G12Di treatment, particularly when compared with the vehicle control group (FIG. 12D-12E).

S100A4 is a chaperone protein that forms homodimers or heterodimers with its target proteins. These complexes either bind to specific cell surface receptors, activating signaling pathways in the ECM, or modulate the functions of their binding partners in the cytoplasm (Boye and Maelandsmo, 2010). We observed that the addition of recombinant S100A4 protein to the culture media rescued TGFβ-induced, KRAS*-independent cancer spheroid growth following NFAT5 inhibition and after the knockdown of Nfat5 or S100a4 (FIG. 12F-12G). The data indicate that signaling through extracellular S100A4 is required for TGFβ-NFAT5-driven KRAS* bypass, although intracellular S100A4 may also play a role in the process. Taken together, these findings strongly support the notion that S100A4 acts as a key downstream effector in the TGFβ-SMAD3/4-NFAT5 cascade.

### Example 4: Tgfb Stimulates Cancer Cells to Recruit S100A4-Positive Macrophages

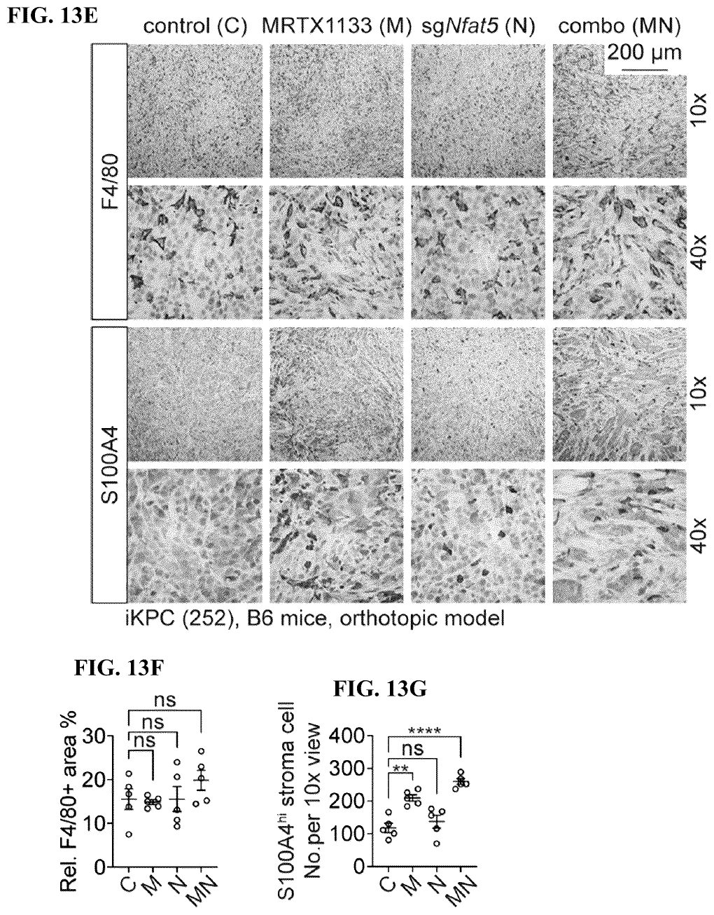

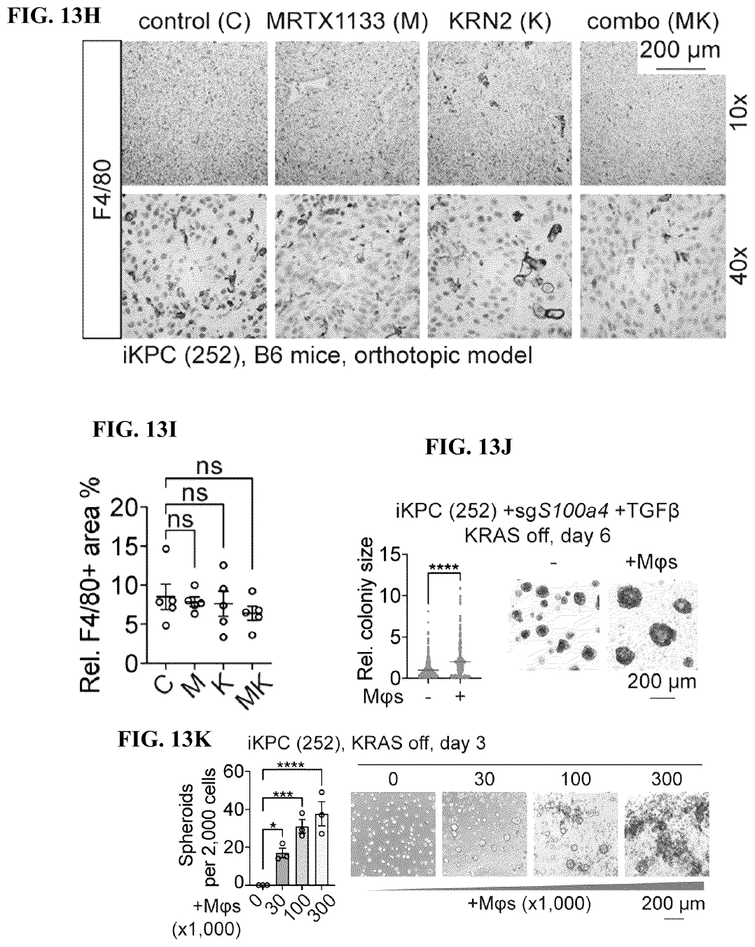

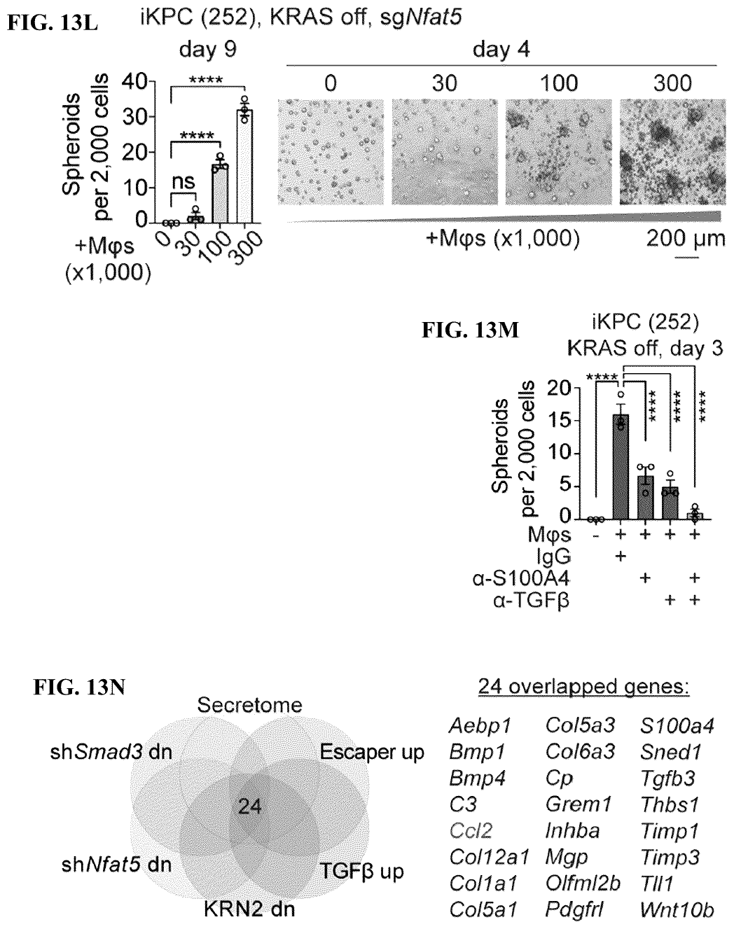

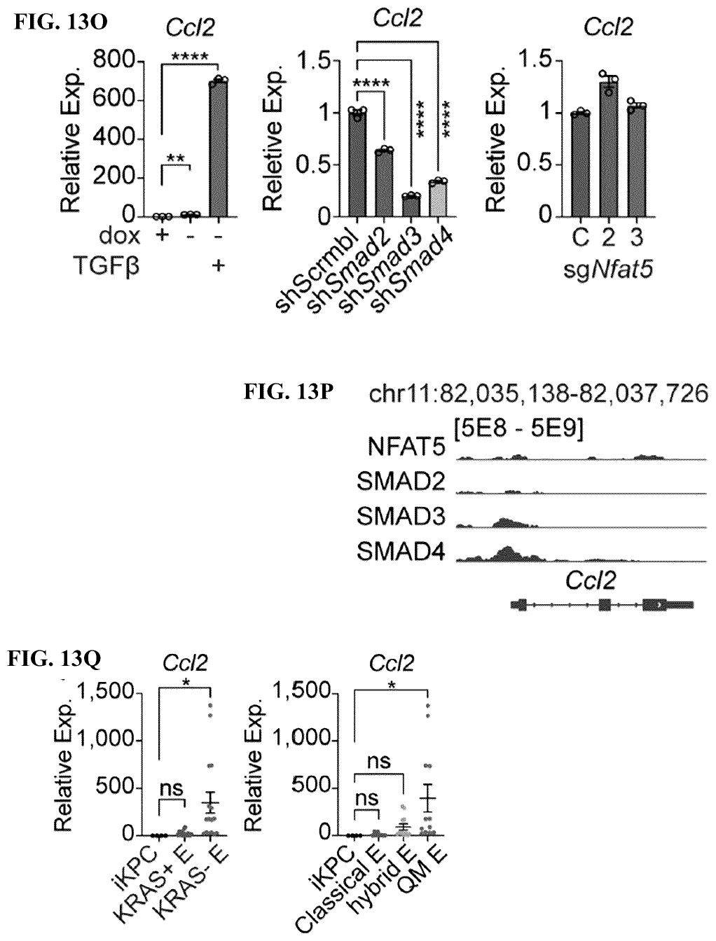

S100A4 is expressed in various cell types, including fibroblasts, immune cells, and endothelial cells³⁵. We observed robust S100A4 expression in stromal cells of both primary and escaper PDAC tumors (FIG. 9I), prompting a comprehensive examination of S100A4 expression in the TME. Utilizing single-cell RNA-sequencing (scRNA-seq) analysis on mouse autochthonous PDAC tumors from both KPC and iKPC models, we found that TAMs expressed significantly higher levels of S100a4 compared to other cell types, irrespective of KRAS* ablation or inhibition (FIG. 13A). This observation was consistent with elevated S100a4 expression in bone marrow-derived macrophages (mBMDMs) from wildtype C57BL/6 mice compared to iKPC PDAC cells (FIG. 13B).

Further analysis revealed predominant expression of S100a4 in M0 and M2 polarized macrophages, with minimal expression in the M1 subtype (FIG. 13C). Conditioned media derived from both KRAS*-expressing and -depleted PDAC cells exhibited a similar effect on inducing S100a4 in macrophages (FIG. 13C). Though TGFβ is also highly expressed in mBMDMs and tumor-educated macrophages (FIG. 13B-13C), the inhibition of NFAT5 suppressed S100a4 expression, while modulation of the TGFβ pathway had no significant impact (FIG. 13D), highlighting the NFAT5-dependent, TGFβ pathway-independent regulation of S100a4 in macrophages.

In Nfat5-knocked down tumors, we observed a reduction in S100A4 expression in tumor cells; however, abundant macrophages and S100A4-positive cells were still present adjacent to tumor cells (FIG. 13E-13G). Additionally, dual inhibition of KRAS* and NFAT5 did not prevent macrophage infiltration (FIG. 13H-13I). We hypothesized that TAMs might promote KRAS* bypass via paracrine S100A4. To verify this, we co-cultured mBMDMs with TGFβ-treated, S100a4-deficient iKPC cancer spheroids following KRAS* ablation. As predicted, the co-culture rescued S100a4 knockdown (FIG. 13J). Moreover, mBMDMs were sufficient to promote KRAS*-independent cancer spheroid growth without the supplement of TGFβ and rescue NFAT5 knockdown (FIG. 13K-13L). Considering that TAMs are a key source of TGFβ to support KRASi resistance¹⁰, we countered S100A4 and TGFβ by neutralizing antibodies in the co-culture system of iKPC PDAC cells and mBMDMs. The observation revealed that the blockade of S100A4 or TGFβ significantly reduced the KRAS*-independent cancer spheroid formation (FIG. 13M). Strikingly, the combination of S100A4 and TGFβ antibodies showed an additive effect that almost completely prevented the KRAS* bypass driven by macrophages (FIG. 13M). We conclude that S100A4 also has intracellular functions that cannot be blocked by S100A4 neutralization but can be compromised by the blockade of the paracrine TGFβ signaling, which inhibits cancer cell-intrinsic elevation of S100a4. Additionally, while the data indicates that environmental S100A4 from non-cancer cells can contribute to KRAS therapy resistance, S100A4 from cancer cells plays the dominant role, as evidenced by the strong tumor ablation following S100a4 knockdown (FIG. 12C-12D).

To elucidate the mechanism underlying the recruitment of macrophages by TGFβ-induced, KRAS*-independent PDAC cells, we analyzed the secretome database from ProteinAtlas and intersected it with our five RNA-seq datasets examining genes upregulated by TGFβ, SMAD3, and NFAT5, as well as genes elevated in escapers (FIG. 13N). Ccl2 emerged as one of the 24 overlapped gene candidates, a well-established chemokine known to recruit CCR2-positive macrophages¹⁰. While KRAS* depletion upregulated Ccl2 expression in iKPC PDAC cells by about 12-fold, TGFβ treatment dramatically elevated Ccl2 to more than 700-fold (FIG. 13O). The upregulation of Ccl2 by TGFβ depended on SMAD3 and SMAD4, not SMAD2 or NFAT5 (FIG. 13O). Correspondingly, we observed binding peaks of SMAD3 and SMAD4 in the promoter region of Ccl2 (FIG. 13P). The expression of Ccl2 was significantly upregulated in KRAS*-independent escaper tumors, especially in the QM subtype, compared with KRAS*-expressing and -reactivated escaper tumors (FIG. 13Q).

### DISCUSSION

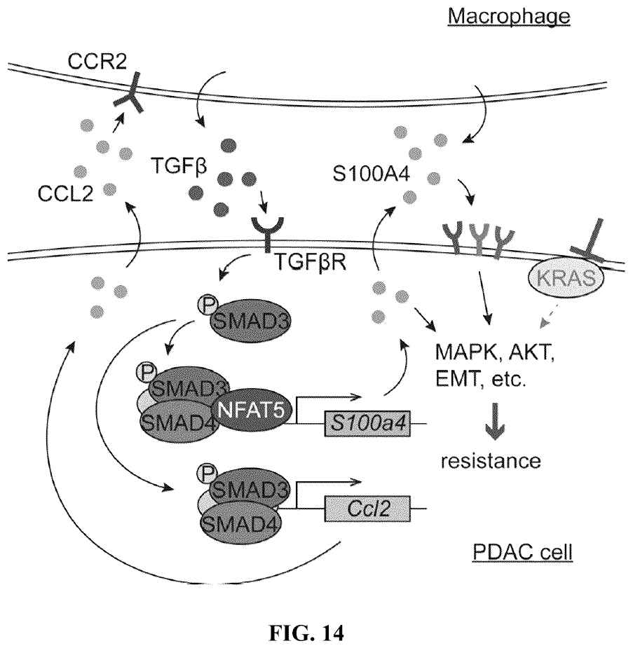

Cancer cells exhibit heterogeneity and hyperplasticity, with EMT serving as a pivotal driver of metastasis and a common adaptive resistance mechanism to various cancer therapies. In this study, we unveil the molecular and cellular mechanisms through which TGFβ, a master regulator of EMT abundant in the TME, fosters resistance to KRAS* targeted therapy (FIG. 14). Our findings elucidate that the nuclear factor NFAT5 interacts with canonical TGFβ pathway key players—SMAD3 and SMAD4—to transcriptionally activate S100a4, a crucial factor for the development of KRAS independence. While conventional Nfat5 knockout mice exhibit high perinatal lethality due to impaired renal and heart functions, the knockout of Nfat5 in adult mice has minimal impact on viability and fertility⁴². Importantly, we demonstrate that NFAT5 is chemically druggable, and mice tolerate the therapy well. Inhibiting NFAT5 not only prevents KRASi resistance but also hampers the growth of QM-like escaper tumors in pre-clinical models. These findings establish a molecular biological foundation for cellular plasticity-associated therapy resistance and propose a strategy to impede this process.

The well-established association between chronic pancreatitis and PDAC is characterized by progressive inflammation and fibrosis (43). Macrophages and TGFβ play pivotal roles in exacerbating this disease (44). Our study reveals that repetitive induction of pancreas injury, mimicking chronic pancreatitis, accelerates resistance to KRAS* targeted therapy in PDAC through the activation of TGFβ pathway. TGFβ from the TME induces Ccl2 expression in cancer cells, initiating a positive feedback loop that further recruits TGFβ-positive macrophages, contributing to cancer cell non-autonomous resistance mechanisms. The upregulation of Ccl2 by TGFβ is dependent on SMAD3 and SMAD4 but independent of NFAT5. Consequently, NFAT5 inhibition alone cannot disrupt macrophage infiltration. Combining an NFAT5 inhibitor with therapies that either block macrophage infiltration (e.g., CCR2 inhibitor) or re-polarize TAMs to stimulate anti-tumor immunity (e.g., CSF1R antibody) may offer a synergistic approach.

The canonical TGFβ pathway exhibits a paradoxical role in cancer, restraining early tumorigenesis while facilitating disease progression and metastasis. SMAD4 is recognized as a prevalent tumor suppressor in PDAC, with its inactivation noted in approximately 30% of patients, correlating with poorer overall survival rates. EMT is evident in SMAD4-deficient patient samples, suggesting a dual regulation of EMT by both canonical and non-canonical TGFβ pathways. Our study underscores the significance of NFAT5 in SMAD4-dependent EMT and KRASi resistance. However, the causal relationship between EMT TFs and KRASi resistance warrants further investigation using loss-of-function methods in genetically engineered mouse PDAC models. The upregulation of EMT TFs through SMAD4-independent mechanisms might override NFAT5 inhibition, thus fostering tumor relapse.

We observed that NFAT5 interacts with SMAD4 only in the nuclei, indicating that DNA is involved in this interaction. It has been reported that NFAT5 can form a homodimer to clamp DNA and stabilize the interaction (Stroud et al., 2002). Due to the low DNA-binding affinity of SMAD3 and SMAD4 (Hill, 2016), nuclear NFAT5 might serve as a crucial pioneer factor for the DNA binding of SMAD3 and SMAD4. Under hypertonic conditions, NFAT5 can be phosphorylated by kinases such as p38 μMAPK, ERK, and CDK5, leading to its activation and translocation into the nucleus (Tong et al., 2006; Zhao et al., 2021). Additionally, methylation of NFAT5 at K668 by the EGFR-EZH2 axis in glioblastoma multiforme has recently been shown to be important for NFAT5 stability, activation, and nuclear accumulation (Li et al., 2023). Thus, the canonical TGFβ pathway and other signaling cascades, including KRAS signaling pathway and the non-canonical TGFβ pathway, cooperatively regulate the downstream effectors of the NFAT5-SMADs complex via posttranslational modification.

Our mechanistic investigations uncover S100A4 as a pivotal downstream effector of the NFAT5-SMADs complex, orchestrating TGFβ-induced resistance to KRAS* targeted therapy. As a chaperone protein devoid of catalytic activity, S100A4 operates through interactions with binding partners. Intracellular S100A4 engages with proteins involved in cell migration, such as actin, while extracellular multimeric forms can bind to receptors like RAGE. The identification of factors interacting with S100A4 to mediate KRAS* targeted therapy resistance necessitates further exploration. Notably, we observe elevated expression of S100A4 in both cancer cells and TAMs. The ability of TAMs to drive KRAS* bypass and rescue the NFAT5-S100A4 axis knockdown underscores their significance as a major contributor to therapy resistance, providing paracrine TGFβ and S100A4. Collectively, our findings offer insights into the molecular underpinnings of cellular plasticity-associated therapy resistance and present novel strategies to target this seemingly “undruggable” process. Moreover, our pre-clinical studies suggest that chronic pancreatitis may pose a potential risk factor for KRAS* targeted therapy resistance, warranting evaluation in ongoing clinical trials.

### REFERENCES FOR EXAMPLES 1-4

1. Punekar, S. R., Velcheti, V., Neel, B. G. & Wong, K. K. The current state of the art and future trends in RAS-targeted cancer therapies. Nat Rev Clin Oncol 19, 637-655 (2022). https://doi.org:10.1038/s41571-022-00671-9
2 Hou, P. & Wang, Y A. Conquering oncogenic KRAS and its bypass mechanisms. Theranostics 12, 5691-5709 (2022). https://doi.org:10.7150/thno.71260
3 Collisson, E. A. et al. Subtypes of pancreatic ductal adenocarcinoma and their differing responses to therapy. Nat Med 17, 500-503 (2011). https://doi.org:10.1038/nm.2344
4 Slater, S. D., Williamson, R. C. & Foster, C. S. Expression of transforming growth factor-beta 1 in chronic pancreatitis. Digestion 56, 237-241 (1995). https://doi.org:10.1159/000201249
5 Riesle, E. et al. Increased expression of transforming growth factor beta s after acute oedematous pancreatitis in rats suggests a role in pancreatic repair. Gut 40, 73-79 (1997). https://doi.org:10.1136/gut.40.1.73
6 Cancer Genome Atlas Research Network. Electronic address, a. a. d. h. e. & Cancer Genome Atlas Research, N. Integrated Genomic Characterization of Pancreatic Ductal Adenocarcinoma. Cancer Cell 32, 185-203 e113 (2017). https://doi.org:10.1016/j.ccell.2017.07.007
7 Ying, H. et al. Oncogenic Kras maintains pancreatic tumors through regulation of anabolic glucose metabolism. Cell 149, 656-670 (2012). https://doi.org:10.1016/j.cell.2012.01.058
8 Wang, X. et al. Identification of MRTX1133, a Noncovalent, Potent, and Selective KRAS(G12D) Inhibitor. J Med Chem 65, 3123-3133 (2022). https://doi.org:10.1021/acs.jmedchem.1c01688
9 Hallin, J. et al. Anti-tumor efficacy of a potent and selective non-covalent KRASG12D inhibitor. Nature Medicine 28, 2171-2182 (2022). https://doi. org:10.1038/s41591-022-02007-7
10 Hou, P. et al. Tumor Microenvironment Remodeling Enables Bypass of Oncogenic KRAS Dependency in Pancreatic Cancer. Cancer Discov 10, 1058-1077 (2020). https://doi.org:10.1158/2159-8290.CD-19-0597
11 Shao, D. D. et al. KRAS and YAP1 converge to regulate EMT and tumor survival. Cell 158, 171-184 (2014). https://doi.org:10.1016/j.cell.2014.06.004
12 Adachi, Y et al. Epithelial-to-Mesenchymal Transition is a Cause of Both Intrinsic and Acquired Resistance to KRAS G12C Inhibitor in KRAS G12C-Mutant Non-Small Cell Lung Cancer. Clin Cancer Res 26, 5962-5973 (2020). https://doi.org:10.1158/1078-0432.CCR-20-2077
13 Peng, D. H. et al. ZEB1 suppression sensitizes KRAS mutant cancers to MEK inhibition by an IL17RD-dependent mechanism. Sci Transl Med 11 (2019). https://doi.org:10.1126/scitranslmed.aag1238
14 Xu, J., Lamouille, S. & Derynck, R. TGF-beta-induced epithelial to mesenchymal transition. Cell Res 19, 156-172 (2009). https://doi.org:10.1038/cr.2009.5
15 Hill, C. S. Transcriptional Control by the SMADs. Cold Spring Harb Perspect Biol 8 (2016). https://doi.org:10.1101/cshperspect.a022079
16 Lee, N., Kim, D. & Kim, W. U. Role of NFAT5 in the Immune System and Pathogenesis of Autoimmune Diseases. Front Immunol 10, 270 (2019). https://doi.org:10.3389/fimmu.2019.00270
17 Ferreira, R. M. M. et al. Duct- and Acinar-Derived Pancreatic Ductal Adenocarcinomas Show Distinct Tumor Progression and Marker Expression. Cell Rep 21, 966-978 (2017). https://doi.org:10.1016/j.celrep.2017.09.093
18 Komar, H. M. et al. Inhibition of Jak/STAT signaling reduces the activation of pancreatic stellate cells in vitro and limits caerulein-induced chronic pancreatitis in vivo. Sci Rep 7, 1787 (2017). https://doi.org:10.1038/s41598-017-01973-0
19 Bardeesy, N. et al. Smad4 is dispensable for normal pancreas development yet critical in progression and tumor biology of pancreas cancer. Genes Dev 20, 3130-3146 (2006). https://doi.org:10.1101/gad.1478706
20 Massague, J. TGFbeta signalling in context. Nat Rev Mol Cell Biol 13, 616-630 (2012). https://doi.org:10.1038/nrm3434
21 Fabregat, I. & Caballero-Diaz, D. Transforming Growth Factor-beta-Induced Cell Plasticity in Liver Fibrosis and Hepatocarcinogenesis. Front Oncol 8, 357 (2018). https://doi.org:10.3389/fonc.2018.00357
22 Zhang, Y E. Non-Smad pathways in TGF-beta signaling. Cell Res 19, 128-139 (2009). https://doi.org: 10. 1038/cr.2008.328
23 Shibue, T. & Weinberg, R. A. EMT, CSCs, and drug resistance: the mechanistic link and clinical implications. Nat Rev Clin Oncol 14, 611-629 (2017). https://doi.org:10.1038/nrclinonc.2017.44
24 Byers, L. A. et al. An epithelial-mesenchymal transition gene signature predicts resistance to EGFR and PI3K inhibitors and identifies Axl as a therapeutic target for overcoming EGFR inhibitor resistance. Clin Cancer Res 19, 279-290 (2013). https://doi.org:10.1158/1078-0432.CCR-12-1558
25 Zheng, X. et al. Epithelial-to-mesenchymal transition is dispensable for metastasis but induces chemoresistance in pancreatic cancer. Nature 527, 525-530 (2015). https://doi.org:10.1038/naturel6064
26 Thillainadesan, G. et al. TGF-beta-dependent active demethylation and expression of the p15ink4b tumor suppressor are impaired by the ZNF217/CoREST complex. Mol Cell 46, 636-649 (2012). https://doi.org:10.1016/j.molcel.2012.03.027
27 Seoane, J. et al. TGFbeta influences Myc, Miz-1 and Smad to control the CDK inhibitor p15INK4b. Nat Cell Biol 3, 400-408 (2001). https://doi.org:10.1038/35070086
28 Kapoor, A. et al. Yap1 activation enables bypass of oncogenic Kras addiction in pancreatic cancer. Cell 158, 185-197 (2014). https://doi.org:10.1016/j.cell.2014.06.003
29 Huang, C. Y et al. Recent progress in TGF-beta inhibitors for cancer therapy. Biomed Pharmacother 134, 111046 (2021). https://doi.org:10.1016/j.biopha.2020.111046
30 Lopez-Rodriguez, C., Aramburu, J., Rakeman, A. S. & Rao, A. NFAT5, a constitutively nuclear NFAT protein that does not cooperate with Fos and Jun. Proc Natl Acad Sci USA 96, 7214-7219 (1999). https://doi.org:10.1073/pnas.96.13.7214
31 Stroud, J. C., Lopez-Rodriguez, C., Rao, A. & Chen, L. Structure of a TonEBP-DNA complex reveals DNA encircled by a transcription factor. Nat Struct Biol 9, 90-94 (2002). https://doi.org:10.1038/nsb749
32 Lopez-Rodriguez, C. et al. Bridging the NFAT and NF-kappaB families: NFAT5 dimerization regulates cytokine gene transcription in response to osmotic stress. Immunity 15, 47-58 (2001). https://doi.org:10.1016/s1074-7613(01)00165-0
33 Miyakawa, H., Woo, S. K., Dahl, S. C., Handler, J. S. & Kwon, H. M. Tonicity-responsive enhancer binding protein, a rel-like protein that stimulates transcription in response to hypertonicity. Proc Natl Acad Sci USA 96, 2538-2542 (1999). https://doi.org:10.1073/pnas.96.5.2538
34 Han, E. J. et al. Suppression of NFAT5-mediated Inflammation and Chronic Arthritis by Novel kappaB-binding Inhibitors. EBioMedicine 18, 261-273 (2017). https://doi.org:10.1016/j.ebiom.2017.03.039
35 Boye, K. & Maelandsmo, G. M. S100A4 and metastasis: a small actor playing many roles. Am J Pathol 176, 528-535 (2010). https://doi.org:10.2353/ajpath.2010.090526
36 Leporcq, C. et al. TFmotifView: a webserver for the visualization of transcription factor motifs in genomic regions. Nucleic Acids Res 48, W208-W217 (2020). https://doi.org:10.1093/nar/gkaa252
37 Che, P. et al. S100A4 promotes pancreatic cancer progression through a dual signaling pathway mediated by Src and focal adhesion kinase. Sci Rep 5, 8453 (2015). https://doi.org:10.1038/srep08453
38 Ai, K. X., Lu, L. Y, Huang, X. Y, Chen, W. & Zhang, H. Z. Prognostic significance of S100A4 and vascular endothelial growth factor expression in pancreatic cancer. World J Gastroenterol 14, 1931-1935 (2008). https://doi.org:10.3748/wjg.14.1931
39 Chen, M., Sinha, M., Luxon, B. A., Bresnick, A. R. & O'Connor, K. L. Integrin alpha6beta4 controls the expression of genes associated with cell motility, invasion, and metastasis, including S100A4/metastasin. The Journal of biological chemistry 284, 1484-1494 (2009). https://doi.org:10.1074/jbc.M803997200
40 Chen, M., Sastry, S. K. & O'Connor, K. L. Src kinase pathway is involved in NFAT5-mediated S100A4 induction by hyperosmotic stress in colon cancer cells. Am J Physiol Cell Physiol 300, C1155-1163 (2011). https://doi.org:10.1152/ajpcell.00407.2010
41 Nieto, M. A., Huang, R. Y, Jackson, R. A. & Thiery, J. P. Emt: 2016. Cell 166, 21-45 (2016). https://doi.org:10.1016/j.cell.2016.06.028
42 Kuper, C., Beck, F. X. & Neuhofer, W. Generation of a conditional knockout allele for the NFAT5 gene in mice. Front Physiol 5, 507 (2014). https://doi.org:10.3389/fphys.2014.00507
43 Zheng, L., Xue, J., Jaffee, E. M. & Habtezion, A. Role of immune cells and immune-based therapies in pancreatitis and pancreatic ductal adenocarcinoma. Gastroenterology 144, 1230-1240 (2013). https://doi.org:10.1053/j.gastro.2012.12.042
44 Xue, J. et al. Alternatively activated macrophages promote pancreatic fibrosis in chronic pancreatitis. Nat Commun 6, 7158 (2015). https://doi.org:10.1038/ncomms8158

### Example 5: Clinical Example

The information herein above can be applied clinically to patients for therapeutic intervention. A preferred embodiment of the invention comprises clinical application of the information described herein to a patient. This can occur after a patient arrives in the clinic and presents with cancer symptoms or symptoms of a tumor. A non-limiting example of an effective dose range for a therapeutic compound described herein is from about 0.1 and 5,000 mg/kg of body weight/per day. One of ordinary skill in the art would be able to study the relevant factors and make the determination regarding the effective amount of the therapeutic compound without undue experimentation.

The therapeutic compounds described herein have been shown to be well tolerated and the symptoms were assessed using clinical scores criteria. The treatment protocol can also optionally include administration of effective amounts of one or more of therapeutic agents that treat or inhibit cancer growth. The treatment protocol can also optionally include administration of effective amounts of one or more of therapeutic agents that treat or inhibit KRASi resistance in tumor cells. Such agents, include without limitation NFAT5 inhibitors or S100a4 inhibitors.

The treatment protocol can also optionally include additional therapeutic compounds, such as KRASi, CCR2i, and/or CSF1R antibodies that help with the treatment of cancer.

### Embodiments

1. A method of treating cancer in a patient, the method comprising administering an effective amount of a KRAS inhibitor and administering an effective amount a TGFβ inhibitor.
2. The method of embodiment 1, wherein the KRAS inhibitor is KRASG12D-LODER, Anti-KRAS G12D mTCR PBL(NCI), MRTX-1133, ASP 3082, BI-1701963, HRS-4642, RMC-9805, UA022, DCTY-1102, or DN-022150.
3. A method of treating cancer in a patient receiving treatment with a KRASi, or having been previously treated with a KRASi, the method comprising administering an effective amount of a TGFβ inhibitor.
4. The method of any one of embodiment 1-3, wherein the TGFβ inhibitor is A77-01, A83-01, AX 12799734, D4476, Distertide, Galunisertib, GW 788388, IN 1130, LY 2109761, R268712, RepSox, SB431542, SB505124, SB525334, SD208 SM16, or a TGFβ antibody.
5. The method of any one of the preceding embodiments, wherein the TGFβ inhibitor is an inhibitor of the canonical TGFβ pathway.
6. The method of any one of the preceding embodiments, wherein the TGFβ inhibitor is a SMAD inhibitor, an NFAT5 inhibitor, a S100A4 inhibitor, or an inhibitor of a downstream EMT transcription factor of SMAD.
7. The method of embodiment 6, wherein the SMAD inhibitor is pirfenidone, SIS3, Halofuginone, asiaticoside, kartogenin, halofuginonoe hydrochloride, trabedersen sodium, nisevokitug, SRI-011381, trimethylamine N-oxide, oxymatrine, Alantolacone, ponsegromab, halofuginone hydrobromide, hydrochlorothiazide, R-268712, luspatercept, disitertide diammonium, 3,3-dimethyl-1-butanol, trimethylamine N-oxide dihydrate, SY-LB-35, Carotuximab, livmoniplimab, trabedersen, (S,R,S)-AHPC-C2-amide-benzofuranylmethyl-pyridine, chebulinic acid, trimethylamine N-oxide-d₉, SJ000063181, CCT365623 hydrochloride, disitertide TFA, isoviolanthin, mongersen, alk5-in-34, elezanumab, IED 2, or Butaprost.
8. The method of embodiment 6 or 7, wherein the NFAT5 inhibitor is KRN2, KRN5, VIVIT, INCA-6, 1IR-VIVIT TFA, PROTAC BTK Degrader-9, KRM-III, NFATc1-IN-1, cyclosporin D, heraclenin, syringaresinol, Q134R, eudebeiolide B, or gomisin E.
9. The method of any one of embodiments 6-8, wherein the S100A4 inhibitor is niclosamide, pentamidine, US-10113, CT070909, or RGC-01-05-18.
10. A method of treating cancer in a patient, the method comprising administering an effective amount of a KRAS inhibitor and administering an effective amount a NFAT5 inhibitor.
11. A method of treating cancer in a patient receiving treatment with a KRASi, or having been previously treated with a KRASi, the method comprising administering an effective amount of a NFAT5 inhibitor.
12. The method of embodiment 10 or embodiment 11, wherein the KRAS inhibitor is KRASG12D-LODER, Anti-KRAS G12D mTCR PBL(NCI), MRTX-1133, ASP 3082, BI-1701963, HRS-4642, RMC-9805, UA022, DCTY-1102, or DN-022150.
13. The method of any one of embodiments 10-12, wherein the NFAT5 inhibitor is KRN2, KRN5, VIVIT, INCA-6, 1 IR-VIVIT TFA, PROTAC BTK Degrader-9, KRM-III, NFATc1-IN-1, cyclosporin D, heraclenin, syringaresinol, Q134R, eudebeiolide B, or gomisin E.
14. The method of anyone of the preceding embodiments, wherein the cancer is therapy resistant and/or an aggressive cancer.
15. The method of any one of the preceding embodiments, wherein, prior to treatment, the cancer is reinitiated after a previous chemotherapy.
16. The method of embodiment 15, herein the previous chemotherapy comprises administration of a KRASi.
17. The method of any one of the preceding embodiments, wherein the cancer is selected from pancreatic ductal adenocarcinoma, acute myeloid leukemia, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastic cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' mor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, glioblastoma, and retinoblastoma.
18. The method of anyone of the preceding embodiments, wherein the cancer is pancreatic ductal adenocarcinoma.
19. The method of anyone of the preceding embodiments further comprising administering an additional therapeutic agent.
20. The method of embodiment 19, wherein the additional therapeutic agent is a compound that acts to block macrophage infiltration and/or acts to re-polarize tumor-associated macrophages to stimulate anti-tumor immunity.
21. The method of embodiment 19 or embodiment 20, wherein the additional therapeutic agent is a CCR2 inhibitor or a CSF1R inhibitor or antibody.
22. The method of embodiment 21, wherein the CCR2 inhibitor is an anti-CCR2 antibody, CCX140, CCX872, PF-04136309 (PF-6309), PF-04178903, INCB-8696, CCX-915, MLN-1202, JNJ-17166864; AZD-2423, INCB-003284, BMS-741672, MK-0812; PF-04634817, CNT0888, or 747 (kaempferol 3-(2,4-di-E-p-coumaroylrhamnoside).
23. The method of embodiment 21, wherein the CSF1R inhibitor or antibody is pexidartinib, emactuzumab, cabiralizumab, ARRY-382, BLZ945, AJUD010, AMG820, IMC-CS4, JNJ-40346527, PLX5622, or FPA008.
24. The method of any one of the preceding embodiments, wherein the KRASi and the TGFβi or NFAT5i act synergistically.
25. The method of any one of the preceding embodiments, further comprising assessing the patient for a reduction in cancer symptoms.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treating cancer in a patient, the method comprising administering an effective amount of a KRAS inhibitor and administering an effective amount a TGFβ inhibitor.

2. The method of claim 1, wherein the KRAS inhibitor is KRASG12D-LODER, Anti-KRAS G12D mTCR PBL(NCI), MRTX-1133, ASP 3082, BI-1701963, HRS-4642, RMC-9805, UA022, DCTY-1102, or DN-022150.

3. A method of treating cancer in a patient receiving treatment with a KRASi, or having been previously treated with a KRASi, the method comprising administering an effective amount of a TGFβ inhibitor.

4. The method of claim 1, wherein the TGFβ inhibitor is A77-01, A83-01, AX 12799734, D4476, Distertide, Galunisertib, GW 788388, IN 1130, LY 2109761, R 268712, RepSox, SB431542, SB505124, SB525334, SD208 SM16, or a TGFβ antibody.

5. The method of claim 1, wherein the TGFβ inhibitor is an inhibitor of the canonical TGFβ pathway.

6. The method of claim 1, wherein the TGFβ inhibitor is a SMAD inhibitor, an NFAT5 inhibitor, a S100A4 inhibitor, or an inhibitor of a downstream EMT transcription factor of SMAD.

7. The method of claim 6, wherein
a. the SMAD inhibitor is pirfenidone, SIS3, Halofuginone, asiaticoside, kartogenin, halofuginonoe hydrochloride, trabedersen sodium, nisevokitug, SRI-011381, trimethylamine N-oxide, oxymatrine, Alantolacone, ponsegromab, halofuginone hydrobromide, hydrochlorothiazide, R-268712, luspatercept, disitertide diammonium, 3,3-dimethyl-1-butanol, trimethylamine N-oxide dihydrate, SY-LB-35, Carotuximab, livmoniplimab, trabedersen, (S,R,S)-AHPC-C2-amide-benzofuranylmethyl-pyridine, chebulinic acid, trimethylamine N-oxide-d₉, SJ000063181, CCT365623 hydrochloride, disitertide TFA, isoviolanthin, mongersen, alk5-in-34, elezanumab, IED 2, or Butaprost;
b. the NFAT5 inhibitor is KRN2, KRN5, VIVIT, INCA-6, 11R-VIVIT TFA, PROTAC BTK Degrader-9, KRM-III, NFATc1-IN-1, cyclosporin D, heraclenin, syringaresinol, Q134R, eudebeiolide B, or gomisin E; or
c. the S100A4 inhibitor is niclosamide, pentamidine, US-10113, CT070909, or RGC-01-05-18.

8. A method of treating cancer in a patient receiving treatment with a KRASi, or having been previously treated with a KRASi, the method comprising administering an effective amount of a NFAT5 inhibitor.

9. The method of claim 1, wherein the cancer is therapy resistant and/or an aggressive cancer.

10. The method of claim 1, wherein, prior to treatment, the cancer is reinitiated after a previous chemotherapy.

11. The method of claim 10, herein the previous chemotherapy comprises administration of a KRASi.

12. The method of claim 1, wherein the cancer is selected from pancreatic ductal adenocarcinoma, acute myeloid leukemia, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastic cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' mor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, glioblastoma, and retinoblastoma.

13. The method of claim 1, wherein the cancer is pancreatic ductal adenocarcinoma.

14. The method of claim 1 further comprising administering an additional therapeutic agent.

15. The method of claim 14, wherein the additional therapeutic agent is a compound that acts to block macrophage infiltration and/or acts to re-polarize tumor-associated macrophages to stimulate anti-tumor immunity.

16. The method of claim 14, wherein the additional therapeutic agent is a CCR2 inhibitor or a CSF1R inhibitor or antibody.

17. The method of claim 16, wherein
a. the CCR2 inhibitor is an anti-CCR2 antibody, CCX140, CCX872, PF-04136309 (PF-6309), PF-04178903, INCB-8696, CCX-915, MLN-1202, JNJ-17166864; AZD-2423, INCB-003284, BMS-741672, MK-0812; PF-04634817, CNT0888, or 747 (kaempferol 3-(2,4-di-E-p-coumaroylrhamnoside); or
b. the CSF1R inhibitor or antibody is pexidartinib, emactuzumab, cabiralizumab, ARRY-382, BLZ945, AJUD010, AMG820, IMC-CS4, JNJ-40346527, PLX5622, orFPA008.

18. The method of claim 1, wherein the KRASi and the TGFβi or NFAT5i act synergistically.

19. The method of claim 1, further comprising assessing the patient for a reduction in cancer symptoms.
